(19) United States
(12) Patent Application Publication
(10) Pub. No.: US 2026/0317298 A1
(43) Pub. Date: Oct. 15, 2026

(54) Cereblon Ligands and Uses Thereof

(71) Applicants: REGENTS OF THE UNIVERSITY OF MICHIGAN, Ann Arbor, MI (US); ONCOPIA THERAPEUTICS, INC. d.b.a SK LIFE SCIENCE LABS, King of Prussia, PA (US)

(72) Inventors: Guozhang Xu, New York, NY (US); Zhenwu Li, New York, NY (US); Shaomeng Wang, Superior Township, MI (US); Xuqing Zhang, New York, NY (US)

(21) Appl. No.: 18/881,943

(22) PCT Filed: Jul. 11, 2023
(86) PCT No.: PCT/US23/27353

§ 371 Date: Jan. 7, 2025

Related U.S. Application Data
(60) Provisional application No. 63/388,297, filed on Jul. 12, 2022.
(60) Provisional application No. 63/388,299, filed on Jul. 12, 2022.
(60) Provisional application No. 63/388,300, filed on Jul. 12, 2022.
(60) Provisional application No. 63/388,302, filed on Jul. 12, 2022.
(60) Provisional application No. 63/408,744, filed on Sep. 21, 2022.
(60) Provisional application No. 63/408,758, filed on Sep. 21, 2022.
(60) Provisional application No. 63/408,601, filed on Sep. 21, 2022.
(60) Provisional application No. 63/408,633, filed on Sep. 21, 2022.
(60) Provisional application No. 63/412,346, filed on Sep. 30, 2022.
(60) Provisional application No. 63/427,277, filed on Nov. 22, 2022.
(60) Provisional application No. 63/460,734, filed on Apr. 20, 2023.

(51) Int. Cl. C07D 401/12; A61K 31/4525; A61K 31/4545; A61K 31/5383; A61K 47/54; A61K 47/55; C07D 405/12; C07D 491/107; C07D 498/04

(52) U.S. Cl. CPC C07D 401/12; A61K 31/4525; A61K 31/4545; A61K 31/5383; A61K 47/545; A61K 47/55; C07D 405/12; C07D 491/107; C07D 498/04

(57) ABSTRACT

Described herein are compounds and conjugates of Formulae I and II and their pharmaceutically acceptable salts, solvates, or stereoisomers thereof, as well as their uses (e.g., as cereblon-binding agents and bifunctional degraders for certain proteins).

### RELATED APPLICATIONS

This application claims the benefit of and priority to U.S. Provisional Application No. 63/412,346, filed Sep. 30, 2022; U.S. Provisional Application No. 63/388,300, filed Jul. 12, 2022; U.S. Provisional Application No. 63/408,744, filed Sep. 21, 2022; U.S. Provisional Application No. 63/427,277, filed Nov. 22, 2022; U.S. Provisional Application No. 63/460,734, filed Apr. 20, 2023; U.S. Provisional Application No. 63/388,302, filed Jul. 12, 2022; U.S. Provisional Application No. 63/408,758, filed Sep. 21, 2022; U.S. Provisional Application No. 63/388,297, filed Jul. 12, 2022; U.S. Provisional Application No. 63/408,601, filed Sep. 21, 2022; U.S. Provisional Application No. 63/388,299, filed Jul. 12, 2022; and U.S. Provisional Application No. 63/408,633, filed Sep. 21, 2022; the contents of each of which are incorporated herein by reference in their entireties.

### BACKGROUND

Cereblon (CRBN), a component of the DDBI-CUL4a-Roclubiquitin ligase complex, is a molecular target of immunomodulatory agents such as thalidomide, lenalidomide, and pomalidomide. Inhibition of CRBN ubiquitination by these agents may allow CRBN to accumulate, leading to the increased cullin-4 RING E3 ligase-mediated degradation of target proteins.

The discovery process of CRBN type E3 ligase ligand is related to the study of thalidomide's mechanism of action. In 2010, while studying the toxicity of thalidomide, scientists discovered that cereblon is a binding protein of thalidomide (Science 2010, 327, 1345). Cerebellar protein is part of the E3 ubiquitin ligase protein complex, which acts as a substrate receptor to select ubiquitinated proteins. The study shows that thalidomide-cerebellar protein binding in vivo may be the cause of thalidomide teratogenicity. Subsequent studies found that the compound and related structures can be used as anti-inflammatory agents, anti-angiogenic agents and anti-cancer agents. Lenalidomide and pomalidomide obtained by further modification of the structure of thalidomide have greatly improved their safety and significantly reduced their teratogenic effects. Lenalidomide has been approved by the FDA in 2006 for marketing. Two groundbreaking papers published in Science in 2014 pointed out that lenalidomide works by degrading two special B cell transcription factors, Ikaros family zinc finger structural proteins 1 and 3 (IKZF1 and IKZF3), which further reveals the structure of thalidomide may be combined with the E3 ubiquitin ligase protein complex of the cerebellar protein to further play a role in degrading the target protein (Science, 2014, 343, 301; Science, 2014, 343, 305).

On this basis, CRBN ligands are widely used in protein degradation, and a series of PROTACs molecules based on CRBN ligands have been developed. Due to the influence of CRBN ligand itself on the target point, it may additionally degrade zinc finger domain protein.

Therefore, the design and synthesis of new and highly selective CRBN ligands is also particularly important in the synthesis of PROTACs molecules.

### SUMMARY

In certain aspects, the present disclosure provides compounds or conjugates of Formula II:

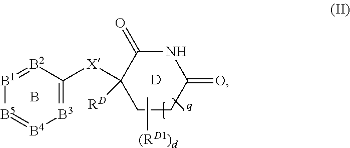

and pharmaceutically acceptable salts, solvates, or stereoisomers thereof, wherein each of the variables in Formula II is described, embodied, and exemplified herein.

In certain aspects, the present disclosure provides compounds or conjugates of Formula I:

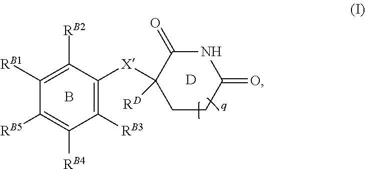

and pharmaceutically acceptable salts, solvates, or stereoisomers thereof, wherein each of the variables in Formula I is described, embodied, and exemplified herein.

In certain aspects, the present disclosure provides pharmaceutical compositions comprising a compound disclosed herein, and a pharmaceutically acceptable excipient.

In certain aspects, provided herein are methods of binding cereblon E3 ubiquitin ligase protein complex in a subject or biological sample comprising administering a compound described herein to the subject or contacting the biological sample with a compound described herein.

In certain aspects, provided herein are uses of a compound described herein in the manufacture of a medicament for binding cerebon E3 ubiquitin ligase protein complex in a subject or biological sample.

In certain aspects, provided herein are compounds described herein for use in binding cereblon E3 ubiquitin ligase protein complex in a subject or biological sample.

In certain aspects, provided herein are methods of degrading a protein in a subject or biological sample comprising administering a compound described herein to the subject or contacting the biological sample with a compound described herein.

In certain aspects, provided herein are uses of a compound described herein in the manufacture of a medicament for degrading a protein in a subject or biological sample.

In certain aspects, provided herein are compounds described herein for use in degrading a protein in a subject or biological sample.

In certain aspects, provided herein are methods of reducing a protein in a subject or biological sample comprising administering a compound described herein to the subject or contacting the biological sample with a compound described herein.

In certain aspects, provided herein are uses of a compound described herein in the manufacture of a medicament for reducing a protein in a subject or biological sample.

In certain aspects, provided herein are compounds described herein for use in reducing a protein in a subject or biological sample.

In certain aspects, provided herein are methods of treating or preventing a disease or disorder a subject in need thereof, comprising administering to the subject a compound described herein.

In certain aspects, provided herein are uses of a compound described herein in the manufacture of a medicament for treating or preventing a disease or disorder in a subject in need thereof.

In certain aspects, provided herein are compounds described herein for use in treating or preventing a disease or disorder in a subject in need thereof.

### DETAILED DESCRIPTION

The present disclosure relates to compounds that potentially show cereblon-binding activity, and pharmaceutical compositions comprising such compounds.

Compounds of the Present Disclosure Cereblon Ligands

In certain aspect, the present disclosure provides compounds of Formula II:

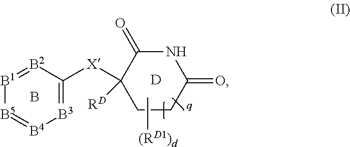

or a pharmaceutically acceptable salt, solvate, or stereoisomer thereof, wherein:
B¹ is N or CR^{B1};
B² is N or CR^{B2};
B³ is N or CR^{B3};
B⁴ is N or CR^{B4};
B⁵ is N or CR^{B5};
wherein:
i) B⁵ is CR^{B5}, and either R^{B1} and R^{B5} or R^{B4} and R^{B5}, together with the carbon atoms to which they are bonded, form Ring A, wherein Ring A is optionally substituted C₅₋₁₆ fused or spiro carbocycle or 5- to 16-membered fused or spiro heterocycle;
the remaining of R^{B1}, R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.
or
ii) R^{B5} is

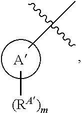

wherein:
Ring A′ is C₄₋₁₂ fused carbocyclyl or 4- to 12-membered fused heterocyclyl;
each R^{A′} is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 10, as valency permits;
the remaining of R^{B1}, R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
X′ is —O— or —NR^{X′}—;
R^{X′} is hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u};
each R^{D}1 is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 6;
R^{D} is hydrogen, deuterium, C₁₋₆ haloalkyl, or C₁₋₆ alkyl; and
q is an integer from 0 to 2,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3 to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, B¹ is N or CR^{B1}. In certain embodiments, B¹ is N. In certain embodiments, B¹ is CR^{B1}.

In certain embodiments, B² is N or CR^{B2}. In certain embodiments, B² is N. In certain embodiments, B² is CR^{B2}.

In certain embodiments, B³ is N or CR^{B3}. In certain embodiments, B³ is N. In certain embodiments, B³ is CR^{B3}.

In certain embodiments, B⁴ is N or CR^{B4}. In certain embodiments, B² is N. In certain embodiments, B⁴ is CR^{B4}.

In certain embodiments, B⁵ is N or CR^{B5}. In certain embodiments, B² is N. In certain embodiments, B⁵ is CR^{B5}.

In certain embodiments, B⁵ is CR^{B5}, either R^{B1} and R^{B5} or R^{B4} and R^{B5}, together with the carbon atoms to which they are bonded, form Ring A, wherein Ring A is optionally substituted fused or spiro C₅₋₁₆ carbocycle or 5- to 16-membered fused or spiro heterocycle.

In certain embodiments, one of R^{B2}, R^{B3}, and R^{B4} or one of R^{B1}, R^{B2}, and R^{B3} is N. In certain embodiments, two of R^{B2}, R^{B3}, and R^{B4} or two R^{B1}, R^{B2}, and R^{B3} are N.

In certain embodiments, the remaining of RB, R^{B2}, R^{B3}, R^{B4}, and R^{B5}, when applicable, are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, 0, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, 0, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{C}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, the remaining of RB, R^{B2}, R^{B3}, R^{B4}, and R^{B5}, when applicable, are independently hydrogen, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, the remaining of R^{B1}, R^{B2}, R^{B3}, R^{B4}, and R^{B5}, when applicable, are independently hydrogen, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, the remaining of R^{B1}, R^{B2}, R^{B3}, R^{B4}, and R^{B5}, when applicable, are independently hydrogen, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, the remaining of R^{B1}, R^{B2}, R^{B3}, R^{B4}, and R^{B5}, when applicable, are independently hydrogen, halogen, —CN, —NO2, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each of the remaining of R^{B1}, R^{B2}, R^{B3}, R^{B4}, and R^{B5}, when applicable, is hydrogen, halogen, or C₁₋₆ alkyl.

In certain embodiments, Ring A is optionally substituted with one or more substituents selected from oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{C}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, Ring A is optionally substituted with one or more R^{u}, R^{A1}, R^{A1′}, R^{A2}, or R^{A2′}.

In certain embodiments, R^{u} is R^{A1}. In certain embodiments, R^{u} is R^{A1′}. In certain embodiments, R^{u} is R^{A2}. In certain embodiments, R^{u} is R^{A2′}.

In certain embodiments, Ring A is optionally substituted with one or more R^{u}, R^{A}, R^{Ax}, R^{A1}, or R^{A2}.

In certain embodiments, R^{u} is R^{A1}. In certain embodiments, R^{u} is R^{Ax}. In certain embodiments, R^{u} is R^{A1}. In certain embodiments, R^{u} is R^{A2}.

In certain embodiments, Ring A is:

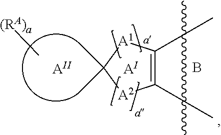

wherein:
Ring A^{II} is C₃₋₈ carbocycle or 3- to 8-membered heterocycle;
each A¹ is independently —C(R^{A1})₂—, —NR^{A1′}—, —O—, —S—, —S(═O)—, or —S(═O)₂—;
each A² is independently —C(R^{A2})₂—, —NR^{A2′}—, —O—, —S—, —S(═O)—, or —S(═O)₂—;
each occurrence of R^{A′} and R^{A2} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{A1} or two geminal R^{A2} together form oxo; or
two geminal R^{A1} or two geminal R^{A2}, together with the carbon atom to which they are attached, form C₃₋₆ carbocycle or 3- to 6-membered heterocycle, wherein the carbocycle or heterocycle is optionally substituted with one or more R^{u};
each occurrence of R^{A1′} and R^{A2′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
a′ and a″ are independently an integer selected from 0 to 2, wherein a′ and a″ are not both 0;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
a is an integer selected from 0 to 8, as valency permits.

In certain embodiments, wherein Ring A^{I} is not carbocycle.

In certain embodiments, Ring A^{I} is 5- to 8-membered heterocycle (e.g., heterocycle comprising one or two 3- to 8-membered rings and 1-5 heteroatoms selected from N, 0, and S).

In certain embodiments, Ring A^{I} is 5- to 8-membered heterocycle comprising one oxygen atom. In certain embodiments, Ring A^{I} is 5- to 8-membered heterocycle comprising one oxygen atom and one double bond. In certain embodiments, Ring A^{I} is 5- to 8-membered heterocycle comprising two oxygen atoms. In certain embodiments, Ring A^{I} is 5- to 8-membered heterocycle comprising one nitrogen atom. In certain embodiments, Ring A^{I} is 5- to 8-membered heterocycle comprising two nitrogen atoms. In certain embodiments, Ring A^{I} is 5- to 8-membered heterocycle comprising one nitrogen atom and one oxygen atom.

In certain embodiments, Ring A^{II} is C₃₋₈ carbocycle (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), or bicyclo[2.2.2]octanyl (C₈)) or 3- to 8-membered heterocyclyl (e.g., heterocyclyl comprising one or two 3- to 8-membered rings and 1-5 heteroatoms selected from N, O, and S).

In certain embodiments, each A¹ is independently —C(R^{A1})₂—, —NR^{A1′}—, —O—, —S—, —S(═O)—, or —S(═O)₂—.

In certain embodiments, each A¹ is independently —C(R^{A1})₂—, —NR^{A1′}—, or —O—.

In certain embodiments, each A² is independently —C(R^{A2})₂—, —NR^{A2′}—, —O—, —S—, —S(═O)—, or —S(═O)₂—.

In certain embodiments, each A² is independently —C(R^{A2})₂—, —NR^{A2′}—, or —O—.

In certain embodiments, each occurrence of R^{A1} and R^{A2} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{A1} and R^{A2} is independently hydrogen, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{A′} and R^{A2} is independently hydrogen, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{A′} and R^{A2} is independently hydrogen, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each occurrence of R^{A′} and R^{A2} is independently hydrogen, halogen, —CN, —NO2, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each occurrence of R^{A′} and R^{A2} is hydrogen.

In certain embodiments, two geminal R^{A1} or two geminal R^{A2} together form oxo.

In certain embodiments, two geminal R^{A1} or two geminal R^{A2}, together with the carbon atom to which they are attached, form C₃₋₆ carbocycle (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, 0, and S), wherein the carbocycle or heterocycle is optionally substituted with one or more R^{u}.

In certain embodiments, each occurrence of R^{A1′} and R^{A2′} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{A1′} and R^{A2′} is independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{A1′} and R^{A2′} is independently hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, each occurrence of R^{A1′} and R^{A2′} is independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, each occurrence of R^{A1′} and R^{A2′} is independently hydrogen or C₁₋₆ alkyl.

In certain embodiments, each occurrence of R^{A1′} and R^{A2′} is hydrogen.

In certain embodiments, a′ is 0. In certain embodiments, a′ is 1. In certain embodiments, a′ is 2.

In certain embodiments, a″ is 0. In certain embodiments, a″ is 1. In certain embodiments, a″ is 2.

In certain embodiments, one of a′ and a″ is 0.

In certain embodiments, each of a′ and a″ is 1.

In certain embodiments, one of a′ and a″ is 1, and the other one of a′ and a″ is 2.

In certain embodiments, each R^{A} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{A} is independently oxo, halogen, —CN, —NO2, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, a is 0. In certain embodiments, a is 1. In certain embodiments, a is 2. In certain embodiments, a is 3. In certain embodiments, a is 4, as valency permits. In certain embodiments, a is 5, as valency permits. In certain embodiments, a is 6, as valency permits. In certain embodiments, a is 7, as valency permits. In certain embodiments, a is 8. In certain embodiments, a is 8, as valency permits.

In certain embodiments, a is 0.

In certain embodiments, each R^{A} may be present on either Ring A^{I} or Ring A^{II}.

In certain embodiments, Ring A is:

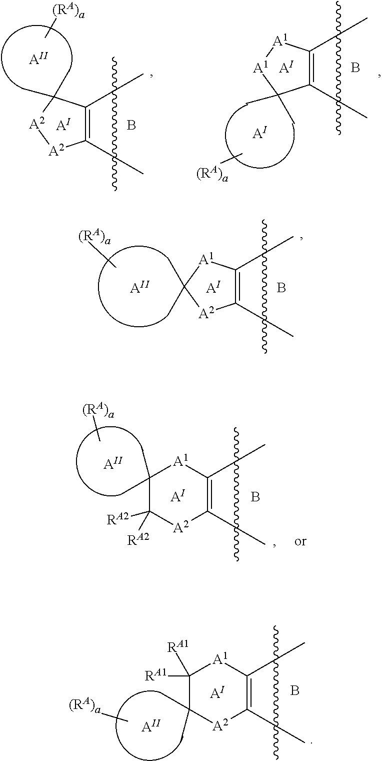

In certain embodiments, the compound of Formula II is a compound of Formula II-1

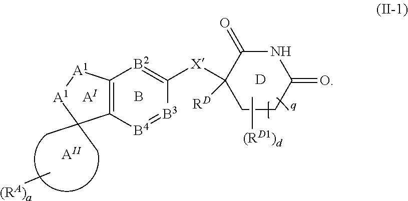

In certain embodiments, Ring A is

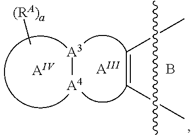

wherein:
Ring A^{III} and Ring A^{IV} are independently C₄₋₈ carbocycle or 4- to 8-membered heterocycle; wherein at least one of Ring A^{III} and Ring A^{IV} is 4- to 8-membered heterocycle;
A³ and A⁴ are independently C, CR^{Ax}, or N;
R^{Ax} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
a is an integer selected from 0 to 8, as valency permits,
wherein R^{A} may be present on either Ring A^{III} or Ring A^{IV}.

In certain embodiments, Ring A^{III} and Ring A^{IV} are independently C₄₋₈ carbocycle (e.g., cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), or cyclooctenyl (C₈)) or 4- to 8-membered heterocycle (e.g., heterocycle comprising one or two 4- to 8-membered rings and 1-3 heteroatoms selected from N, O, and S).

In certain embodiments, Ring A^{IV} is 5- to 8-membered heterocycle comprising at least two nitrogen atoms. In certain embodiments, Ring A^{IV} is 5- to 8-membered heterocycle comprising two nitrogen atoms.

In certain embodiments, A³ and A⁴ are independently C, CR^{Ax}, or N.

In certain embodiments, one of A³ and A⁴ is CR^{Ax}, and the other one of A³ and A⁴ is N.

In certain embodiments, each R^{Ax} is independently hydrogen, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, 0, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, 0, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{Ax} is independently hydrogen, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{Ax} is independently hydrogen, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{Ax} is independently hydrogen, halogen, —CN, —NO2, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R^{Ax} is hydrogen.

In certain embodiments, Ring A is

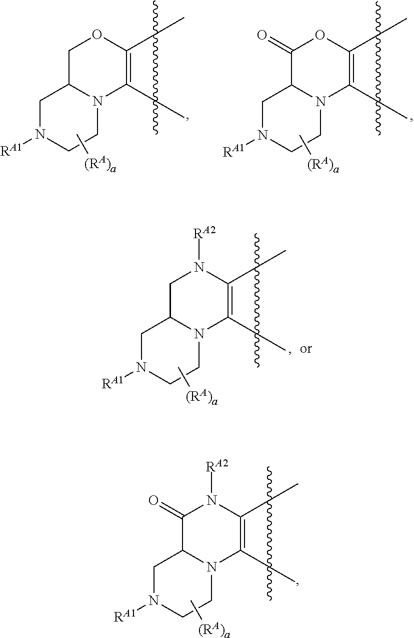

wherein:
R^{A5} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}; or
R^{A5} is an amino-protecting group; and
R^{A6} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}; or
R^{A6} is an amino-protecting group.

In certain embodiments, the compound of Formula II is a compound of Formula II-3

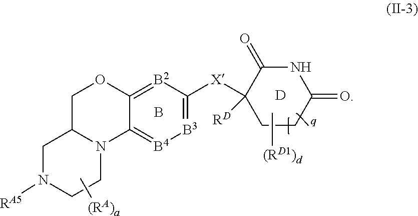

In certain embodiments, R^{A5} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), —(C₁₋₆ alkylene)-(5- to 10-membered heteroaryl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}.

In certain embodiments, R^{A5} is hydrogen, C₁₋₆ alkyl, C₃₋₆ carbocyclyl, 3- to 6-membered heterocyclyl, C₆ aryl, 5- to 6-membered heteroaryl, —(C₁₋₆ alkylene)-(C₃₋₆ carbocyclyl), —(C₁₋₆ alkylene)-(3- to 6-membered heterocyclyl), —(C₁₋₆ alkylene)-(C₆ aryl), —(C₁₋₆ alkylene)-(5- to 6-membered heteroaryl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, carbocyclyl, heterocyclyl, aryl, or heteroaryl is optionally substituted with one or more R^{u}.

In certain embodiments, R^{A5} is hydrogen, C₁₋₆ alkyl, C₃₋₆ carbocyclyl, 3- to 6-membered heterocyclyl, C₆ aryl, 5- to 6-membered heteroaryl, —(C₁₋₆ alkylene)-(C₃₋₆ carbocyclyl), —(C₁₋₆ alkylene)-(3- to 6-membered heterocyclyl), —(C₁₋₆ alkylene)-(C₆ aryl), —(C₁₋₆ alkylene)-(5- to 6-membered heteroaryl), —C(═O)R^{a}, —C(═O)OR^{b}, or —C(═O)NR^{c}R^{d}, wherein the alkyl, alkylene, carbocyclyl, heterocyclyl, aryl, or heteroaryl is optionally substituted with one or more R^{u}.

In certain embodiments, R^{A5} is hydrogen, C₁₋₆ alkyl, —(C₁₋₆ alkylene)-(C₃₋₆ carbocyclyl), —(C₁₋₆ alkylene)-(3- to 6-membered heterocyclyl), —(C₁₋₆ alkylene)-(C₃₋₆ aryl), -—(C₁₋₆ alkylene)-(5- to 6-membered heteroaryl), —C(═O)R^{a}, or —C(═O)OR^{b}, wherein the alkyl, alkylene, carbocyclyl, heterocyclyl, aryl, or heteroaryl is optionally substituted with one or more R^{u}.

In certain embodiments, R^{A5} is an amino-protecting group.

In certain embodiments, R^{A6} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{A6} is hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{A6} is hydrogen, C₁₋₆ alkyl, C₂₋₆ alkenyl, C₂₋₆ alkynyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R^{A6} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, R^{A6} is hydrogen or C₁₋₆ alkyl.

In certain embodiments, R^{A6} is an amino-protecting group.

In certain embodiments, each R^{A} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{A} is independently oxo, halogen, —CN, —NO2, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, a is 0. In certain embodiments, a is 1. In certain embodiments, a is 2. In certain embodiments, a is 3. In certain embodiments, a is 4, as valency permits. In certain embodiments, a is 5, as valency permits. In certain embodiments, a is 6, as valency permits. In certain embodiments, a is 7, as valency permits. In certain embodiments, a is 8, as valency permits.

In certain embodiments, a is 0.

In certain embodiments, R^{B5} is

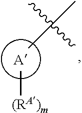

wherein:
Ring A′ is C₄₋₁₂ fused carbocyclyl or 4- to 12-membered fused heterocyclyl;
each R^{A′} is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 10, as valency permits.

In certain embodiments, Ring A′ is C₄₋₁₂ fused carbocyclyl or 4- to 12-membered fused heterocyclyl (e.g., heterocyclyl comprising two 3- to 6-membered fused rings and 1-3 heteroatoms selected from N, O, and S).

In certain embodiments, Ring A′ is 4- to 12-membered fused heterocyclyl.

In certain embodiments, Ring A′ is 5- to 12-membered fused heterocyclyl comprising 1 or 2 nitrogen atoms.

In certain embodiments, each R^{A′} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A′} is independently oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A′} is independently oxo, halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{A′} is independently oxo, halogen, —CN, —NO2, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, at least one R^{A′} is halogen. In certain embodiments, at least one R^{A′} is —F.

In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10.

In certain embodiments, m is an integer selected from 0 to 3.

In certain embodiments, R^{D} is hydrogen, deuterium, or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, R^{D} is hydrogen.

In certain embodiments, q is 0. In certain embodiments, q is 1. In certain embodiments, q is 2.

In certain embodiments, q is 1.

In certain embodiments, each R^{D}1 is independently halogen (e.g., —F, —Cl, —Br, or —I), —CN, —NO2, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D}1 is independently halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D}1 is independently halogen, —CN, —NO2, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{D}1 is independently halogen, —CN, —NO2, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, d is 0. In certain embodiments, d is 1. In certain embodiments, d is 2. In certain embodiments, d is 3. In certain embodiments, d is 4. In certain embodiments, d is 5.

In certain embodiments, d is 0.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), orhexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{a} is independently C₁₋₆ alkyl, C₂₋₆ alkenyl, C₂₋₆ alkynyl, C₃₋₆ carbocyclyl, or 3- to 6-membered heterocyclyl.

In certain embodiments, each R^{a} is independently C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁₋₆ alkyl, C₂₋₆ alkenyl, C₂-6 alkynyl, C₃₋₆ carbocyclyl, 3- to 6-membered heterocyclyl, C₆ aryl, or 5- to 6-membered heteroaryl.

In certain embodiments, each R^{b} is independently hydrogen, C₁₋₆ alkyl, C₂₋₆ alkenyl, C₂-6 alkynyl, C₃₋₆ carbocyclyl, or 3- to 6-membered heterocyclyl.

In certain embodiments, each R^{b} is independently hydrogen, C₁₋₆ alkyl, C₃₋₆ carbocyclyl, or 3- to 6-membered heterocyclyl, or C₂₋₆ alkynyl, wherein the alkyl, carbocyclyl, or heterocyclyl is optionally substituted with one or more R^{u}.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, 0, and S), wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, R^{a}, R^{b}, R^{c}, and R^{d} is independently and optionally substituted with one or more R^{z}.

In certain embodiments, R^{z} is independently oxo, halogen, —CN, —NO₂, —OH, —NH₂, C₁-6 alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, each R^{u} is independently oxo, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, 0, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, 0, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{C}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, two R^{u}, together with the carbon atom(s) to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain embodiments, two geminal R^{u}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain aspects, the present disclosure provides compounds of Formula I:

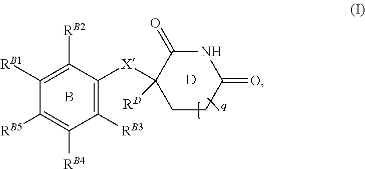

and pharmaceutically acceptable salts, solvates, or stereoisomers thereof,
wherein:
R^{B1}, R^{B2}, R^{B3}, and R^{B4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R^{B5} is

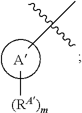

wherein:
Ring A′ is C₄₋₁₂ fused carbocyclyl or 4- to 12-membered fused heterocyclyl comprising 1 or 2 nitrogen atoms;
each R^{A′} is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 10, as valency permits;
or
either R^{B1} and R^{B5} or R^{B4} and R^{B5}, together with the intervening atoms, form Ring A, wherein Ring A is optionally substituted C₃₋₁₂ carbocycle or 3- to 12-membered heterocycle;
X′ is —O— or —NR^{X′}—;
R^{X′} is hydrogen or C₁₋₆ alkyl, wherein the alkyl is optionally substituted with one or more R^{u};
R^{D} is hydrogen, deuterium, C₁₋₆ haloalkyl, or C₁₋₆ alkyl; and
q is an integer from 0 to 2, wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, either R^{B1} and R^{B5} or R^{B4} and R^{B5}, together with the intervening atoms, form Ring A.

In certain embodiments, Ring A is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, and —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl.

In certain embodiments, Ring A is optionally substituted with one or more R^{u}.

In certain embodiments, R^{u} is Rⁱ. In certain embodiments, R^{u} is R⁵. In certain embodiments, R^{u} is R^{X1}. In certain embodiments, R^{u} is Rx2. In certain embodiments, R^{u} is R^{Z1} In certain embodiments, R^{u} is R^{Z2}.

In certain embodiments, Ring A is optionally substituted 7-membered fused heterocycle. In certain embodiments, Ring A is optionally substituted 8-membered fused heterocycle. In certain embodiments, Ring A is optionally substituted 9-membered fused heterocycle. In certain embodiments, Ring A is optionally substituted 10-membered fused heterocycle. In certain embodiments, Ring A is optionally substituted 11-membered fused heterocycle. In certain embodiments, Ring A is optionally substituted 12-membered fused heterocycle.

In certain embodiments, Ring A is optionally substituted 7- to 12-membered fused heterocycle. In certain embodiments, Ring A is optionally substituted 7- to 11-membered fused heterocycle. In certain embodiments, Ring A is optionally substituted 7- to 10-membered fused heterocycle. In certain embodiments, Ring A is optionally substituted 7- to 9-membered fused heterocycle. In certain embodiments, Ring A is optionally substituted 7- to 8-membered fused heterocycle. In certain embodiments, Ring A is optionally substituted 8- to 12-membered fused heterocycle. In certain embodiments, Ring A is optionally substituted 8- to 11-membered fused heterocycle. In certain embodiments, Ring A is optionally substituted 8- to 10-membered fused heterocycle. In certain embodiments, Ring A is optionally substituted 8- to 9-membered fused heterocycle. In certain embodiments, Ring A is optionally substituted 9- to 12-membered fused heterocycle. In certain embodiments, Ring A is optionally substituted 9- to 11-membered fused heterocycle. In certain embodiments, Ring A is optionally substituted 9- to 10-membered fused heterocycle.

In certain embodiments, Ring A is

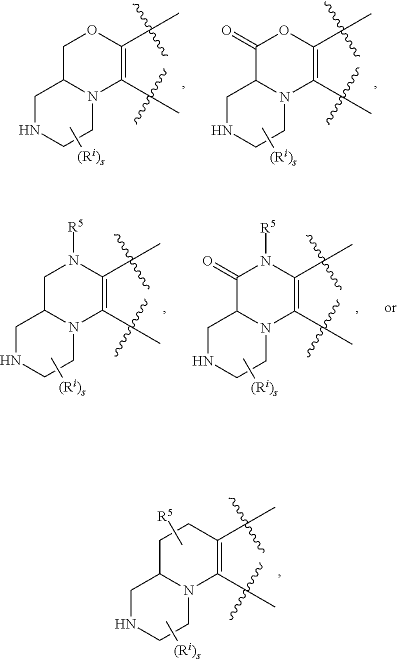

wherein:
R⁵ is hydrogen or C₁₋₆ alkyl, wherein the alkyl is optionally substituted with one or more R^{u};
each Rⁱ is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, the compound of Formula I is a compound of Formula I-1 or I-2

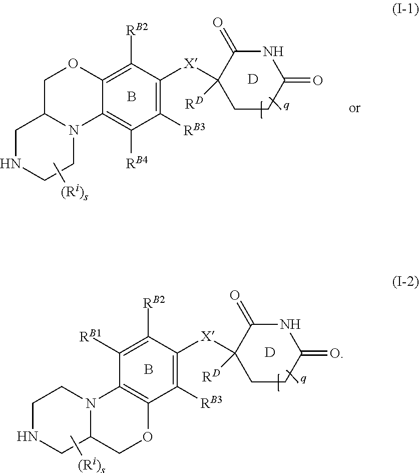

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8.

In certain embodiments, Ring A is optionally substituted 7-membered spiro heterocycle. In certain embodiments, Ring A is optionally substituted 8-membered spiro heterocycle. In certain embodiments, Ring A is optionally substituted 9-membered spiro heterocycle. In certain embodiments, Ring A is optionally substituted 10-membered spiro heterocycle. In certain embodiments, Ring A is optionally substituted 11-membered spiro heterocycle. In certain embodiments, Ring A is optionally substituted 12-membered spiro heterocycle.

In certain embodiments, Ring A is optionally substituted 7- to 12-membered spiro heterocycle. In certain embodiments, Ring A is optionally substituted 7- to 11-membered spiro heterocycle. In certain embodiments, Ring A is optionally substituted 7- to 10-membered spiro heterocycle. In certain embodiments, Ring A is optionally substituted 7- to 9-membered spiro heterocycle. In certain embodiments, Ring A is optionally substituted 7- to 8-membered spiro heterocycle. In certain embodiments, Ring A is optionally substituted 8- to 12-membered spiro heterocycle. In certain embodiments, Ring A is optionally substituted 8- to 11-membered spiro heterocycle. In certain embodiments, Ring A is optionally substituted 8- to 10-membered spiro heterocycle. In certain embodiments, Ring A is optionally substituted 8- to 9-membered spiro heterocycle. In certain embodiments, Ring A is optionally substituted 9- to 12-membered spiro heterocycle. In certain embodiments, Ring A is optionally substituted 9- to 11-membered spiro heterocycle. In certain embodiments, Ring A is optionally substituted 9- to 10-membered spiro heterocycle.

In certain embodiments, Ring A is:

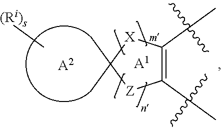

wherein:
Ring A² is C₃₋₈ carbocycle or 3- to 8-membered heterocycle;
each X is independently —C(R^{X1})₂—, —NR^{X2}—, —O—, —S—, —S(═O)—, or —S(═O)₂—;
each Z is independently —C(R^{Z1})₂—, —NR^{Z2}—, —O—, —S—, —S(═O)—, or —S(═O)₂—;
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; two geminal R^{X1} or two geminal R^{Z1} together form oxo; or two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R^{Z2} is independently hydrogen or C₁₋₆ alkyl, wherein the alkyl is optionally substituted with one or more R^{u};
m′ and n′ are independently an integer selected from 0 to 2, wherein one and only one of m′ and n′ is 0;
each Rⁱ is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, Ring A is:

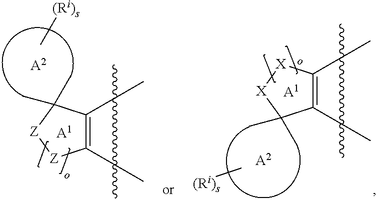

wherein o is 0 or 1.

In certain embodiments, Ring A is:

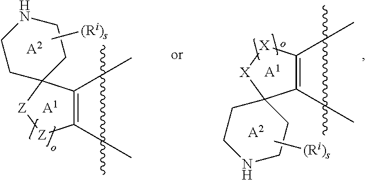

wherein o is 0 or 1.

In certain embodiments, Ring A is:

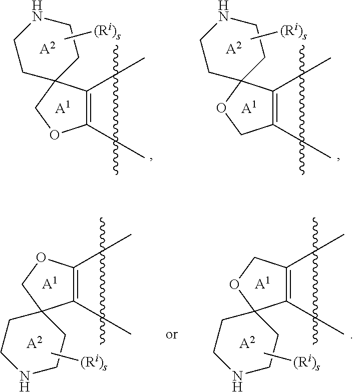

In certain embodiments, o is 0. In certain embodiments, o is 1.

In certain embodiments, the compound of Formula I is a compound of Formula I-3 or 1-4

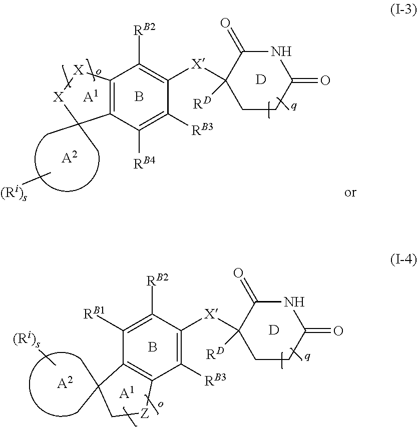

In certain embodiments, each R¹ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, Ring A² is C₃ carbocycle. In certain embodiments, Ring A² is C₄ carbocycle. In certain embodiments, Ring A² is C₅ carbocycle. In certain embodiments, Ring A² is C₆ carbocycle. In certain embodiments, Ring A² is C₇ carbocycle. In certain embodiments, Ring A² is C₈ carbocycle.

In certain embodiments, Ring A² is C₃₋₈ carbocycle. In certain embodiments, Ring A² is C₃₋₇ carbocycle. In certain embodiments, Ring A² is C₃₋₆ carbocycle. In certain embodiments, Ring A² is C₃₋₅ carbocycle. In certain embodiments, Ring A² is C₃₋₄ carbocycle. In certain embodiments, Ring A² is C₄₋₈ carbocycle. In certain embodiments, Ring A² is C₄₋₇ carbocycle.

In certain embodiments, Ring A² is C₄₋₆ carbocycle. In certain embodiments, Ring A² is C₄₋₅ carbocycle. In certain embodiments, Ring A² is C₅₋₈ carbocycle. In certain embodiments, Ring A² is C₅₋₇ carbocycle. In certain embodiments, Ring A² is C₅₋₈ carbocycle. In certain embodiments, Ring A² is C₆₋₈ carbocycle. In certain embodiments, Ring A² is C₆₋₇ carbocycle.

In certain embodiments, Ring A² is 3-membered heterocycle. In certain embodiments, Ring A² is 4-membered heterocycle. In certain embodiments, Ring A² is 5-membered heterocycle. In certain embodiments, Ring A² is 6-membered heterocycle. In certain embodiments, Ring A² is 7-membered heterocycle. In certain embodiments, Ring A² is 8-membered heterocycle.

In certain embodiments, Ring A² is 3- to 8-membered heterocycle. In certain embodiments, Ring A² is 3- to 7-membered heterocycle. In certain embodiments, Ring A² is 3- to 6-membered heterocycle. In certain embodiments, Ring A² is 3- to 5-membered heterocycle. In certain embodiments, Ring A² is 3- to 4-membered heterocycle. In certain embodiments, Ring A² is 4- to 8-membered heterocycle. In certain embodiments, Ring A² is 4- to 7-membered heterocycle. In certain embodiments, Ring A² is 4- to 6-membered heterocycle. In certain embodiments, Ring A² is 4- to 5-membered heterocycle. In certain embodiments, Ring A² is 5- to 8-membered heterocycle. In certain embodiments, Ring A² is 5- to 7-membered heterocycle. In certain embodiments, Ring A² is 5- to 6-membered heterocycle. In certain embodiments, Ring A² is 6- to 8-membered heterocycle. In certain embodiments, Ring A² is 6- to 7-membered heterocycle.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8.

In certain embodiments, R^{B5} is

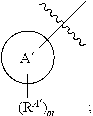

wherein Ring A′ is C₄₋₁₂ fused carbocyclyl or 4- to 12-membered fused heterocyclyl comprising 1 or 2 nitrogen atoms.

In certain embodiments, the compound of Formula I is a compound of Formula I-5

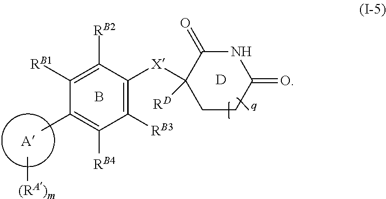

In certain embodiments, R^{B5} is

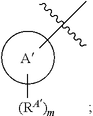

wherein Ring A′ is 4- to 12-membered fused heterocyclyl comprising 1 or 2 nitrogen atoms.

In certain embodiments, Ring A′ is 4-membered fused heterocyclyl comprising 1 or 2 nitrogen atoms. In certain embodiments, Ring A′ is 5-membered fused heterocyclyl comprising 1 or 2 nitrogen atoms. In certain embodiments, Ring A′ is 6-membered fused heterocyclyl comprising 1 or 2 nitrogen atoms. In certain embodiments, Ring A′ is 7-membered fused heterocyclyl comprising 1 or 2 nitrogen atoms. In certain embodiments, Ring A′ is 8-membered fused heterocyclyl comprising 1 or 2 nitrogen atoms. In certain embodiments, Ring A′ is 9-membered fused heterocyclyl comprising 1 or 2 nitrogen atoms. In certain embodiments, Ring A′ is 10-membered fused heterocyclyl comprising 1 or 2 nitrogen atoms. In certain embodiments, Ring A′ is 11-membered fused heterocyclyl comprising 1 or 2 nitrogen atoms. In certain embodiments, Ring A′ is 12-membered fused heterocyclyl comprising 1 or 2 nitrogen atoms.

In certain embodiments, Ring A′ is 4-membered fused heterocyclyl comprising 1 nitrogen atom. In certain embodiments, Ring A′ is 5-membered fused heterocyclyl comprising 1 nitrogen atom. In certain embodiments, Ring A′ is 6-membered fused heterocyclyl comprising 1 nitrogen atom. In certain embodiments, Ring A′ is 7-membered fused heterocyclyl comprising 1 nitrogen atom. In certain embodiments, Ring A′ is 8-membered fused heterocyclyl comprising 1 nitrogen atom. In certain embodiments, Ring A′ is 9-membered fused heterocyclyl comprising 1 nitrogen atom. In certain embodiments, Ring A′ is 10-membered fused heterocyclyl comprising 1 nitrogen atom. In certain embodiments, Ring A′ is 11-membered fused heterocyclyl comprising 1 nitrogen atom. In certain embodiments, Ring A′ is 12-membered fused heterocyclyl comprising 1 nitrogen atom.

In certain embodiments, Ring A′ is 4-membered fused heterocyclyl comprising 2 nitrogen atoms. In certain embodiments, Ring A′ is 5-membered fused heterocyclyl comprising 2 nitrogen atoms. In certain embodiments, Ring A′ is 6-membered fused heterocyclyl comprising 2 nitrogen atoms. In certain embodiments, Ring A′ is 7-membered fused heterocyclyl comprising 2 nitrogen atoms. In certain embodiments, Ring A′ is 8-membered fused heterocyclyl comprising 2 nitrogen atoms. In certain embodiments, Ring A′ is 9-membered fused heterocyclyl comprising 2 nitrogen atoms. In certain embodiments, Ring A′ is 10-membered fused heterocyclyl comprising 2 nitrogen atoms. In certain embodiments, Ring A′ is 11-membered fused heterocyclyl comprising 2 nitrogen atoms. In certain embodiments, Ring A′ is 12-membered fused heterocyclyl comprising 2 nitrogen atoms.

In certain embodiments, Ring A′ is 4- to 12-membered fused heterocyclyl comprising 1 or 2 nitrogen atoms. In certain embodiments, Ring A′ is 4- to 10-membered fused heterocyclyl comprising 1 or 2 nitrogen atoms. In certain embodiments, Ring A′ is 4- to 8-membered fused heterocyclyl comprising 1 or 2 nitrogen atoms. In certain embodiments, Ring A′ is 4- to 6-membered fused heterocyclyl comprising 1 or 2 nitrogen atoms. In certain embodiments, Ring A′ is 6- to 12-membered fused heterocyclyl comprising 1 or 2 nitrogen atoms. In certain embodiments, Ring A′ is 6- to 10-membered fused heterocyclyl comprising 1 or 2 nitrogen atoms. In certain embodiments, Ring A′ is 6- to 8-membered fused heterocyclyl comprising 1 or 2 nitrogen atoms. In certain embodiments, Ring A′ is 8- to 12-membered fused heterocyclyl comprising 1 or 2 nitrogen atoms. In certain embodiments, Ring A′ is 8- to 10-membered fused heterocyclyl comprising 1 or 2 nitrogen atoms.

In certain embodiments, Ring A′ is 4- to 12-membered fused heterocyclyl comprising 1 nitrogen atom. In certain embodiments, Ring A′ is 4- to 10-membered fused heterocyclyl comprising 1 nitrogen atom. In certain embodiments, Ring A′ is 4- to 8-membered fused heterocyclyl comprising 1 nitrogen atom. In certain embodiments, Ring A′ is 4- to 6-membered fused heterocyclyl comprising 1 nitrogen atom. In certain embodiments, Ring A′ is 6- to 12-membered fused heterocyclyl comprising 1 nitrogen atom. In certain embodiments, Ring A′ is 6- to 10-membered fused heterocyclyl comprising 1 nitrogen atom. In certain embodiments, Ring A′ is 6- to 8-membered fused heterocyclyl comprising 1 nitrogen atom. In certain embodiments, Ring A′ is 8- to 12-membered fused heterocyclyl comprising 1 nitrogen atom. In certain embodiments, Ring A′ is 8- to 10-membered fused heterocyclyl comprising 1 nitrogen atom.

In certain embodiments, Ring A′ is 4- to 12-membered fused heterocyclyl comprising 2 nitrogen atoms. In certain embodiments, Ring A′ is 4- to 10-membered fused heterocyclyl comprising 2 nitrogen atoms. In certain embodiments, Ring A′ is 4- to 8-membered fused heterocyclyl comprising 2 nitrogen atoms. In certain embodiments, Ring A′ is 4- to 6-membered fused heterocyclyl comprising 2 nitrogen atoms. In certain embodiments, Ring A′ is 6- to 12-membered fused heterocyclyl comprising 2 nitrogen atoms. In certain embodiments, Ring A′ is 6- to 10-membered fused heterocyclyl comprising 2 nitrogen atoms. In certain embodiments, Ring A′ is 6- to 8-membered fused heterocyclyl comprising 2 nitrogen atoms. In certain embodiments, Ring A′ is 8- to 12-membered fused heterocyclyl comprising 2 nitrogen atoms. In certain embodiments, Ring A′ is 8- to 10-membered fused heterocyclyl comprising 2 nitrogen atoms.

In certain embodiments, R^{B5} is

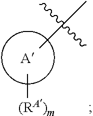

wherein Ring A′ is C₄₋₁₂ fused carbocyclyl.

In certain embodiments, Ring A′ is C₄ fused carbocyclyl. In certain embodiments, Ring A′ is C₅ fused carbocyclyl. In certain embodiments, Ring A′ is C₆ fused carbocyclyl. In certain embodiments, Ring A′ is C₇ fused carbocyclyl. In certain embodiments, Ring A′ is C₈ fused carbocyclyl. In certain embodiments, Ring A′ is C₉ fused carbocyclyl. In certain embodiments, Ring A′ is C₁₀ fused carbocyclyl. In certain embodiments, Ring A′ is C₁₁ fused carbocyclyl. In certain embodiments, Ring A′ is C₁₂ fused carbocyclyl.

In certain embodiments, Ring A′ is C₄₋₁₂ fused carbocyclyl. In certain embodiments, Ring A′ is C₄₋₁₀ fused carbocyclyl. In certain embodiments, Ring A′ is C₄-8 fused carbocyclyl.

In certain embodiments, Ring A′ is C₄₋₆ fused carbocyclyl. In certain embodiments, Ring A′ is C₆₋₁₂ fused carbocyclyl. In certain embodiments, Ring A′ is C₆₋₁₀ fused carbocyclyl. In certain embodiments, Ring A′ is C₆₋₈ fused carbocyclyl. In certain embodiments, Ring A′ is C₈₋₁₂ fused carbocyclyl. In certain embodiments, Ring A′ is C₈₋₁₀ fused carbocyclyl.

In certain embodiments, each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, C₂₋₆ alkenyl, C₂₋₆ alkynyl, aryl, heteroaryl, carbocyclyl, or heterocyclyl is optionally substituted with one or more R^{u}.

In certain embodiments, at least one R^{A′} is halogen.

In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10.

In certain embodiments, m is an integer selected from 0 to 7. In certain embodiments, m is an integer selected from 0 to 5. In certain embodiments, m is an integer selected from 0 to 3. In certain embodiments, m is 0 or 1.

In certain embodiments, R^{B5} is

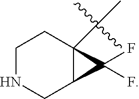 [SMILES: *[C@@]12CCNC[C@@H]1C2(F)F]

In certain embodiments, R^{B1} and R^{B2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{B1} and R^{B2} are independently hydrogen, halogen, or C₁₋₆ alkyl.

In certain embodiments, R^{B3} and R^{B4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{B3} and R^{B4} are independently hydrogen, halogen, or C₁₋₆ alkyl.

In certain embodiments, X′ is O.

In certain embodiments, X′ is —NR^{X′}—.

In certain embodiments, R^{X′} is hydrogen.

In certain embodiments, R^{D} is hydrogen.

In certain embodiments, q is 1.

In certain embodiments, the compound is selected from the compounds in Table 1 and pharmaceutically acceptable salts thereof.

In certain embodiments, the compound is selected from the compounds in Table 1.

TABLE 1
*Note: the compounds below are enantiomerically
pure but the absolute configuration of any
of the stereogenic centers is not determined.

| Com- | |
|---|---|
| pound | |
| No. | Compound Structure |
| 1 | 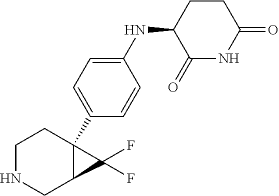 [SMILES: O=C1CC[C@H](Nc2ccc([C@@]34CCNC[C@@H]3C4(F)F)cc2)C(=O)N1] |
| 2 | 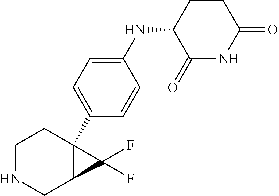 [SMILES: O=C1CC[C@@H](Nc2ccc([C@@]34CCNC[C@@H]3C4(F)F)cc2)C(=O)N1] |
| 3 | 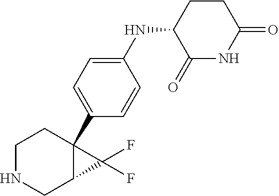 [SMILES: O=C1CC[C@@H](Nc2ccc([C@]34CCNC[C@H]3C4(F)F)cc2)C(=O)N1] |
| 4 | 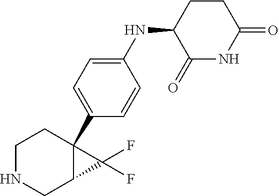 [SMILES: O=C1CC[C@H](Nc2ccc([C@]34CCNC[C@H]3C4(F)F)cc2)C(=O)N1] |
| 5 | 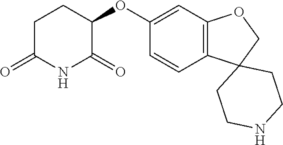 [SMILES: O=C1CC[C@@H](Oc2ccc3c(c2)OCC32CCNCC2)C(=O)N1] |
| 6 | 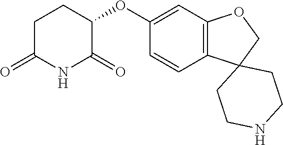 [SMILES: O=C1CC[C@H](Oc2ccc3c(c2)OCC32CCNCC2)C(=O)N1] |
| 7 | 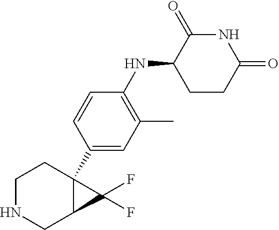 [SMILES: Cc1cc([C@@]23CCNC[C@@H]2C3(F)F)ccc1N[C@@H]1CCC(=O)NC1=O] |
| 8 | 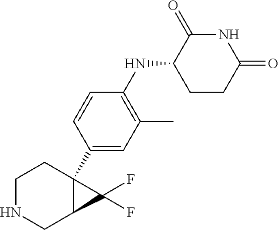 [SMILES: Cc1cc([C@@]23CCNC[C@@H]2C3(F)F)ccc1N[C@H]1CCC(=O)NC1=O] |
| 9 | 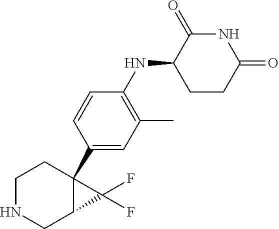 [SMILES: Cc1cc([C@]23CCNC[C@H]2C3(F)F)ccc1N[C@@H]1CCC(=O)NC1=O] |
| 10 | 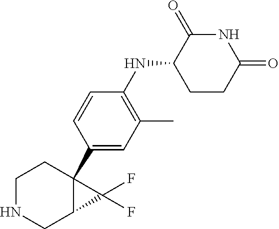 [SMILES: Cc1cc([C@]23CCNC[C@H]2C3(F)F)ccc1N[C@H]1CCC(=O)NC1=O] |
| 11 | 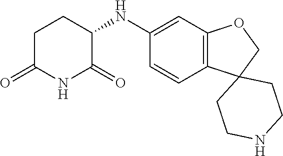 [SMILES: O=C1CC[C@H](Nc2ccc3c(c2)OCC32CCNCC2)C(=O)N1] |
| 12 | 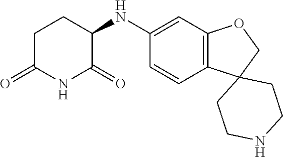 [SMILES: O=C1CC[C@@H](Nc2ccc3c(c2)OCC32CCNCC2)C(=O)N1] |
| 13 | 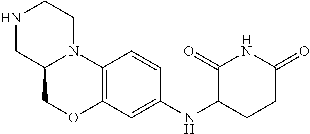 [SMILES: O=C1CCC(Nc2ccc3c(c2)OC[C@H]2CNCCN32)C(=O)N1] |
| 14 | 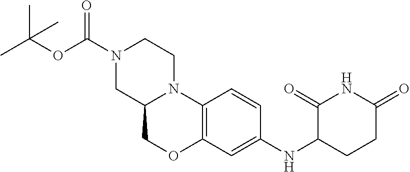 [SMILES: CC(C)(C)OC(=O)N1CCN2c3ccc(NC4CCC(=O)NC4=O)cc3OC[C@H]2C1] |
| 15 | 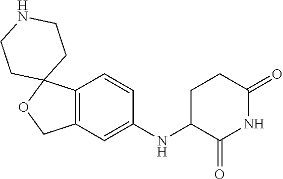 [SMILES: O=C1CCC(Nc2ccc3c(c2)COC32CCNCC2)C(=O)N1] |
| 16 | 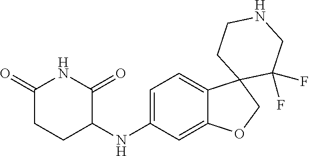 [SMILES: O=C1CCC(Nc2ccc3c(c2)OCC32CCNCC2(F)F)C(=O)N1] |
| 17 | 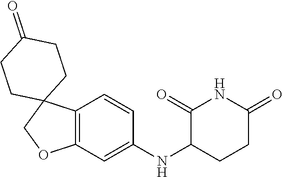 [SMILES: O=C1CCC2(CC1)COc1cc(NC3CCC(=O)NC3=O)ccc12] |
| 18 | 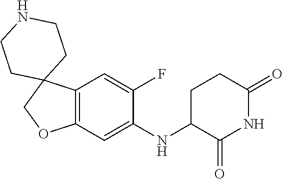 [SMILES: O=C1CCC(Nc2cc3c(cc2F)C2(CCNCC2)CO3)C(=O)N1] |
| 19 | 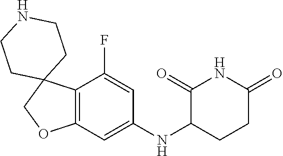 [SMILES: O=C1CCC(Nc2cc(F)c3c(c2)OCC32CCNCC2)C(=O)N1] |

### Bifunctional Degraders

In certain aspects, the present disclosure provides conjugates comprising a compound disclosed herein being connected to a ligand for a protein (e.g., via a linker).

In certain aspects, the present disclosure provides conjugates of Formula II:

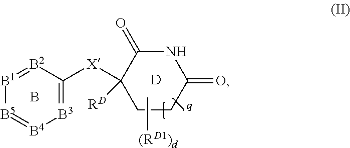

and pharmaceutically acceptable salts, solvates, or stereoisomers thereof,
wherein:
B¹ is N or CR^{B1};
B² is N or CR^{B2};
B³ is N or CR^{B3};
B⁴ is N or CR^{B4};
B⁵ is N or CR^{B5};
wherein:
i) B⁵ is CR^{B5}, either R^{B1} and R^{B5} or R^{B4} and R^{B5}, together with the carbon atoms to which they are bonded, form Ring A attached to -L-T, wherein Ring A is optionally substituted C₅₋₁₆ fused or spiro carbocycle or 5- to 16-membered fused or spiro heterocycle;
R^{B2}, R^{B3}, and R^{B4} or R^{B1}, R^{B2}, and R^{B3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
or
ii) R^{B5} is

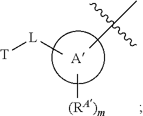

wherein:
Ring A is C₄₋₁₂ fused carbocyclyl or 4- to 12-membered fused heterocyclyl;
each R^{A} is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 10, as valency permits;
R^{B1}, R^{B2}, R^{B3}, and R^{B4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
X′ is —O— or —NR^{X′}—;
R^{X′} is hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u};
each R^{D}1 is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 6;
R^{D} is hydrogen, deuterium, C₁₋₆ haloalkyl, or C₁₋₆ alkyl; and
q is an integer from 0 to 2,
L is a linker; and
T is a ligand for a protein,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, Ring A attached to -L-T is

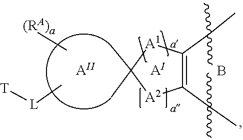

wherein each of the variables is defined herein.

In certain embodiments, Ring A attached to -L-T is

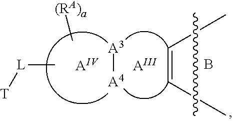

wherein each of the variables is defined herein.

In certain embodiments, Ring A attached to -L-T is

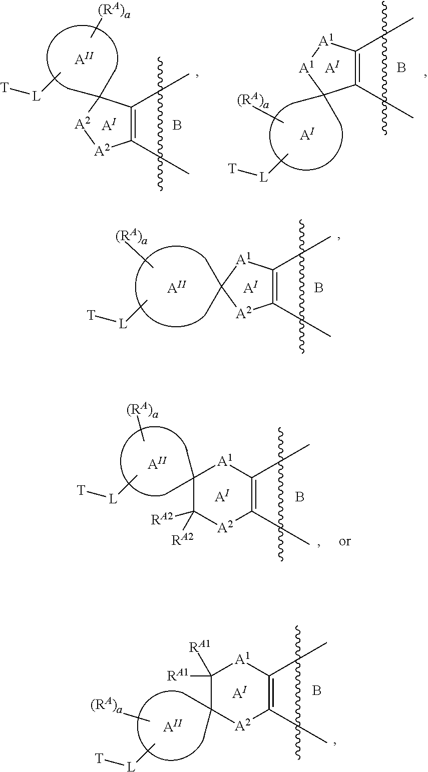

wherein each of the variables is defined herein.

In certain embodiments, Ring A attached to -L-T is

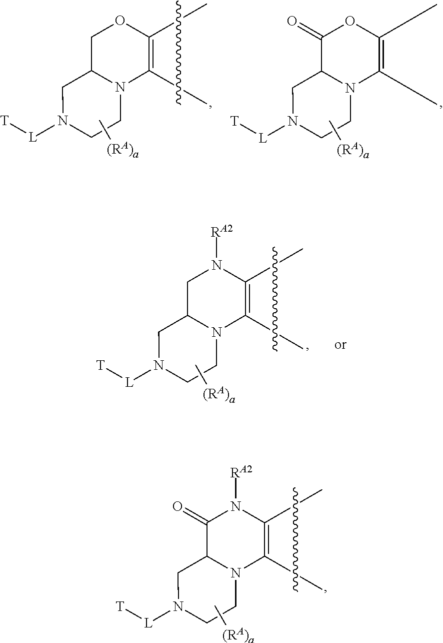

wherein each of the variables is defined herein.

In certain embodiments, the conjugate of Formula II is a conjugate of Formula II-2

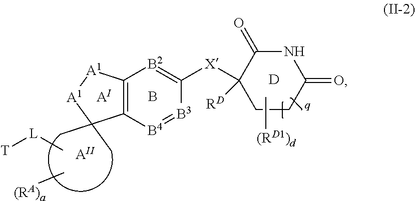

or a pharmaceutically acceptable salt, solvate, or stereoisomer thereof, wherein each of the variables is defined herein.

In certain embodiments, the conjugate of Formula II is a conjugate of Formula II-4

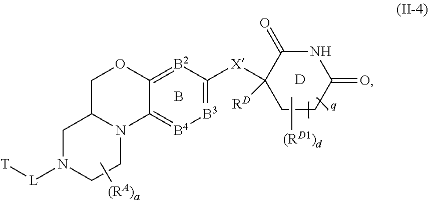

or a pharmaceutically acceptable salt, solvate, or stereoisomer thereof, wherein each of the variables is defined herein.

In certain embodiments, the conjugate of Formula II is a conjugate of Formula II-6

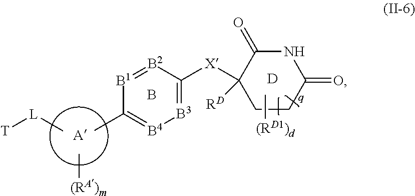

or a pharmaceutically acceptable salt, solvate, or stereoisomer thereof, wherein each of the variables is defined herein.

L, a linker, is a divalent chemical moiety that connects the ligand of a protein with the cereblon ligand disclosed herein. L configures the ligand and the cereblon ligand such that the construct functions as a bifunctional degrader which binds the cereblon ligand and selectively degrads the target protein.

In certain embodiments, L is a linker comprising C₁₋₆ alkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted by one or more R^{u}.

In certain embodiments, L is of Formula II-X

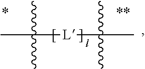

wherein:
* denotes attachment to T and ** denotes attachment to C;
each L′ is independently C₁₋₆ alkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
1 is an integer selected from 0 to 6.

In certain embodiments, each L′ is independently C₁₋₆ alkylene (e.g., methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), and hexylene (—CH₂CH₂CH₂CH₂CH₂CH₂—)), C₂₋₆ alkenylene (e.g., ethenylene (C₂), 1-propenylene (C₃), 2-propenylene (C₃), 1-butenylene (C₄), 2-butenylene (C₄), butadienylene (C₄), pentenylene (C₅), pentadienylene (C₅), or hexenylene (C₆)), C₂₋₆ alkynylene (e.g., ethynylene (C₂), 1-propynylene (C₃), 2-propynylene (C₃), 1-butynylene (C₄), 2-butynylene (C₄), pentynylene (C₅), or hexynylene (C₆)), C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₈), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)), 3- to 12-membered heterocyclylene (e.g., heterocyclylene comprising one or two 3- to 8-membered rings and 1-5 heteroatoms selected from N, O, and S), C₆₋₁₀ arylene (e.g., phenylene or naphthylene), 5- to 10-membered heteroarylene (e.g., heteroarylene comprising one or two 5- or 6-membered rings and 1-5 heteroatoms selected from N, O, and S), —C(═O)—, —C(═O)N(R^{L2})—, —C(═O)O—, —N(R^{L2})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each L′ is independently C₁₋₆ alkylene, C₃₋₁₂ carbocyclylene, 3- to 12-membered heterocyclylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each occurrence of R^{L′} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{L′} is independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, 1 is 0. In certain embodiments, t is 1. In certain embodiments, 1 is 2. In certain embodiments, 1 is 3. In certain embodiments, 1 is 4. In certain embodiments, 1 is 5. In certain embodiments, 1 is 6.

T, a ligand of a protein, is a chemical entity that competitively or non-competitively binds a protein.

In certain embodiments, the protein is B7.1 and B7, TINFR1m, TNFR2, NADPH oxidase, BclIBax and other partners in the apotosis pathway, C₅a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P—glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VC AM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors (AR), adenosine receptors, adenosine kinase and AMP deaminase, purinergic receptors (P2Y1, P2Y2, P2Y4, P2Y6, P2X1-7), farnesyl transferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

In certain embodiments, the protein is an androgen receptor (AR), an estrogen receptor (ER), signal transducer and activator of transcription 3 (STAT3), signal transducer and activator of transcription 5 (STAT5), CREB-binding protein/EP300(E1A) binding protein (CBP/p300), SWI/SNF Related, Matrix Associated, Actin Dependent Regulator Of Chromatin, Subfamily A, Member 2/4 (SMARCA2/4), Kirsten rat sarcoma viral oncogene homolog G12D (KRAS G12D), Src homology region 2-containing protein tyrosine phosphatase 2 (SHP2), bromodomain-containing protein 4 (BRD4), or BRD9.

In certain embodiments, T is a small molecule.

In certain embodiments, T is an antibody.

In certain embodiments, T is a peptide. In certain embodiments, the peptide has about 5 amino acids. In certain embodiments, the peptide has about 10 amino acids. In certain embodiments, the peptide has about 15 amino acids. In certain embodiments, the peptide has about 20 amino acids. In certain embodiments, the peptide has about 25 amino acids. In certain embodiments, the peptide has about 30 amino acids. In certain embodiments, the peptide has about 35 amino acids. In certain embodiments, the peptide has about 40 amino acids. In certain embodiments, the peptide has about 45 amino acids. In certain embodiments, the peptide has about 50 amino acids.

In certain embodiments, T is a ligand for an estrogen receptor. In certain embodiments, T is ligand for an androgen receptor. In certain embodiments, T is ligand for a STAT3 protein.

In certain embodiments, T is an estrogen receptor inhibitor. In certain embodiments, T is an androgen receptor inhibitor. In certain embodiments, T is a STAT3 protein inhibitor.

In certain aspects, the present disclosure provides conjugates of Formula I′:

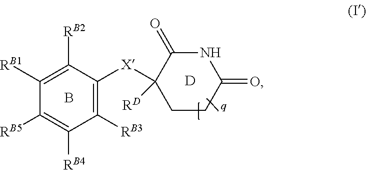

and pharmaceutically acceptable salts, solvates, or stereoisomers thereof,
wherein:
R^{B1}, R^{B2}, R^{B3}, and R^{B4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; R^{B5} is

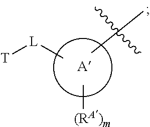

wherein:
Ring A′ is C₄₋₁₂ fused carbocyclyl or 4- to 12-membered fused heterocyclyl comprising 1 or 2 nitrogen atoms;
each R^{A′} is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 10, as valency permits;
or
either R^{B1} and R^{B5} or R^{B4} and R^{B5}, together with the intervening atoms, form Ring A, wherein Ring A is optionally substituted C₃₋₁₂ carbocycle or 3- to 12-membered heterocycle attached to -L-T;
X′ is —O— or —NR^{X′}—;
R^{X′} is hydrogen or C₁₋₆ alkyl, wherein the alkyl is optionally substituted with one or more R^{u};
R^{D} is hydrogen, deuterium, C₁₋₆ haloalkyl, or C₁₋₆ alkyl;
q is an integer from 0 to 2,
L is a linker, and
T is a ligand for a protein,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, either R^{B1} and R^{B5} or R^{B4} and R^{B5}, together with the intervening atoms, form Ring A, wherein Ring A is optionally substituted C₃₋₁₂ carbocycle or 3- to 12-membered heterocycle attached to -L-T.

In certain embodiments, Ring A is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, and —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl.

In certain embodiments, Ring A is optionally substituted with one or more R^{u}.

In certain embodiments, R^{u} is Rⁱ. In certain embodiments, R^{u} is R⁵. In certain embodiments, R^{u} is R^{X1}. In certain embodiments, R^{u} is Rx2. In certain embodiments, R^{u} is R^{Z1}. In certain embodiments, R^{u} is R^{Z2}.

In certain embodiments, Ring A is optionally substituted 7-membered fused heterocycle. In certain embodiments, Ring A is optionally substituted 8-membered fused heterocycle. In certain embodiments, Ring A is optionally substituted 9-membered fused heterocycle. In certain embodiments, Ring A is optionally substituted 10-membered fused heterocycle. In certain embodiments, Ring A is optionally substituted 11-membered fused heterocycle. In certain embodiments, Ring A is optionally substituted 12-membered fused heterocycle.

In certain embodiments, Ring A is optionally substituted 7- to 12-membered fused heterocycle. In certain embodiments, Ring A is optionally substituted 7- to 11-membered fused heterocycle. In certain embodiments, Ring A is optionally substituted 7- to 10-membered fused heterocycle. In certain embodiments, Ring A is optionally substituted 7- to 9-membered fused heterocycle. In certain embodiments, Ring A is optionally substituted 7- to 8-membered fused heterocycle. In certain embodiments, Ring A is optionally substituted 8- to 12-membered fused heterocycle. In certain embodiments, Ring A is optionally substituted 8- to 11-membered fused heterocycle. In certain embodiments, Ring A is optionally substituted 8- to 10-membered fused heterocycle. In certain embodiments, Ring A is optionally substituted 8- to 9-membered fused heterocycle. In certain embodiments, Ring A is optionally substituted 9- to 12-membered fused heterocycle. In certain embodiments, Ring A is optionally substituted 9- to 11-membered fused heterocycle. In certain embodiments, Ring A is optionally substituted 9- to 10-membered fused heterocycle.

In certain embodiments, Ring A is

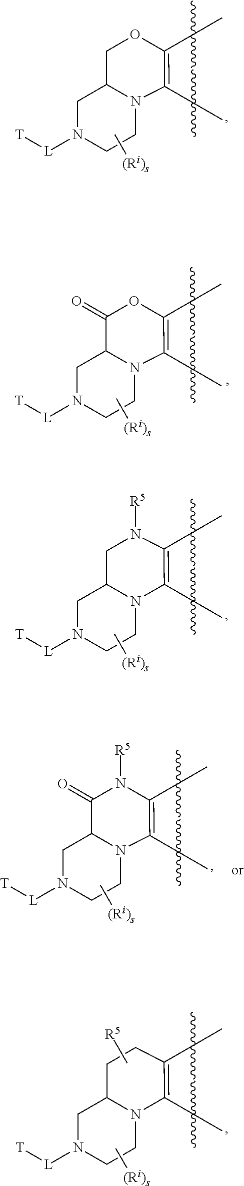

wherein:
R⁵ is hydrogen or C₁₋₆ alkyl, wherein the alkyl is optionally substituted with one or more R^{u}; each R¹ is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, the conjugate of Formula I′ is a conjugate of Formula I′-1 or I′-2

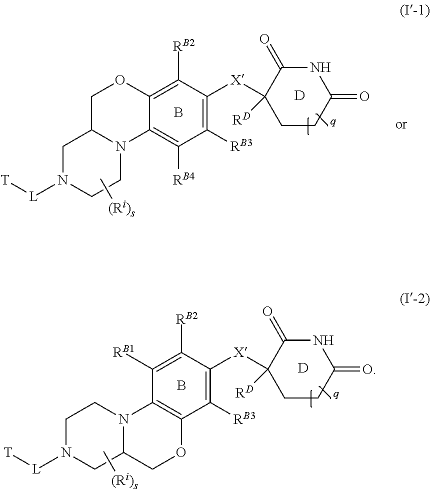

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8.

In certain embodiments, Ring A is optionally substituted 7-membered spiro heterocycle. In certain embodiments, Ring A is optionally substituted 8-membered spiro heterocycle. In certain embodiments, Ring A is optionally substituted 9-membered spiro heterocycle. In certain embodiments, Ring A is optionally substituted 10-membered spiro heterocycle. In certain embodiments, Ring A is optionally substituted 11-membered spiro heterocycle. In certain embodiments, Ring A is optionally substituted 12-membered spiro heterocycle.

In certain embodiments, Ring A is optionally substituted 7- to 12-membered spiro heterocycle. In certain embodiments, Ring A is optionally substituted 7- to 11-membered spiro heterocycle. In certain embodiments, Ring A is optionally substituted 7- to 10-membered spiro heterocycle. In certain embodiments, Ring A is optionally substituted 7- to 9-membered spiro heterocycle. In certain embodiments, Ring A is optionally substituted 7- to 8-membered spiro heterocycle. In certain embodiments, Ring A is optionally substituted 8- to 12-membered spiro heterocycle. In certain embodiments, Ring A is optionally substituted 8- to 11-membered spiro heterocycle. In certain embodiments, Ring A is optionally substituted 8- to 10-membered spiro heterocycle. In certain embodiments, Ring A is optionally substituted 8- to 9-membered spiro heterocycle. In certain embodiments, Ring A is optionally substituted 9- to 12-membered spiro heterocycle. In certain embodiments, Ring A is optionally substituted 9- to 11-membered spiro heterocycle. In certain embodiments, Ring A is optionally substituted 9- to 10-membered spiro heterocycle.

In certain embodiments, Ring A is:

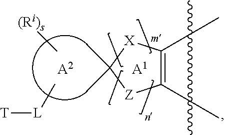

wherein:
Ring A² is C₃₋₈ carbocycle or 3- to 8-membered heterocycle;
each X is independently —C(R^{X1})₂—, —NR^{X2}—, —O—, —S—, —S(═O)—, or —S(═O)₂—; each Z is independently —C(R^{Z1})₂—, —NR^{Z2}—, —O—, —S—, —S(═O)—, or —S(═O)₂—; each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R^{Z2} is independently hydrogen or C₁₋₆ alkyl, wherein the alkyl is optionally substituted with one or more R^{u};
m′ and n′ are independently an integer selected from 0 to 2, wherein one and only one of m′ and n′ is 0;
each Rⁱ is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, Ring A is:

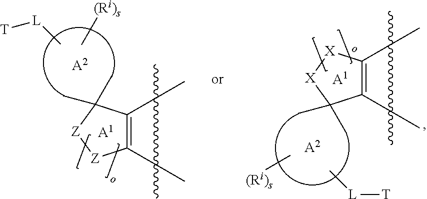

wherein o is 0 or 1.

In certain embodiments, Ring A is:

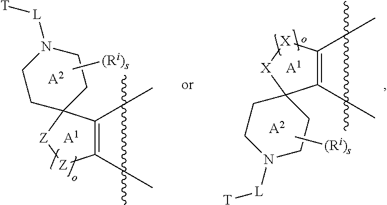

wherein o is 0 or 1.

In certain embodiments, Ring A is:

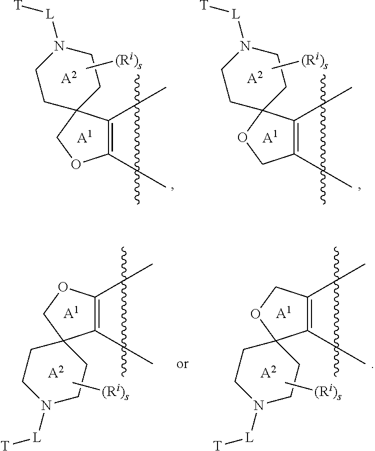

In certain embodiments, o is 0. In certain embodiments, o is 1.

In certain embodiments, the conjugate of Formula I′ is a conjugate of Formula I′-3 or I′-4

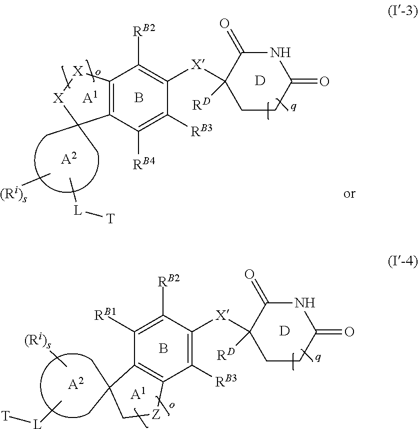

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, Ring A² is C₃ carbocycle. In certain embodiments, Ring A² is C₄ carbocycle. In certain embodiments, Ring A² is C₅ carbocycle. In certain embodiments, Ring A² is C₆ carbocycle. In certain embodiments, Ring A² is C₇ carbocycle. In certain embodiments, Ring A² is C₈ carbocycle.

In certain embodiments, Ring A² is C₃₋₈ carbocycle. In certain embodiments, Ring A² is C₃₋₇ carbocycle. In certain embodiments, Ring A² is C₃₋₆ carbocycle. In certain embodiments, Ring A² is C₃₋₅ carbocycle. In certain embodiments, Ring A² is C₃₋₄ carbocycle. In certain embodiments, Ring A² is C₄₋₈ carbocycle. In certain embodiments, Ring A² is C₄₋₇ carbocycle.

In certain embodiments, Ring A² is C₄₋₆ carbocycle. In certain embodiments, Ring A² is C₄₋₅ carbocycle. In certain embodiments, Ring A² is C₅₋₈ carbocycle. In certain embodiments, Ring A² is C₅₋₇ carbocycle. In certain embodiments, Ring A² is C₅₋₈ carbocycle. In certain embodiments, Ring A² is C₆₋₈ carbocycle. In certain embodiments, Ring A² is C₆₋₇ carbocycle.

In certain embodiments, Ring A² is 3-membered heterocycle. In certain embodiments, Ring A² is 4-membered heterocycle. In certain embodiments, Ring A² is 5-membered heterocycle. In certain embodiments, Ring A² is 6-membered heterocycle. In certain embodiments, Ring A² is 7-membered heterocycle. In certain embodiments, Ring A² is 8-membered heterocycle.

In certain embodiments, Ring A² is 3- to 8-membered heterocycle. In certain embodiments, Ring A² is 3- to 7-membered heterocycle. In certain embodiments, Ring A² is 3- to 6-membered heterocycle. In certain embodiments, Ring A² is 3- to 5-membered heterocycle. In certain embodiments, Ring A² is 3- to 4-membered heterocycle. In certain embodiments, Ring A² is 4- to 8-membered heterocycle. In certain embodiments, Ring A² is 4- to 7-membered heterocycle. In certain embodiments, Ring A² is 4- to 6-membered heterocycle. In certain embodiments, Ring A² is 4- to 5-membered heterocycle. In certain embodiments, Ring A² is 5- to 8-membered heterocycle. In certain embodiments, Ring A² is 5- to 7-membered heterocycle. In certain embodiments, Ring A² is 5- to 6-membered heterocycle. In certain embodiments, Ring A² is 6- to 8-membered heterocycle. In certain embodiments, Ring A² is 6- to 7-membered heterocycle.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8.

In certain embodiments, R^{B5} is

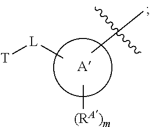

wherein Ring A is C₄₋₁₂ fused carbocyclyl or 4- to 12-membered fused heterocyclyl comprising 1 or 2 nitrogen atoms.

In certain embodiments, the conjugate of Formula I′ is a conjugate of Formula I′-5

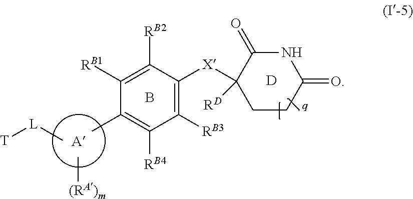

In certain embodiments, R^{B5} is

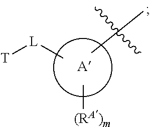

wherein Ring A′ is 4- to 12-membered fused heterocyclyl comprising 1 or 2 nitrogen atoms.

In certain embodiments, Ring A′ is 4-membered fused heterocyclyl comprising 1 or 2 nitrogen atoms. In certain embodiments, Ring A′ is 5-membered fused heterocyclyl comprising 1 or 2 nitrogen atoms. In certain embodiments, Ring A′ is 6-membered fused heterocyclyl comprising 1 or 2 nitrogen atoms. In certain embodiments, Ring A′ is 7-membered fused heterocyclyl comprising 1 or 2 nitrogen atoms. In certain embodiments, Ring A′ is 8-membered fused heterocyclyl comprising 1 or 2 nitrogen atoms. In certain embodiments, Ring A′ is 9-membered fused heterocyclyl comprising 1 or 2 nitrogen atoms. In certain embodiments, Ring A′ is 10-membered fused heterocyclyl comprising 1 or 2 nitrogen atoms. In certain embodiments, Ring A′ is 11-membered fused heterocyclyl comprising 1 or 2 nitrogen atoms.

In certain embodiments, Ring A′ is 12-membered fused heterocyclyl comprising 1 or 2 nitrogen atoms.

In certain embodiments, Ring A′ is 4-membered fused heterocyclyl comprising 1 nitrogen atom. In certain embodiments, Ring A′ is 5-membered fused heterocyclyl comprising 1 nitrogen atom. In certain embodiments, Ring A′ is 6-membered fused heterocyclyl comprising 1 nitrogen atom. In certain embodiments, Ring A′ is 7-membered fused heterocyclyl comprising 1 nitrogen atom. In certain embodiments, Ring A′ is 8-membered fused heterocyclyl comprising 1 nitrogen atom. In certain embodiments, Ring A′ is 9-membered fused heterocyclyl comprising 1 nitrogen atom. In certain embodiments, Ring A′ is 10-membered fused heterocyclyl comprising 1 nitrogen atom. In certain embodiments, Ring A′ is 11-membered fused heterocyclyl comprising 1 nitrogen atom. In certain embodiments, Ring A′ is 12-membered fused heterocyclyl comprising 1 nitrogen atom.

In certain embodiments, Ring A′ is 4-membered fused heterocyclyl comprising 2 nitrogen atoms. In certain embodiments, Ring A′ is 5-membered fused heterocyclyl comprising 2 nitrogen atoms. In certain embodiments, Ring A′ is 6-membered fused heterocyclyl comprising 2 nitrogen atoms. In certain embodiments, Ring A′ is 7-membered fused heterocyclyl comprising 2 nitrogen atoms. In certain embodiments, Ring A′ is 8-membered fused heterocyclyl comprising 2 nitrogen atoms. In certain embodiments, Ring A′ is 9-membered fused heterocyclyl comprising 2 nitrogen atoms. In certain embodiments, Ring A′ is 10-membered fused heterocyclyl comprising 2 nitrogen atoms. In certain embodiments, Ring A′ is 11-membered fused heterocyclyl comprising 2 nitrogen atoms. In certain embodiments, Ring A′ is 12-membered fused heterocyclyl comprising 2 nitrogen atoms.

In certain embodiments, Ring A′ is 4- to 12-membered fused heterocyclyl comprising 1 or 2 nitrogen atoms. In certain embodiments, Ring A′ is 4- to 10-membered fused heterocyclyl comprising 1 or 2 nitrogen atoms. In certain embodiments, Ring A′ is 4- to 8-membered fused heterocyclyl comprising 1 or 2 nitrogen atoms. In certain embodiments, Ring A′ is 4- to 6-membered fused heterocyclyl comprising 1 or 2 nitrogen atoms. In certain embodiments, Ring A′ is 6- to 12-membered fused heterocyclyl comprising 1 or 2 nitrogen atoms. In certain embodiments, Ring A′ is 6- to 10-membered fused heterocyclyl comprising 1 or 2 nitrogen atoms. In certain embodiments, Ring A′ is 6- to 8-membered fused heterocyclyl comprising 1 or 2 nitrogen atoms. In certain embodiments, Ring A′ is 8- to 12-membered fused heterocyclyl comprising 1 or 2 nitrogen atoms. In certain embodiments, Ring A′ is 8- to 10-membered fused heterocyclyl comprising 1 or 2 nitrogen atoms.

In certain embodiments, Ring A′ is 4- to 12-membered fused heterocyclyl comprising 1 nitrogen atom. In certain embodiments, Ring A′ is 4- to 10-membered fused heterocyclyl comprising 1 nitrogen atom. In certain embodiments, Ring A′ is 4- to 8-membered fused heterocyclyl comprising 1 nitrogen atom. In certain embodiments, Ring A′ is 4- to 6-membered fused heterocyclyl comprising 1 nitrogen atom. In certain embodiments, Ring A′ is 6- to 12-membered fused heterocyclyl comprising 1 nitrogen atom. In certain embodiments, Ring A′ is 6- to 10-membered fused heterocyclyl comprising 1 nitrogen atom. In certain embodiments, Ring A′ is 6- to 8-membered fused heterocyclyl comprising 1 nitrogen atom. In certain embodiments, Ring A′ is 8- to 12-membered fused heterocyclyl comprising 1 nitrogen atom. In certain embodiments, Ring A′ is 8- to 10-membered fused heterocyclyl comprising 1 nitrogen atom.

In certain embodiments, Ring A′ is 4- to 12-membered fused heterocyclyl comprising 2 nitrogen atoms. In certain embodiments, Ring A′ is 4- to 10-membered fused heterocyclyl comprising 2 nitrogen atoms. In certain embodiments, Ring A′ is 4- to 8-membered fused heterocyclyl comprising 2 nitrogen atoms. In certain embodiments, Ring A′ is 4- to 6-membered fused heterocyclyl comprising 2 nitrogen atoms. In certain embodiments, Ring A′ is 6- to 12-membered fused heterocyclyl comprising 2 nitrogen atoms. In certain embodiments, Ring A′ is 6- to 10-membered fused heterocyclyl comprising 2 nitrogen atoms. In certain embodiments, Ring A′ is 6- to 8-membered fused heterocyclyl comprising 2 nitrogen atoms. In certain embodiments, Ring A′ is 8- to 12-membered fused heterocyclyl comprising 2 nitrogen atoms. In certain embodiments, Ring A′ is 8- to 10-membered fused heterocyclyl comprising 2 nitrogen atoms.

In certain embodiments, R^{B5} is

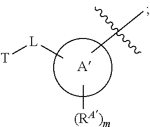

wherein Ring A′ is C₄₋₁₂ fused carbocyclyl.

In certain embodiments, Ring A′ is C₄ fused carbocyclyl. In certain embodiments, Ring A′ is C₅ fused carbocyclyl. In certain embodiments, Ring A′ is C₆ fused carbocyclyl. In certain embodiments, Ring A′ is C₇ fused carbocyclyl. In certain embodiments, Ring A′ is C₈ fused carbocyclyl. In certain embodiments, Ring A′ is C₉ fused carbocyclyl. In certain embodiments, Ring A′ is C₁₀ fused carbocyclyl. In certain embodiments, Ring A′ is C₁₁ fused carbocyclyl. In certain embodiments, Ring A′ is C₁₂ fused carbocyclyl.

In certain embodiments, Ring A′ is C₄₋₁₂ fused carbocyclyl. In certain embodiments, Ring A′ is C₄₋₁₀ fused carbocyclyl. In certain embodiments, Ring A′ is C₄-8 fused carbocyclyl.

In certain embodiments, Ring A′ is C₄₋₆ fused carbocyclyl. In certain embodiments, Ring A′ is C₆₋₁₂ fused carbocyclyl. In certain embodiments, Ring A′ is C₆₋₁₀ fused carbocyclyl. In certain embodiments, Ring A′ is C₆₋₈ fused carbocyclyl. In certain embodiments, Ring A′ is C₈₋₁₂ fused carbocyclyl. In certain embodiments, Ring A′ is C₈₋₁₀ fused carbocyclyl.

In certain embodiments, each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, or 3- to 6-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, each R^{A′} is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};

In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10.

In certain embodiments, m is an integer selected from 0 to 7. In certain embodiments, m is an integer selected from 0 to 5. In certain embodiments, m is an integer selected from 0 to 3. In certain embodiments, m is 0 or 1.

In certain embodiments, R^{B5} is

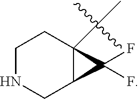 [SMILES: *[C@@]12CCNC[C@@H]1C2(F)F]

In certain embodiments, R^{B1} and R^{B2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{B1} and R^{B2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B3} and R^{B4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{B3} and R^{B4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, X′ is 0.

In certain embodiments, X′ is —NR^{X′}—.

In certain embodiments, R^{X′} is hydrogen.

In certain embodiments, R^{D} is hydrogen.

In certain embodiments, q is 0. In certain embodiments, q is 1. In certain embodiments, q is 2.

L, a linker, is a divalent chemical moiety that connects the ligand of a protein with the cereblon ligand disclosed herein. L configures the ligand and the cereblon ligand such that the construct functions as a bifunctional degrader which binds the cereblon ligand and selectively degrads the target protein.

In certain embodiments, L is a linker comprising 6- to 10-membered heteroarylene, C₆₋₁₀ arylene, C₃₋₁₂ membered carbocyclylene, or 3- to 12-membered heterocyclylene, wherein the arylene, heteroarylene, carbocyclylene, or heterocyclylene is optionally substituted by one or more R^{u}, and is directly attached to T.

In certain embodiments, L is of formula

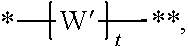

wherein:
* denotes attachment to T and ** denotes attachment to the cereblon ligand;
each —W′—is independently C₁₋₃ alkylene, C₂ alkenylene, C₂ alkynylene, C₃₋₁₂ carbocycylene, 3- to 12-membered heterocyclylene, C₆₋₁₀ arylene, 5- to 10-membered heteroarylene, —C(═O)—, —N(R^{L})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L} is independently hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkenyl, alkynyl, carbocyclyl, heterocyclyl, aryl, or heteroaryl is optionally substituted with one or more R^{u}; and
t is an integer selected from 1 to 15.

In certain embodiments, L is of formula

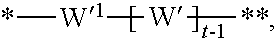

wherein:
W′¹ is 6- to 10-membered heteroarylene, C₆₋₁₀ arylene, C₃₋₁₂ membered carbocyclylene, or 3- to 12-membered heterocyclylene, wherein the arylene, heteroarylene, carbocyclylene, or heterocyclylene is optionally substituted by one or more R^{u}; and
each —W′—is independently C₁₋₃ alkylene, —C(═O)—, —N(R^{L})—, —O—, C₃₋₁₂ carbocycylene, or 3- to 12-membered heterocyclylene, wherein the alkylene, carbocyclylene, or heterocyclylene is optionally substituted with one or more R^{u}.

In certain embodiments, L is of Formula:

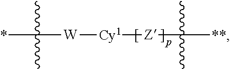

wherein:
W is absent; or
W is C₁₋₃ alkylene, —O—, —NR^{W}—, or —(C═O)—, wherein the alkylene is optionally substituted by one or more R^{u};
Cy¹ is absent; or
Cy¹ is 6-membered heteroarylene, C₆ arylene, C₃₋₁₂ membered carbocyclylene, or 3- to 12-membered heterocyclylene, wherein the arylene, heteroarylene, carbocyclylene, or heterocyclylene is optionally substituted by one or more R^{u};
Z′ is absent; or
each Z′ is independently C₁₋₃ alkylene, —O—, —NR^{W}—, —(C═O)—, C₃₋₁₂ membered carbocyclylene, or 3- to 12-membered heterocyclylene, wherein the alkylene, carbocyclylene, or heterocyclylene is optionally substituted by one or more R^{u};
R^{W} is hydrogen or C₁₋₆ alkyl optionally substituted with one or more R^{u}; and
p is an integer selected from 0 to 8.

T, a ligand of a protein, is a chemical entity that competitively or non-competitively binds a protein.

In certain embodiments, the protein is B7.1 and B7, TINFR1m, TNFR2, NADPH oxidase, BelIBax and other partners in the apotosis pathway, C₅a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P—glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VC AM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors (AR), adenosine receptors, adenosine kinase and AMP deaminase, purinergic receptors (P2Y1, P2Y2, P2Y4, P2Y6, P2X1-7), farnesyl transferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

In certain embodiments, the protein is an androgen receptor (AR), an estrogen receptor (ER), signal transducer and activator of transcription 3 (STAT3), signal transducer and activator of transcription 5 (STAT5), CREB-binding protein/EP300(E1A) binding protein (CBP/p300), SMARCA2/4, Kirsten rat sarcoma viral oncogene homolog G12D (KRAS G12D), Src homology region 2-containing protein tyrosine phosphatase 2 (SHP2), or bromodomain-containing protein 4 (BRD4).

In certain embodiments, T is a small molecule.

In certain embodiments, T is an antibody.

In certain embodiments, T is a peptide. In certain embodiments, the peptide has about 5 amino acids. In certain embodiments, the peptide has about 10 amino acids. In certain embodiments, the peptide has about 15 amino acids. In certain embodiments, the peptide has about 20 amino acids. In certain embodiments, the peptide has about 25 amino acids. In certain embodiments, the peptide has about 30 amino acids. In certain embodiments, the peptide has about 35 amino acids. In certain embodiments, the peptide has about 40 amino acids. In certain embodiments, the peptide has about 45 amino acids. In certain embodiments, the peptide has about 50 amino acids.

In certain embodiments, T is a ligand for an estrogen receptor. In certain embodiments, T is ligand for an androgen receptor. In certain embodiments, T is ligand for a STAT3 protein.

In certain embodiments, T is an estrogen receptor inhibitor. In certain embodiments, T is an androgen receptor inhibitor. In certain embodiments, T is a STAT3 protein inhibitor.

The compounds of the present disclosure possess advantageous characteristics, as compared to known compounds, such as known cereblon-binding agents or known degraders comprising such cereblon-binding agents. For example, the compounds of the present disclosure display more potent cereblon-binding activity or more potent degradation activity against certain proteins, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction).

These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Forms of Compounds Disclosed Herein

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefor react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, 7-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the invention.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present invention.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers/Stereoisomers

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the invention.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the invention.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. All tautomeric forms of the compounds disclosed herein are contemplated and are within the scope of the invention. The exact ratio of the tautomers depends on several factors, including temperature, solvent, and pH.

### Pharmaceutical Compositions

In certain embodiments, the compound described herein is administered as a pure chemical. In certain embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In certain embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In certain embodiments, the pharmaceutical composition is formulated for intravenous injection, oral administration, inhalation, nasal administration, topical administration, or ophthalmic administration. In certain embodiments, the pharmaceutical composition is formulated for oral administration. In certain embodiments, the pharmaceutical composition is formulated for intravenous injection. In certain embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In certain embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Scheme

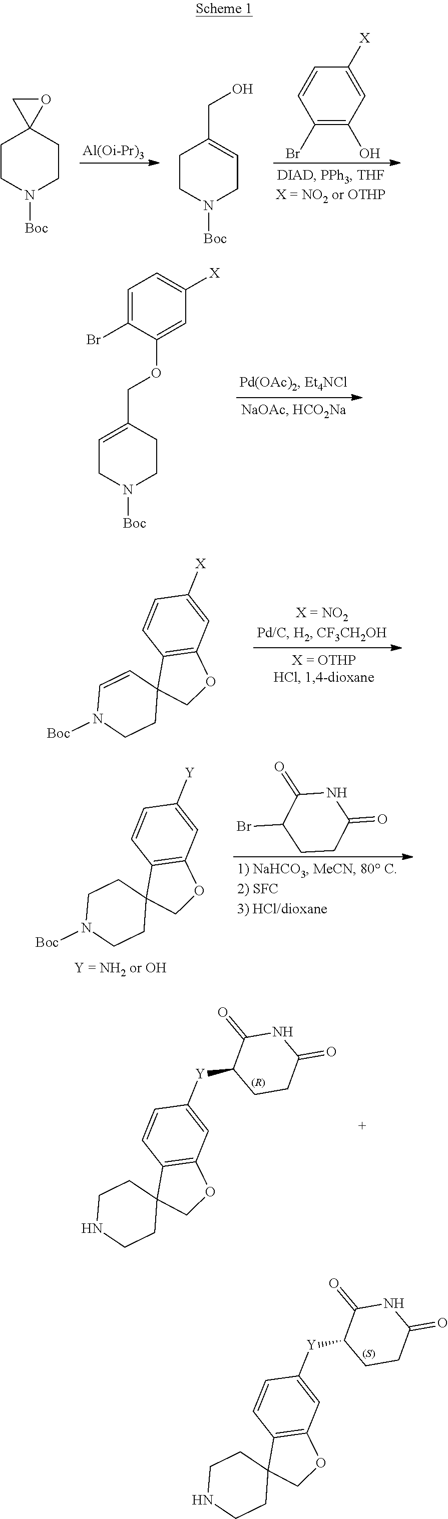

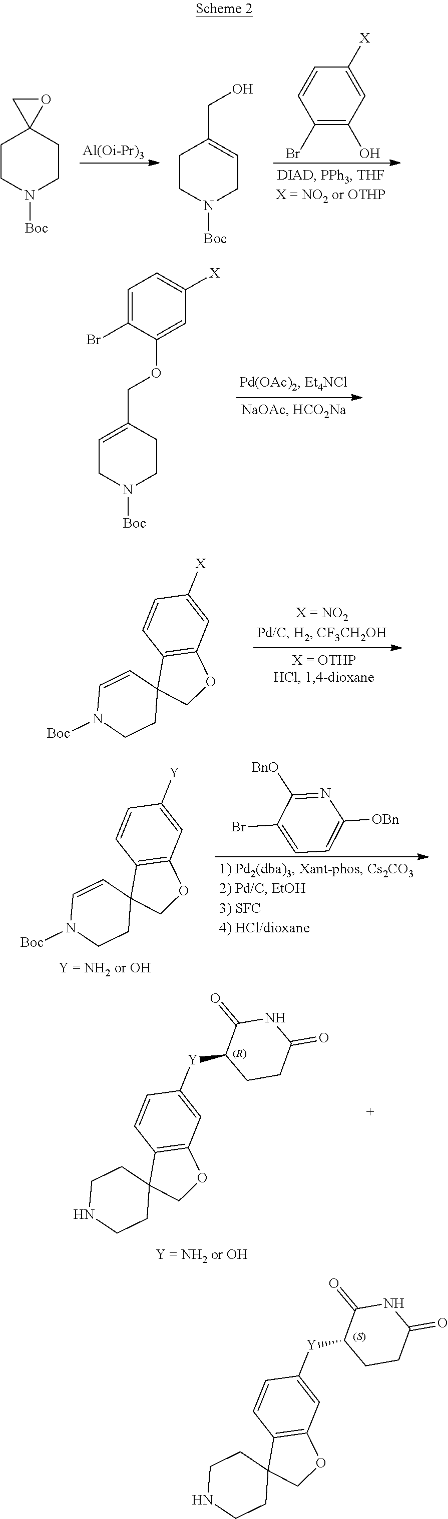

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Comwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 m 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present disclosure can be assessed with methods and assays known in the art.

The CRBN−DDB1 binding potency of the present disclosure was determined using HTRF assay technology (Perkin Elmer). Compounds are serially diluted and are transferred multi-well plate. The reaction was conducted with addition of His tagged (e.g., CRBN+DDB-DLS7+CXU4) followed by addition of fluorescent probe (e.g., Cy5-labeled Thalidomide), and MAb Anti-6HIS Tb cryptate Gold in the assay buffer. After certain period of incubation at room temperature, the HTRF signals were read on a reader.

### Methods of Use

“CRBN E3 ubiquitin ligase protein complex” is art recognized and refers to an association of proteins in which CRBN, a 442-amino acid protein, forms a Cullin-4-RING E3 ubiquitin ligase (CRL4) complex and interacts with the adaptor protein damaged DNA-binding protein 1 (DDB1), Cullin-4A (CUL4A), and regulator of cullins 1 (ROC1). Within the CRL4 complex, CRBN acts as a substrate-specificity receptor.

In certain aspects, provided herein are methods of binding cereblon E3 ubiquitin ligase protein complex in a subject or biological sample comprising administering a compound described herein to the subject or contacting the biological sample with a compound described herein.

In certain aspects, provided herein are uses of a compound described herein in the manufacture of a medicament for binding cereblon E3 ubiquitin ligase protein complex in a subject or biological sample.

In certain aspects, provided herein are compounds described herein for use in binding cereblon E3 ubiquitin ligase protein complex in a subject or biological sample.

In certain aspects, provided herein are methods of degrading a protein in a subject or biological sample comprising administering a conjugate described herein to the subject or contacting the biological sample with a compound described herein.

In certain aspects, provided herein are uses of a conjugate described herein in the manufacture of a medicament for degrading a protein in a subject or biological sample.

In certain aspects, provided herein are conjugates described herein for use in degrading a protein in a subject or biological sample.

In certain embodiments, the protein is an androgen receptor (AR), an estrogen receptor (ER), signal transducer and activator of transcription 3 (STAT3), STAT5, CREB-binding protein/EP300(E1A) binding protein (CBP/p300), SWI/SNF Related, Matrix Associated, Actin Dependent Regulator Of Chromatin, Subfamily A, Member 2/4 (SMARCA2/4), Kirsten rat sarcoma viral oncogene homolog G12D (KRAS G12D), Src homology region 2-containing protein tyrosine phosphatase 2 (SHP2), bromodomain-containing protein 4 (BRD4), or BRD9.

In certain aspects, provided herein are methods of treating or preventing a disease or disorder a subject in need thereof, comprising administering to the subject a conjugate described herein.

In certain aspects, provided herein are uses of a conjugate described herein in the manufacture of a medicament for treating or preventing a disease or disorder in a subject in need thereof.

In certain aspects, provided herein are conjugates described herein for use in treating or preventing a disease or disorder in a subject in need thereof.

In certain embodiments, the disease or disorder is an estrogen receptor-mediated disease or disorder, an androgen receptor-mediated disease or disorder, or a STAT1/3 protein-mediated disease or disorder.

In certain embodiments, the protein is an androgen receptor (AR)-mediated disease or disorder, an estrogen receptor (ER)-mediated disease or disorder, signal transducer and activator of transcription 3-mediated disease or disorder (STAT3-mediated disease or disorder), STAT5-mediated disease or disorder, CREB-binding protein/EP300(E1A) binding protein-mediated disease or disorder (CBP/p300)-mediated disease or disorder, SWI/SNF Related, Matrix Associated, Actin Dependent Regulator Of Chromatin, Subfamily A, Member 2/4-mediated disease or disorder (SMARCA2/4-mediated disease or disorder), Kirsten rat sarcoma viral oncogene homolog G12D-mediated disease or disorder (KRAS G12D-mediated disease or disorder), Src homology region 2-containing protein tyrosine phosphatase 2-mediated disease or disorder (SHP2-mediated disease or disorder), bromodomain-containing protein 4-mediated disease or disorder (BRD4-mediated disease or disorder), or BRD9-mediated disease or disorder.

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75′ Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5′ Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋ₛ alkyl”). In certain embodiments, an alkyl group has 1 to 7 carbon atoms (“C₁₋₇ alkyl”).

In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkelene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂-9 alkenyl”). In certain embodiments, an alkenyl group has 2 to 8 carbon atoms (“C₂₋₅ alkenyl”).

In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”).

In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl).

Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In certain embodiments, a heteroalkyl group is a saturated group having 1 to 9 carbon atoms and 1, 2, 3, or 4 heteroatoms (“heteroC₁₋₉ alkyl”). In certain embodiments, a heteroalkyl group is a saturated group having 1 to 8 carbon atoms and 1, 2, 3, or 4 heteroatoms (“heteroC₁₋₈ alkyl”). In certain embodiments, a heteroalkyl group is a saturated group having 1 to 7 carbon atoms and 1, 2, 3, or 4 heteroatoms (“heteroC₁₋₇ alkyl”). In certain embodiments, a heteroalkyl group is a group having 1 to 6 carbon atoms and 1, 2, or 3 heteroatoms (“heteroC₁₋₆ alkyl”). In certain embodiments, a heteroalkyl group is a saturated group having 1 to 5 carbon atoms and 1 or 2 heteroatoms (“heteroC₁₋₅ alkyl”). In certain embodiments, a heteroalkyl group is a saturated group having 1 to 4 carbon atoms and/or 2 heteroatoms (“heteroC₁₋₄ alkyl”). In certain embodiments, a heteroalkyl group is a saturated group having 1 to 3 carbon atoms and 1 heteroatom (“heteroC₁₋₃ alkyl”). In certain embodiments, a heteroalkyl group is a saturated group having 1 to 2 carbon atoms and 1 heteroatom (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment.

In certain embodiments, a heteroalkenyl group refers to a group having from 2 to 10 carbon atoms, at least one double bond, and 1, 2, 3, or 4 heteroatoms (“heteroC₂₋₁₀ alkenyl”). In certain embodiments, a heteroalkenyl group has 2 to 9 carbon atoms at least one double bond, and 1, 2, 3, or 4 heteroatoms (“heteroC₂₋₉ alkenyl”). In certain embodiments, a heteroalkenyl group has 2 to 8 carbon atoms, at least one double bond, and 1, 2, 3, or 4 heteroatoms (“heteroC₂₋₈ alkenyl”). In certain embodiments, a heteroalkenyl group has 2 to 7 carbon atoms, at least one double bond, and 1, 2, 3, or 4 heteroatoms (“heteroC₂₋₇ alkenyl”). In certain embodiments, a heteroalkenyl group has 2 to 6 carbon atoms, at least one double bond, and 1, 2, or 3 heteroatoms (“heteroC₂₋₆ alkenyl”). In certain embodiments, a heteroalkenyl group has 2 to 5 carbon atoms, at least one double bond, and 1 or 2 heteroatoms (“heteroC₂₋₅ alkenyl”). In certain embodiments, a heteroalkenyl group has 2 to 4 carbon atoms, at least one double bond, and lor 2 heteroatoms (“heteroC₂₋₄ alkenyl”). In certain embodiments, a heteroalkenyl group has 2 to 3 carbon atoms, at least one double bond, and 1 heteroatom (“heteroC₂₋₃ alkenyl”). In certain embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment.

In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In certain embodiments, a heteroalkynyl group has 2 to 9 carbon atoms, at least one triple bond, and 1, 2, 3, or 4 heteroatoms (“heteroC₂₋₉ alkynyl”). In certain embodiments, a heteroalkynyl group has 2 to 8 carbon atoms, at least one triple bond, and 1, 2, 3, or 4 heteroatoms (“heteroC₂₋₈ alkynyl”).

In certain embodiments, a heteroalkynyl group has 2 to 7 carbon atoms, at least one triple bond, and 1, 2, 3, or 4 heteroatoms (“heteroC₂₋₇ alkynyl”). In certain embodiments, a heteroalkynyl group has 2 to 6 carbon atoms, at least one triple bond, and 1, 2, or 3 heteroatoms (“heteroC₂₋₆ alkynyl”). In certain embodiments, a heteroalkynyl group has 2 to 5 carbon atoms, at least one triple bond, and 1 or 2 heteroatoms (“heteroC₂₋₅ alkynyl”). In certain embodiments, a heteroalkynyl group has 2 to 4 carbon atoms, at least one triple bond, and lor 2 heteroatoms (“heteroC₂₋₄ alkynyl”). In certain embodiments, a heteroalkynyl group has 2 to 3 carbon atoms, at least one triple bond, and 1 heteroatom (“heteroC₂₋₃ alkynyl”). In certain embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”).

In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl, as defined herein, and refers to an optionally substituted alkyl group substituted by an optionally substituted aryl group.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur.

In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl, as defined herein, and refers to an optionally substituted alkyl group substituted by an optionally substituted heteroaryl group.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share one common bond with, one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or or “bridged carbocycle” refers to ring systems wherein the carbocyclyl group, as defined above, form bridged structure with, i.e., share more than one atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the bridged rings. When substitution is indicated, unless otherwise specified, substitution can occur on any of the carbocyclyl rings in which the bridged structure is embedded.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share one common bond with, one or more heterocyclyl or carbocyclyl groups, as defined above, wherein the point of attachment is on any of the fused rings. In such instances, the number of carbons designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than one atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the bridged rings.

“Hetero” when used to describe a compound or a group present on a compound means that one or more carbon atoms in the compound or group have been replaced by a nitrogen, oxygen, sulfur, boron, phosphorus, and silicon heteroatom, as valency permits. Hetero may be applied to any of the hydrocarbyl groups described above having from 1 to 5, and particularly from 1 to 3 heteroatoms.

“Acyl” as used herein, refers to a radical —C(O)R, wherein R is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative acyl groups include, but are not limited to, formyl (—CHO), acetyl (—C(═O)CH₃), cyclohexylcarbonyl, cyclohexylmethylcarbonyl, benzoyl (—C(═O)Ph), and benzylcarbonyl (—C(═O)CH₂Ph).

“Acylamino” as used herein, refers to a radical —NRC(═O)R, wherein each instance of R is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Exemplary “acylamino” groups include, but are not limited to, formylamino, acetylamino, cyclohexylcarbonylamino, cyclohexylmethyl-carbonylamino, benzoylamino and benzylcarbonylamino.

“Acyloxy” as used herein, refers to a radical —OC(═O)R, wherein R is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative examples include, but are not limited to, formyl, acetyl, cyclohexylcarbonyl, cyclohexylmethylcarbonyl, benzoyl and benzylcarbonyl.

“Alkoxy” as used herein, refers to the group —OR, wherein R is alkyl as defined herein. C₁₋₆ alkoxy refers to the group —OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Azido” refers to the radical —N₃.

“Amino” refers to the radical —NH₂.

“Hydroxy” refers to the radical —OH.

“Thioketo” refers to the group ═S.

“Carboxy” refers to the radical —C(═O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Nitro” refers to the radical —NO₂.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl(Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention.

The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof).

In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both.

In a further embodiment, “treating” or “treatment” relates to slowing the progression of the disease.

It is also to be understood that compounds that have the same molecular formula but differ in the nature or sequence of bonding of their atoms or the arrangement of their atoms in space are termed “isomers.” Isomers that only differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R - and S -sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In certain embodiments, the weights are based upon total weight of compound.

As used herein and unless otherwise indicated, the term “enantiomerically pure (S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### Examples

In order that the invention described herein may be more fully understood, the following examples are set forth. The examples described in this application are offered to illustrate the compounds, pharmaceutical compositions, and methods provided herein and are not to be construed in any way as limiting their scope.

### I. Synthesis and Characterization

### Compound 1. (R)-3-((4-((1R,6R)-7,7-difluoro-3-azabicyclo[4.1.0]heptan-6-yl)phenyl)amino)piperidine-2,6-dione

### Compound 2. (S)-3-((4-((1R,6R)-7,7-difluoro-3-azabicyclo[4.1.0]heptan-6-yl)phenyl)amino)piperidine-2,6-dione

### Compound 3. (R)-3-((4-((1S,6S)-7,7-difluoro-3-azabicyclo[4.1.0]heptan-6-yl)phenyl)amino)piperidine-2,6-dione

### Compound 4. (S)-3-((4-((1S,6S)-7,7-difluoro-3-azabicyclo[4.1.0]heptan-6-yl)phenyl)amino)piperidine-2,6-dione

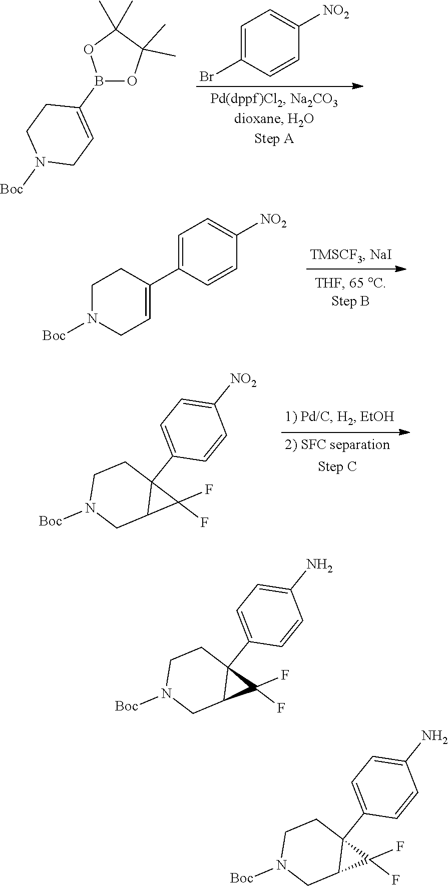

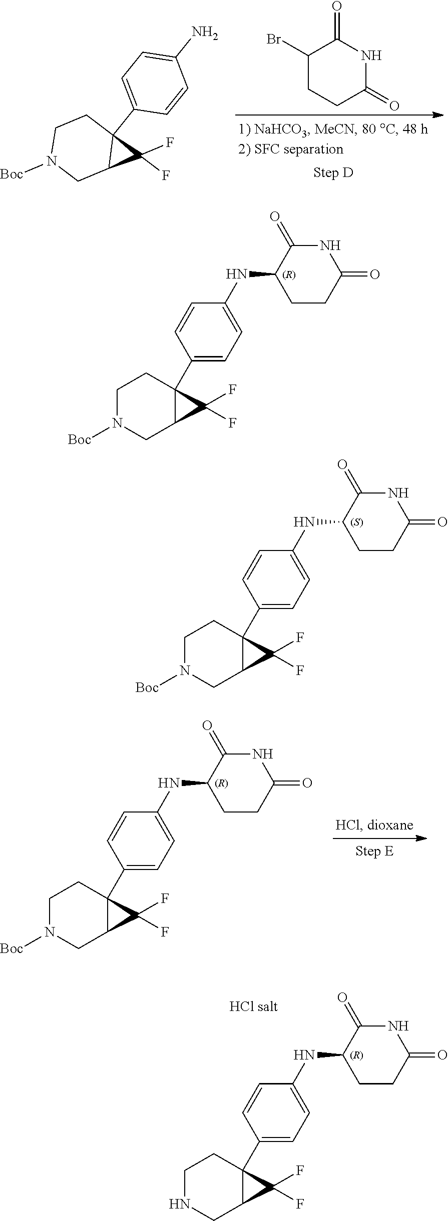

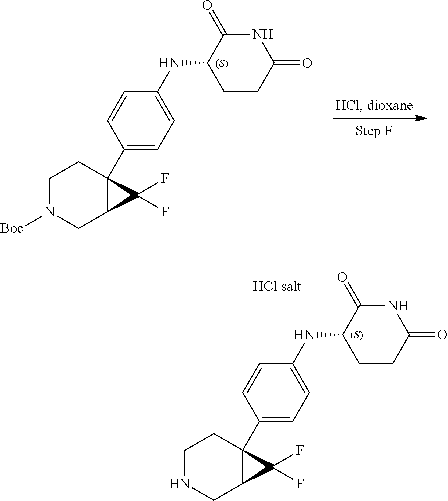

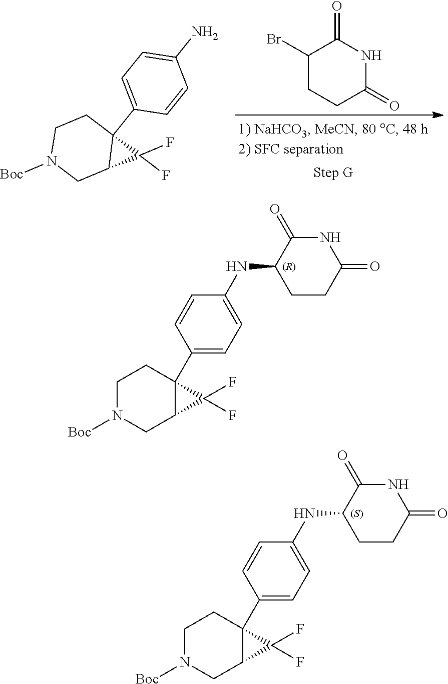

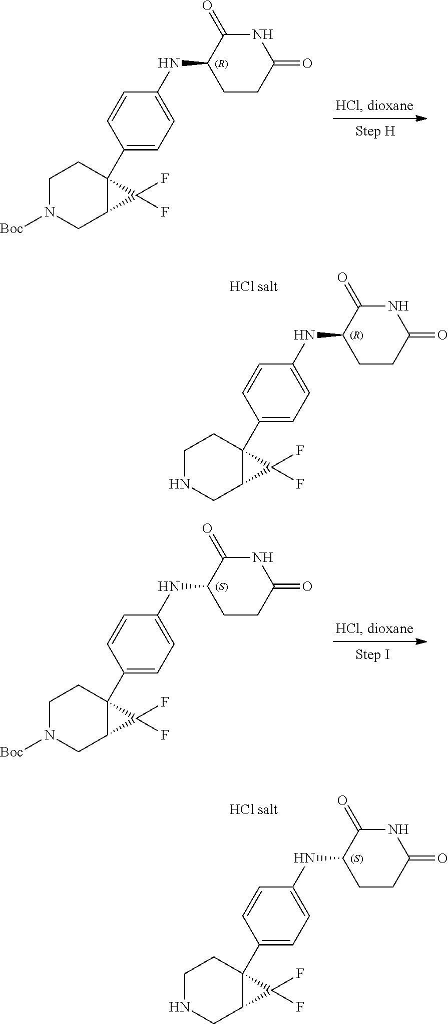

### Step A: tert-butyl 4-(4-nitrophenyl)-3,6-dihydropyridine-1(2H)-carboxylate

A mixture of tert-butyl 4-(4,4,5,5-tetramethyl-1,3,2-dioxaborolan-2-yl)-3,6-dihydropyridine-1(2H)-carboxylate (9.51 g, 1.1 eq, 30.8 mmol), 1-bromo-4-nitrobenzene (5.65 g, 1 eq, 28.0 mmol), Pd(dppf)Cl₂ (2.05 g, 0.1 eq, 2.8 mmol) and Na₂CO₃ (8.89 g, 3 eq, 83.9 mmol) in 1,4-Dioxane (100 mL) and H₂O (20 mL) was stirred at 100° C. under N₂ atmosphere for 3 h. The mixture was diluted with water (100 mL) and extracted with EA (100 mL×3).

The combined organic layers were washed with brine (100 mL), dried over anhydrous Na₂SO₄ and concentrated under reduced pressure. The residue was purified by flash chromatography and eluted with 0-30% EA in PE to give tert-butyl 4-(4-nitrophenyl)-3,6-dihydropyridine-1(2H)-carboxylate (7.2 g, yield 86%) as a yellow solid. LC-MS (ESI): mass calcd. for C₁₆H₂₀N₂O₄, 304.14; m/z found, 305 [M+H]⁺.

### Step B: tert-butyl 7,7-d fluoro-6-(4-nitrophenyl)-3-azabicyclo[4.1.0]heptane-3-carboxylate

To a solution of tert-butyl 4-(4-nitrophenyl)-3,6-dihydropyridine-1(2H)-carboxylate (6 g, 1 eq, 19.7 mmol) and sodium iodide (2.95 g, 1 eq, 19.7 mmol) in THF (60 mL) was added trifluoromethyltrimethylsilane (29.5 mL, 10 eq, 197 mmol) at room temperature. The mixture was stirred at 65° C. overnight under N₂ atmosphere. Then the reaction mixture was concentrated under reduced pressure. The residue was purified by flash chromatography and eluted with 0-30% EA in PE to afford tert-butyl 7,7-difluoro-6-(4-nitrophenyl)-3-azabicyclo[4.1.0]heptane-3-carboxylate (6 g, yield 86%) as a pale yellow solid. LC-MS (ESI): mass calcd. for C₁₇H₂₀F₂N₂04, 354.14; m/z found, 340 [M+H-56+MeCN]⁺.

### Step C: tert-butyl (1R,6R)-6-(4-aminophenyl)-7,7-difluoro-3-azabicyclo[4.1.0]heptane-3-carboxylate & tert-butyl (1S,6S)-6-(4-aminophenyl)-7,7-difluoro-3-azabicyclo[4.1.0]heptane-3-carboxylate

A mixture of tert-butyl 7,7-difluoro-6-(4-nitrophenyl)-3-azabicyclo[4.1.0]heptane-3-carboxylate (5 g, 1 eq, 14.1 mmol) and 10% Pd—C(0.50 g) in EtOH (50 mL) was stirred at room temperature under hydrogen atmosphere for 2 h. The mixture was filtered through a Celite pad and the filtrate was concentrated under reduced pressure. The residue was purified by flash chromatography and eluted with 0-40% EA in DCM and SFC separation to afford tert-butyl (1R,6R)-6-(4-aminophenyl)-7,7-difluoro-3-azabicyclo[4.1.0]heptane-3-carboxylate (1.3 g, yield 28%) as a yellow oil, LC-MS (ESI): mass calcd. for C₁₇H₂₂F₂N₂O₂, 324.16; m/z found, 325 [M+H]⁺; and tert-butyl (1S,6S)-6-(4-aminophenyl)-7,7-difluoro-3-azabicyclo[4.1.0]heptane-3-carboxylate (1.3 g, yield 28%) as a yellow oil, LC-MS (ESI): mass calcd. for C₁₇H₂₂F₂N₂O₂, 324.16; m/z found, 325 [M+H]⁺.

### Preparative Separation Method

Instrument: SHIMADZU PReP SOLUTION SFC
Column: ChiralPak IC, 250×21.2 mm I.D., 5 μm
Mobile phase: A for C₀₂ and B for MeOH+0.1% NH₃H₂O
Gradient: B 40%
Flow rate: 40 mL/min
Back pressure: 100 bar
Column temperature: 35° C.
Wavelength: 220 nm
Cycle-time: 5 min
eluted time: 3 h

### Step D: tert-butyl (1R,6R)-6-(4-(((R)-2,6-dioxopiperidin-3-yl)amino)phenyl)-7,7-difluoro-3-azabicyclo[4.1.0]heptane-3-carboxylate & tert-butyl (1R,6R)-6-(4-(((S)-2,6-dioxopiperidin-3-yl)amino)phenyl)-7,7-difluoro-3-azabicyclo[4.1.0]heptane-3-carboxylate

A mixture of tert-butyl (1R,6R)-6-(4-aminophenyl)-7,7-difluoro-3-azabicyclo[4.1.0]heptane-3-carboxylate (1.3 g, 1 eq, 4.01 mmol), 3-bromopiperidine-2,6-dione (923 mg, 1.2 eq, 4.81 mmol) and sodium bicarbonate (1.01 g, 3 eq, 12 mmol) in MeCN (15 mL) was stirred at 80° C. for 48 h. The mixture was diluted with water (10 mL) and extracted with EA (10 mL×3). The combined organic layers were washed with brine (20 mL), dried over anhydrous Na₂SO₄ and concentrated under reduced pressure. The residue was purified by flash chromatography and eluted with 0-40% EA in DCM and SFC separation to give tert-butyl (1R,6R)-6-(4-(((R)-2,6-dioxopiperidin-3-yl)amino)phenyl)-7,7-difluoro-3-azabicyclo[4.1.0]heptane-3-carboxylate (440 mg, yield 26%) as a blue solid, LC-MS (ESI): mass calcd. for C₂₂H₂₇F₂N₃O₄, 435.20; m/z found, 436 [M+H]⁺; and tert-butyl (1R,6R)-6-(4-(((S)-2,6-dioxopiperidin-3-yl)amino)phenyl)-7,7-difluoro-3-azabicyclo[4.1.0]heptane-3-carboxylate (480 mg, yield 31%) as a blue solid, LC-MS (ESI): mass calcd. for C₂₂H₂₇F₂N₃O₄, 435.20; m/z found, 436 [M+H]⁺.

### Preparative Separation Method

Instrument: Waters Thar 80 preparative SFC
Column: ChiralPak IH, 250×21.2 mm I.D., 5 μm
Mobile phase: A for C₀₂ and B for MeOH+0.1% NH₃H₂O
Gradient: B 20%
Flow rate: 40 mL/min
Back pressure: 100 bar
Column temperature: 35° C.
Wavelength: 254 nmCycle-time: 3 min
eluted time: 6H

### Step E: (R)-3-((4-((1R,6R)-7,7-difluoro-3-azabicyclo[4.1.0]heptan-6-yl)phenyl)amino)piperidine-2,6-dione

A mixture of tert-butyl (1R,6R)-6-(4-(((R)-2,6-dioxopiperidin-3-yl)amino)phenyl)-7,7-difluoro-3-azabicyclo[4.1.0]heptane-3-carboxylate (440 mg, 1 eq, 1.01 mmol) and HCl (4 M in dioxane; 1.3 mL, 5.2 eq, 5.2 mmol) in DCM (5 mL) was stirred at room temperature for 16 h. The reaction was monitored by LCMS. Then the precipitate was collected by filtration to afford (R)-3-((4-((1R,6R)-7,7-difluoro-3-azabicyclo[4.1.0]heptan-6-yl)phenyl)amino)piperidine-2,6-dione HCl salt (348.4 mg, yield 93%) as a blue solid. LC-MS (ESI): mass calcd. for C₁₇H₁₉F₂N₃O₂, 335.14; m/z found, 336 [M+H]⁺.

¹HNMR (400 MHz, MeOD) δ 7.66 (d, J=8.6 Hz, 2H), 7.46 (d, J=8.6 Hz, 2H), 4.77-4.72 (m, 1H), 3.89-3.80 (m, 1H), 3.37-3.35 (m, 1H), 3.33-3.25 (m, 1H), 3.00-2.94 (m, 1H), 2.77-2.68 (m, 2H), 2.59-2.45 (m, 2H), 2.28-2.19 (m, 1H), 2.13-2.03 (m, 2H).

¹⁹FNMR (376 MHz, MeOD) δ −131.77 (d, J=157.7 Hz), −144.99 (d, J=158.0 Hz).

### Step F: (S)-3-((4-((1R,6R)-7,7-difluoro-3-azabicyclo[4.1.0]heptan-6-yl)phenyl)amino)piperidine-2,6-dione

A mixture of tert-butyl (1R,6R)-6-(4-(((S)-2,6-dioxopiperidin-3-yl)amino)phenyl)-7,7-difluoro-3-azabicyclo[4.1.0]heptane-3-carboxylate (480 mg, 1 eq, 1.1 mmol) and HCl (4 M in dioxane; 1.4 mL, 5.1 eq, 5.6 mmol) in DCM (5 mL) was stirred at room temperature for 16 h. The reaction was monitored by LCMS. Then the precipitate was collected by filtration to afford (S)-3-((4-((1R,6R)-7,7-difluoro-3-azabicyclo[4.1.0]heptan-6-yl)phenyl)amino)piperidine-2,6-dione HCl salt (376.8 mg, yield 92%) as a blue solid. LC-MS (ESI): mass calcd. for C₁₇H₁₉F₂N₃O₂, 335.14; m/z found, 336 [M+H]⁺.

¹HNMR (400 MHz, MeOD) δ 7.72 (d, J=8.6 Hz, 2H), 7.56 (d, J=8.6 Hz, 2H), 4.94-4.78 (m, 1H), 3.94-3.89 (m, 1H), 3.37-3.35 (m, 1H), 3.33-3.25 (m, 1H), 3.02-2.94 (m, 1H), 2.77-2.68 (m, 2H), 2.59-2.45 (m, 2H), 2.28-2.19 (m, 1H), 2.13-2.03 (m, 2H).

¹⁹FNMR (376 MHz, MeOD) δ −131.87 (d, J=157.7 Hz), −144.78 (d, J=158.0 Hz).

### Step G: tert-butyl (1S,6S)-6-(4-(((R)-2,6-dioxopiperidin-3-yl)amino)phenyl)-7,7-difluoro-3-azabicyclo[4.1.0]heptane-3-carboxylate & tert-butyl (1S,6S)-6-(4-(((S)-2,6-dioxopiperidin-3-yl)amino)phenyl)-7,7-difluoro-3-azabicyclo[4.1.0]heptane-3-carboxylate

A mixture of tert-butyl (1S,6S)-6-(4-aminophenyl)-7,7-difluoro-3-azabicyclo[4.1.0]heptane-3-carboxylate (1.3 g, 1 eq, 4.01 mmol), 3-bromopiperidine-2,6-dione (923 mg, 1.2 eq, 4.81 mmol) and sodium bicarbonate (1.01 g, 3 eq, 12 mmol) in MeCN (15 mL) was stirred at 80° C. for 48 h. The mixture was diluted with water (10 mL) and extracted with EA (10 mL×3). The combined organic layers were washed with brine (20 mL), dried over anhydrous Na₂SO₄ and concentrated under reduced pressure. The residue was purified by flash chromatography and eluted with 0-40% EA in DCM and SFC separation to give tert-butyl (1S,6S)-6-(4-(((R)-2,6-dioxopiperidin-3-yl)amino)phenyl)-7,7-difluoro-3-azabicyclo[4.1.0]heptane-3-carboxylate (400 mg, yield 23%) as a blue solid, LC-MS (ESI): mass calcd. for C₂₂H27F₂N₃O₄, 435.20; m/z found, 436 [M+H]⁺; and tert-butyl (1S,6S)-6-(4-(((S)-2,6-dioxopiperidin-3-yl)amino)phenyl)-7,7-difluoro-3-azabicyclo[4.1.0]heptane-3-carboxylate (400 mg, yield 23%) as a blue solid, LC-MS (ESI): mass calcd. for C₂₂H27F₂N₃O₄, 435.20; m/z found, 436 [M+H]⁺.

### Preparative Separation Method

Instrument: Waters Thar 80 preparative SFC
Column: ChiralPak IH, 250×21.2 mm I.D., 5 μm
Mobile phase: A for C₀₂ and B for MeOH+0.1% NH₃H₂O
Gradient: B 20%
Flow rate: 40 mL/min
Back pressure: 100 bar
Column temperature: 35° C.
Wavelength: 254 nm
Cycle-time: 3 min
eluted time: 6H

### Step H: (R)-3-((4-((1S,6S)-7,7-difluoro-3-azabicyclo[4.1.0]heptan-6-yl)phenyl)amino)piperidine-2,6-dione

A mixture of tert-butyl (1S,6S)-6-(4-(((R)-2,6-dioxopiperidin-3-yl)amino)phenyl)-7,7-difluoro-3-azabicyclo[4.1.0]heptane-3-carboxylate (400 mg, 1 eq, 0.92 mmol) and HCl (4 M in dioxane; 1.2 mL, 5.2 eq, 4.8 mmol) in DCM (5 mL) was stirred at room temperature for 16 h. The reaction was monitored by LCMS. Then the precipitate was collected by filtration to afford (R)-3-((4-((1S,6S)-7,7-difluoro-3-azabicyclo[4.1.0]heptan-6-yl)phenyl)amino)piperidine-2,6-dione HCl salt (300 mg, yield 97%) as a blue solid. LC-MS (ESI): mass calcd. for C₁₇H₁₉F₂N₃O₂, 335.14; m/z found, 336 [M+H]⁺.

¹HNMR (400 MHz, MeOD) δ 7.68 (d, J=8.5 Hz, 2H), 7.48 (d, J=8.7 Hz, 2H), 4.81-4.71 (m, 1H), 3.93-3.87 (m, 1H), 3.37-3.35 (m, 1H), 3.33-3.25 (m, 1H), 3.04-2.91 (m, 1H), 2.77-2.68 (m, 2H), 2.61-2.45 (m, 2H), 2.28-2.19 (m, 1H), 2.15-2.01 (m, 2H).

¹⁹FNMR (376 MHz, MeOD) δ −131.95 (d, J=157.9 Hz), −144.78 (d, J=157.8 Hz).

### Step I: (S)-3-((4-((1S,6S)-7,7-difluoro-3-azabicyclo[4.1.0]heptan-6-yl)phenyl)amino)piperidine-2,6-dione

A mixture of tert-butyl (1S,6S)-6-(4-(((S)-2,6-dioxopiperidin-3-yl)amino)phenyl)-7,7-difluoro-3-azabicyclo[4.1.0]heptane-3-carboxylate (400 mg, 1 eq, 0.92 mmol) and HCl (4 M in dioxane; 1.2 mL, 5.2 eq, 4.8 mmol) in DCM (5 mL) was stirred at room temperature for 16 h. The reaction was monitored by LCMS. Then the precipitate was collected by filtration to afford (S)-3-((4-((1S,6S)-7,7-difluoro-3-azabicyclo[4.1.0]heptan-6-yl)phenyl)amino)piperidine-2,6-dione HCl salt (300 mg, yield 97%) as a blue solid. LC-MS (ESI): mass calcd. for C₁₇H₁₉F₂N₃O₂, 335.14; m/z found, 336 [M+H]⁺.

¹HNMR (400 MHz, MeOD) δ 7.62 (d, J=8.5 Hz, 2H), 7.40 (d, J=8.7 Hz, 2H), 4.72-4.68 (m, 1H), 3.92-3.86 (m, 1H), 3.37-3.35 (m, 1H), 3.33-3.25 (m, 1H), 3.03-2.91 (m, 1H), 2.77-2.68 (m, 2H), 2.61-2.45 (m, 2H), 2.28-2.19 (m, 1H), 2.15-2.00 (m, 2H).

¹⁹FNMR (376 MHz, MeOD) δ −132.07 (d, J=157.9 Hz), −144.77 (d, J=157.8 Hz).

### Compound 5. (R)-3-((2H-spiro[benzofuran-3,4′-piperidin]-6-yl)oxy)piperidine-2,6-dione

### Compound 6. (S)-3-((2H-spiro[benzofuran-3,4′-piperidin]-6-yl)oxy)piperidine-2,6-dione

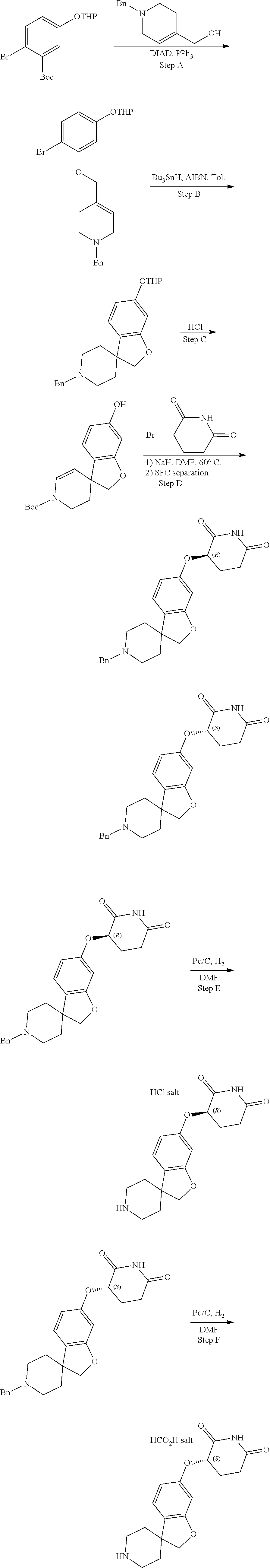

### Step A: 1-benzyl-4-((2-bromo-5-((tetrahydro-2H-pyran-2-yl)oxy)phenoxy)methyl)-1,2,3,6-tetrahydropyridine

To a solution of 2-bromo-5-((tetrahydro-2H-pyran-2-yl)oxy)phenol (3.3 g, 1 eq, 12.1 mmol; Ref Chemical Communications 2009, 19, 2756-2758) and (1-benzyl-1,2,3,6-tetrahydropyridin-4-yl)methanol (2.46 g, 1 eq, 12.1 mmol) in THF (33 mL) was added triphenylphosphane (4.75 g, 1.5 eq, 18.1 mmol). The mixture was cooled to 0° C. under N₂ atmosphere. DIAD (4.03 g, 1.65 eq, 19.9 mmol) was added dropwise and then the mixture was stirred at room temperature overnight. The reaction mixture was concentrated to give a residue, which was purified by flash chromatography and eluted with 0-20% EA in PE to give 5.5 g of crude product. 100 ML of n-heptane was added to the crude product and the mixture was heated until a clear solution formed. The hot solution was left to reach room temperature and filtered. The filtrate was concentrated to give 1-benzyl-4-((2-bromo-5-((tetrahydro-2H-pyran-2-yl)oxy)phenoxy)methyl)-1,2,3,6-tetrahydropyridine (3.53 g, yield 64%) as a light yellow oil. LC-MS (ESI): mass calcd. for C₂₄H₂₈BrNO₃, 457.13/459.13; m/z found, 458/460 [M+H]⁺.

### Step B: 1′-benzyl-6-((tetrahydro-2H-pyran-2-yl)oxy)-2H-spiro[benzofuran-3,4′-piperidine]

To a solution of 1-benzyl-4-((2-bromo-5-((tetrahydro-2H-pyran-2-yl)oxy)phenoxy)methyl)-1,2,3,6-tetrahydropyridine (3.53 g, 1 eq, 7.7 mmol) in toluene (60 mL) was added tributylstannane (6.2 mL, 3 eq, 23.1 mmol) and then AIBN (253 mg, 0.2 eq, 1.54 mmol). The mixture was stirred at 110° C. overnight. The mixture was quenched with sat. KF aq. (50 mL) and stirred for at least 0.5 h., extracted with EA (60 mL×3). The combined organic layers were washed with brine (60 mL), dried over anhydrous Na₂SO₄ and concentrated under reduced pressure. The residue was purified by flash chromatography and eluted with 20-50% EA in PE to give the 1′-benzyl-6-((tetrahydro-2H-pyran-2-yl)oxy)-2H-spiro[benzofuran-3,4′-piperidine](2.85 g, yield 98%) as a light yellow oil. LC-MS (ESI): mass calcd. for C₂₄H₂₉NO₃, 379.21; m/z found, 380 [M+H]⁺.

### Step C: 1′-benzyl-2H-spiro[benzofuran-3,4′-piperidin]-6-ol

To the solution of 1′-benzyl-6-((tetrahydro-2H-pyran-2-yl)oxy)-2H-spiro[benzofuran-3,4′-piperidine](2.83 g, 1 eq, 7.46 mmol) in MeOH (30 mL) was added 8 mL of 4N HCl/dioxane. The mixture was stirred at 50° C. for 5 h. The mixture was concentrated to give a residue, which was purified by flash chromatography and eluted with 30-100% EA in PE to give 1′-benzyl-2H-spiro[benzofuran-3,4′-piperidin]-6-ol (1.5 g, yield 68%) as a white solid. LC-MS (ESI): mass calcd. for C₁₉H₂₁NO₂, 295.16; m/z found, 296 [M+H]⁺.

### Step D: (R)-3-((1′-benzyl-2H-spiro[benzofuran-3,4′-piperidin]-6-yl)oxy)piperidine-2,6-dione & (S)-3-((1′-benzyl-2H-spiro[benzofuran-3,4′-piperidin]-6-yl)oxy)piperidine-2,6-dione

To a solution of 1′-benzyl-2H-spiro[benzofuran-3,4′-piperidin]-6-ol (350 mg, 1 eq, 1.18 mmol) in THF (5 mL) was added sodium hydride (60% in mineral oil; 57 mg, 1.2 eq, 1.42 mmol) at 60° C. After 20 min, 3-bromopiperidine-2,6-dione (273 mg, 1.2 eq, 1.42 mmol) was added to above solution. Then the mixture was stirred at the same temperature for 1 h. The reaction was quenched with sat. NH₄Cl aq. (10 mL), diluted with water (20 mL) and extracted with EA (30 mL×3). The combined organic layers were washed with brine (30 mL), dried over anhydrous Na₂SO₄ and concentrated under reduced pressure. The residue was purified by Prep-TLC (DCM/MeOH=12/1) and separated by SFC to afford (R)-3-((1′-benzyl-2H-spiro[benzofuran-3,4′-piperidin]-6-yl)oxy)piperidine-2,6-dione (80 mg, yield 17%) as a white solid, LC-MS (ESI): mass calcd. for C₂₄H₂₆N₂O₄, 406.19; m/z found, 407 [M+H]⁺; and (S)-3-((1′-benzyl-2H-spiro[benzofuran-3,4′-piperidin]-6-yl)oxy)piperidine-2,6-dione (80 mg, yield 17%) as a white solid. LC-MS (ESI): mass calcd. for C₂₄H₂₆N₂O₄, 406.19; m/z found, 407 [M+H]⁺.

### Preparative Separation Method

Instrument: SHIMADZU PREP SOLUTION SFC
Column: ChiralCel OJ, 250×21.2 mm I.D., 5 μm
Mobile phase: A for CO₂ and B for MeOH
Gradient: B 28%
Flow rate: 40 mL/min
Back pressure: 100 bar
Column temperature: 35° C.
Wavelength: 210 nm
Cycle-time: 5.5 min
Eluted time: 4 h

### Step E: (R)-3-((2H-spiro[benzofuran-3,4′-piperidin]-6-yl)oxy)piperidine-2,6-dione

A mixture of (R)-3-((1′-benzyl-2H-spiro[benzofuran-3,4′-piperidin]-6-yl)oxy)piperidine-2,6-dione (80 mg, 1 eq, 0.197 mmol), 10% Pd/C (16 mg) in DMF (5 mL) was stirred at room temperature under H₂ atmosphere for 48 h. The reaction was monitored by LCMS. The mixture was filtered through a Celite pad and the filtrate was concentrated under reduced pressure. The residue was purified by Prep-HPLC (C18, 0.1% HCl) to afford (R)-3-((2H-spiro[benzofuran-3,4′-piperidin]-6-yl)oxy)piperidine-2,6-dione HCl salt (23 mg, yield 33%) as a white solid. LC-MS (ESI): mass calcd. for C₁₇H₂₀N₂O₄, 316.14; m/z found, 317 [M+H]⁺.

¹H NMR (400 MHz, MeOD-d₄) δ 7.11 (d, J=8.3 Hz, 1H), 6.61 (dd, J=8.3, 2.2 Hz, 1H), 6.55 (d, J=2.2 Hz, 1H), 5.04 (dd, J=9.9, 4.8 Hz, 1H), 4.49 (s, 2H), 3.47-3.38 (m, 2H), 3.21-3.11 (m, 2H), 2.78-2.70 (m, 2H), 2.33-2.24 (m, 1H), 2.23-2.15 (m, 1H), 2.13-2.04 (m, 2H), 2.01-1.94 (m, 2H).

### Step F: (S)-3-((2H-spiro[benzofuran-3,4′-piperidin]-6-yl)oxy)piperidine-2,6-dione

A mixture of (S)-3-((1′-benzyl-2H-spiro[benzofuran-3,4′-piperidin]-6-yl)oxy)piperidine-2,6-dione (80 mg, 1 eq, 0.197 mmol), 10% Pd/C (16 mg) in DMF (5 mL) was stirred at room temperature under H₂ atmosphere for 48 h. The reaction was monitored by LCMS. The mixture was filtered through a Celite pad and the filtrate was concentrated under reduced pressure. The residue was purified by Prep-HPLC (C18, 0.1% FA) afford (S)-3-((2H-spiro[benzofuran-3,4′-piperidin]-6-yl)oxy)piperidine-2,6-dione FA salt (8 mg, yield 11%) as a white solid. LC-MS (ESI): mass calcd. for C₁₇H₂₀N₂O₄, 316.14; m/z found, 317 [M+H]⁺.

¹H NMR (400 MHz, MeOD-d₄, HCO₂H salt) δ 8.53 (s, 1H), 7.10 (d, J=8.3 Hz, 1H), 6.61 (dd, J=8.3, 2.2 Hz, 1H), 6.55 (d, J=2.2 Hz, 1H), 5.04 (dd, J=9.9, 4.8 Hz, 1H), 4.49 (s, 2H), 3.45-3.38 (m, 2H), 3.19-3.07 (m, 2H), 2.78-2.71 (m, 2H), 2.34-2.26 (m, 1H), 2.23-2.14 (m, 1H), 2.10-2.02 (m, 2H), 1.99-1.87 (m, 2H).

### Compound 7. (R)-3-((4-((1R,6R)-7,7-difluoro-3-azabicyclo[4.1.0]heptan-6-yl)-2-methylphenyl)amino)piperidine-2,6-dione

### Compound 8. (S)-3-((4-((1R,6R)-7,7-difluoro-3-azabicyclo[4.1.0]heptan-6-yl)-2-methylphenyl)amino)piperidine-2,6-dione

### Compound 9. (R)-3-((4-((1S,6S)-7,7-difluoro-3-azabicyclo[4.1.0]heptan-6-yl)-2-methylphenyl)amino)piperidine-2,6-dione

### Compound 10. (S)-3-((4-((1S,6S)-7,7-difluoro-3-azabicyclo[4.1.0]heptan-6-yl)-2-methylphenyl)amino)piperidine-2,6-dione

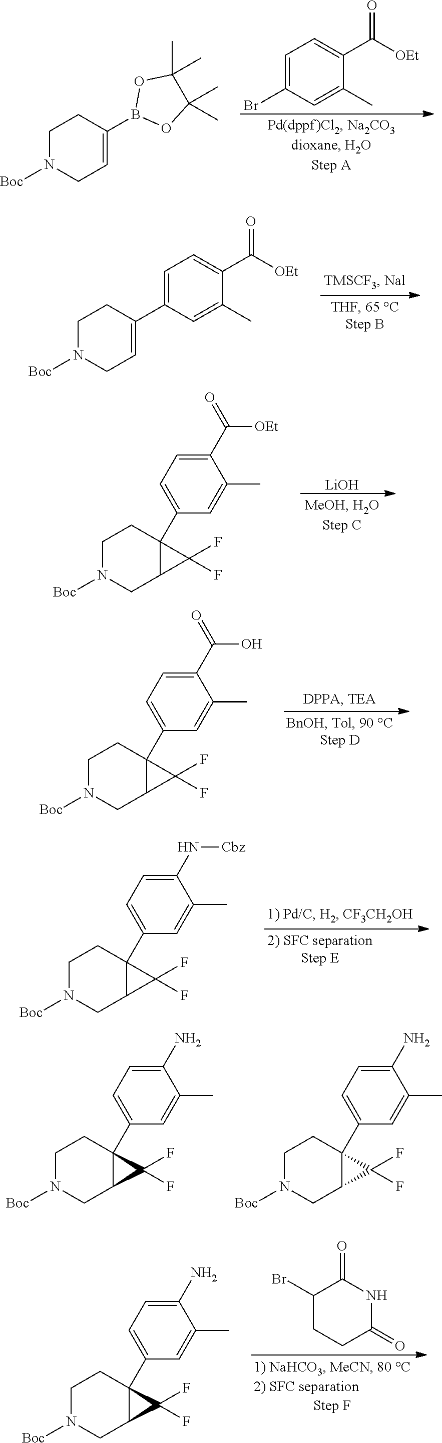

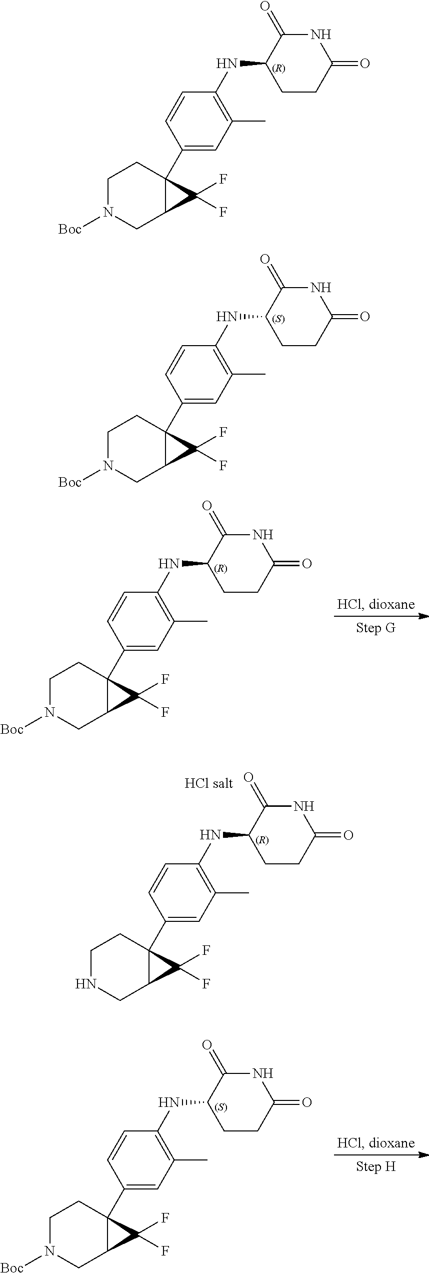

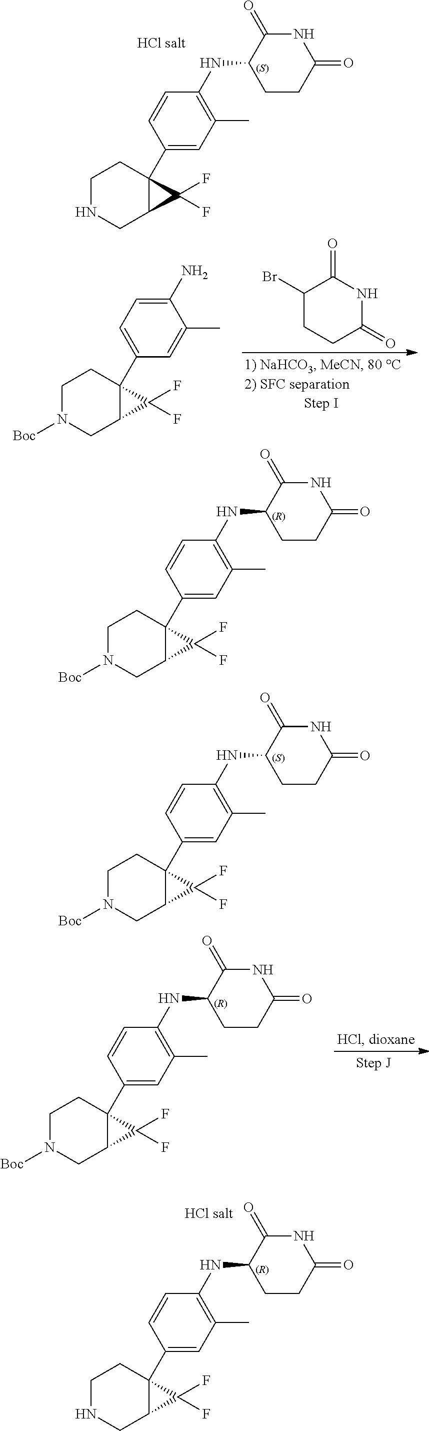

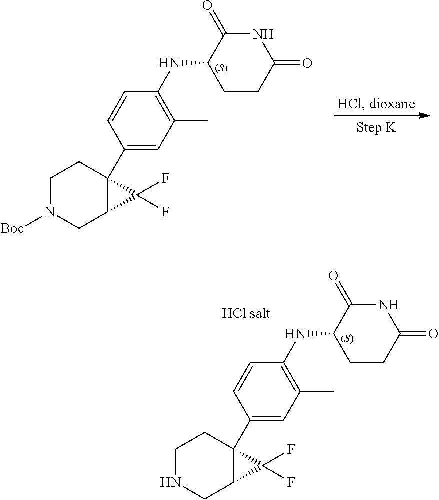

### Step A: tert-butyl 4-(4-(ethoxycarbonyl)-3-methylphenyl)-3,6-dihydropyridine-1(2H)-carboxylate

A mixture of tert-butyl 4-(4,4,5,5-tetramethyl-1,3,2-dioxaborolan-2-yl)-3,6-dihydropyridine-1(2H)-carboxylate (3.4 g, 1.1 eq, 11 mmol), ethyl 4-bromo-2-methylbenzoate (2.42 g, 1 eq, 10 mmol), Pd(dppf)Cl₂ (0.73 g, 0.1 eq, 1 mmol) and Na₂CO₃ (3.18 g, 3 eq, 30 mmol) in 1,4-Dioxane (20 mL) and H₂O (5 mL) was stirred at 100° C. under N₂ atmosphere for 3 h. The mixture was diluted with water (20 mL) and extracted with EA (20 mL×3). The combined organic layers were washed with brine (20 mL), dried over anhydrous Na₂SO₄ and concentrated under reduced pressure. The residue was purified by flash chromatography and eluted with 0-30% EA in PE to give tert-butyl 4-(4-(ethoxycarbonyl)-3-methylphenyl)-3,6-dihydropyridine-1(2H)-carboxylate (2.7 g, yield 78%) as a yellow solid. LC-MS (ESI): mass calcd. for C₂₀H₂₇NO₄, 345.19; m/z found, 346 [M+H]⁺.

### Step B: tert-butyl 6-(4-(ethoxycarbonyl)-3-methylphenyl)-7,7-difluoro-3-azabicyclo[4.1.0]heptane-3-carboxylate

To a solution of tert-butyl 4-(4-(ethoxycarbonyl)-3-methylphenyl)-3,6-dihydropyridine-1(2H)-carboxylate (5 g, 1 eq, 14.5 mmol) and Sodium iodide (2.17 g, 1 eq, 14.5 mmol) in THF (60 mL) was added trifluoromethyltrimethylsilane (21.7 mL, 10 eq, 145 mmol) at room temperature. The mixture was stirred at 65° C. overnight under N₂ atmosphere. Then the reaction mixture was concentrated under reduced pressure. The residue was purified by flash chromatography and eluted with 0-30% EA in PE to afford tert-butyl 6-(4-(ethoxycarbonyl)-3-methylphenyl)-7,7-difluoro-3-azabicyclo[4.1.0]heptane-3-carboxylate (4 g, yield 70%) as a pale yellow solid. LC-MS (ESI): mass calcd. for C₂₁H₂₇F₂NO₄, 395.19; m/z found, 340 [M+H-56]⁺.

### Step C: 4-(3-(tert-butoxycarbonyl)-7,7-difluoro-3-azabicyclo[4.1.0]heptan-6-yl)-2-methylbenzoic acid

A mixture of tert-butyl 6-(4-(ethoxycarbonyl)-3-methylphenyl)-7,7-difluoro-3-azabicyclo[4.1.0]heptane-3-carboxylate (2 g, 1 eq, 5.06 mmol), LiOH (606 mg, 5 eq, 25.3 mmol), THF (9 mL), MeOH (9 mL) and H₂O (3 mL) was stirred at room temperature overnight. The reaction mixture was adjusted to pH 4-5 with 1M HCl aq. Then the mixture was extracted with EA (20 mL×3) and washed with brine (20 mL). The combined organic layers were dried over anhydrous Na₂SO₄ and concentrated under reduced pressure. The residue was used in the next step. LC-MS (ESI): mass calcd. for C₁₉H₂₃F₂NO₄, 367.16; m/z found, 366 [M−H]—.

### Step D: tert-butyl 6-(4-(((benzyloxy)carbonyl)amino)-3-methylphenyl)-7,7-difluoro-3-azabicyclo[4.1.0]heptane-3-carboxylate

A solution of 4-(3-(tert-butoxycarbonyl)-7,7-difluoro-3-azabicyclo[4.1.0]heptan-6-yl)-2-methylbenzoic acid (600 mg, 1 eq, 1.63 mmol) and diphenylphosphoryl azide (539 mg, 1.2 eq, 1.96 mmol) in toluene (10 mL) was stirred at 80° C. for 15 min. Then triethylamine (0.7 mL, 3 eq, 4.9 mmol) was added to the above solution. The mixture was stirred at 80° C. for 2 h under N₂ atmosphere. Benzyl alcohol (0.5 mL, 3 eq, 4.9 mmol) was added and the mixture was stirred at the same temperature overnight. The mixture was diluted with water (10 mL) and extracted with EA (10 mL×3). The combined organic layers were washed with brine (10 mL), dried over anhydrous Na₂SO₄ and concentrated under reduced pressure. The residue was purified by flash chromatography and eluted with 0-50% EA in PE to afford tert-butyl 6-(4-(((benzyloxy)carbonyl)amino)-3-methylphenyl)-7,7-difluoro-3-azabicyclo[4.1.0]heptane-3-carboxylate (500 mg, yield 65%) as a white solid. LC-MS (ESI): mass calcd. for C₂₆H₃₀F₂N₂04, 472.22; m/z found, 473 [M+H]⁺.

### Step E: tert-butyl (1R,6R)-6-(4-amino-3-methylphenyl)-7,7-difluoro-3-azabicyclo[4.1.0]heptane-3-carboxylate & tert-butyl (1S,6S)-6-(4-amino-3-methylphenyl)-7,7-difluoro-3-azabicyclo[4.1.0]heptane-3-carboxylate

A mixture of tert-butyl 6-(4-(((benzyloxy)carbonyl)amino)-3-methylphenyl)-7,7-difluoro-3-azabicyclo[4.1.0]heptane-3-carboxylate (600 mg, 1.27 mmol) and 10% Pd/C (120 mg) in TFE (10 mL) was stirred at room temperature under hydrogen atmosphere for 2 h. The mixture was filtered through a Celite pad and the filtrate was concentrated under reduced pressure. The residue was purified by flash chromatography and eluted with 0-70% EA in PE to and SFC separation to give tert-butyl (1R,6R)-6-(4-amino-3-methylphenyl)-7,7-difluoro-3-azabicyclo[4.1.0]heptane-3-carboxylate (200 mg, yield 47%) as a white solid, LC-MS (ESI): mass calcd. for C₁₈H₂₄F₂N₂O₂, 338.18; m/z found, 339 [M+H]⁺; and tert-butyl (1S,6S)-6-(4-amino-3-methylphenyl)-7,7-difluoro-3-azabicyclo[4.1.0]heptane-3-carboxylate (200 mg, yield 47%) as a white solid. LC-MS (ESI): mass calcd. for C₁₈H₂₄F₂N₂O₂, 338.18; m/z found, 339 [M+H]⁺.

### Preparative Separation Method

Instrument: Waters Thar 80 preparative SFC
Column: ChiralPak IC, 250×21.2 mm I.D., 5 μm
Mobile phase: A for C₀₂ and B for MEOH+0.1% NH₃H2O
Gradient: B 35%
Flow rate: 40 mL/min
Back pressure: 100 bar
Column temperature: 35° C.
Wavelength: 220 nm
Cycle-time: 2 min
Eluted time: 3H

### Step F: tert-butyl (1R,6R)-6-(4-(((R)-2,6-dioxopiperidin-3-yl)amino)-3-methylphenyl)-7,7-difluoro-3-azabicyclo[4.1.0]heptane-3-carboxylate & tert-butyl (1R,6R)-6-(4-(((S)-2,6-dioxopiperidin-3-yl)amino)-3-methylphenyl)-7,7-difluoro-3-azabicyclo[4.1.0]heptane-3-carboxylate

To a mixture of tert-butyl (1R,6R)-6-(4-amino-3-methylphenyl)-7,7-difluoro-3-azabicyclo[4.1.0]heptane-3-carboxylate (200 mg, 1 eq, 0.59 mmol) and Sodium bicarbonate (149 mg, 3 eq, 1.77 mmol) in MeCN (5 mL) was added 3-bromopiperidine-2,6-dione (170 mg, 1.5 eq, 0.887 mmol) at room temperature. The mixture was stirred at 80° C. under N₂ atmosphere for 48 h. The mixture was diluted with water (10 mL) and extracted with EA (20 mL×3). The combined organic layers were washed with brine (20 mL), dried over anhydrous Na₂SO₄ and concentrated under reduced pressure. The residue was purified by flash chromatography and eluted with 0-40% DCM in EA and SFC separation to give tert-butyl (1R,6R)-6-(4-(((R)-2,6-dioxopiperidin-3-yl)amino)-3-methylphenyl)-7,7-difluoro-3-azabicyclo[4.1.0]heptane-3-carboxylate (80 mg, yield 30%) as a blue solid, LC-MS (ESI): mass calcd. for C₂₃H₂₉F₂N₃O₄, 449.21; m/z found, 450 [M+H]⁺; and tert-butyl (1R,6R)-6-(4-(((S)-2,6-dioxopiperidin-3-yl)amino)-3-methylphenyl)-7,7-difluoro-3-azabicyclo[4.1.0]heptane-3-carboxylate (80 mg, yield 30%) as a blue solid. LC-MS (ESI): mass calcd. for C₂₃H₂₉F₂N₃O₄, 449.21; m/z found, 450 [M+H]⁺.

### Preparative Separation Method

Instrument: SHIMADZU PREP SOLUTION SFC
Column: ChiralPak IH, 250×21.2 mm I.D., 5 μm
Mobile phase: A for CO₂ and B for MEOH
Gradient: B 20%
Flow rate: 40 mL/min
Back pressure: 100 bar
Column temperature: 40° C.
Wavelength: 220 nm
Cycle-time: 12 min
Eluted time: 2.5 h

### Step G: (R)-3-((4-((1R,6R)-7,7-difluoro-3-azabicyclo[4.1.0]heptan-6-yl)-2-methylphenyl)amino)piperidine-2,6-dione

A mixture of tert-butyl (1R,6R)-6-(4-(((R)-2,6-dioxopiperidin-3-yl)amino)-3-methylphenyl)-7,7-difluoro-3-azabicyclo[4.1.0]heptane-3-carboxylate (80 mg, 1 eq, 0.178 mmol) and HCl (4 M in dioxane; 0.5 mL, 11.2 eq, 2 mmol) in DCM (5 mL) was stirred at room temperature for 16 h. The reaction was monitored by LCMS. Then the precipitate was collected by filtration to afford (R)-3-((4-((1R,6R)-7,7-difluoro-3-azabicyclo[4.1.0]heptan-6-yl)-2-methylphenyl)amino)piperidine-2,6-dione HCl salt (52 mg, yield 76%) as a blue solid. LC-MS (ESI): mass calcd. for C₁₈H₂₁F₂N₃O₂, 349.16; m/z found, 350 [M+H]⁺.

¹H NMR (400 MHz, MeOD-d₄) δ 7.19-7.12 (m, 2H), 6.81 (d, J=8.0 Hz, 1H), 4.40 (dd, J=12.5, 5.0 Hz, 1H), 3.89-3.78 (m, 1H), 3.40-3.35 (m, 1H), 3.26-3.17 (m, 1H), 3.00-2.90 (m, 1H), 2.89-2.78 (m, 1H), 2.76-2.70 (m, 1H), 2.52-2.46 (m, 1H), 2.40-2.29 (m, 2H), 2.25 (s, 3H), 2.21-2.10 (m, 1H), 2.05-1.96 (m, 1H).

¹⁹F NMR (376 MHz, MeOD-d₄) δ −132.54 (d, J=156.7 Hz), −144.75 (d, J=156.6 Hz).

### Step H: (S)-3-((4-((1R,6R)-7,7-difluoro-3-azabicyclo[4.1.0]heptan-6-yl)-2-methylphenyl)amino)piperidine-2,6-dione

A mixture of tert-butyl (1R,6R)-6-(4-(((S)-2,6-dioxopiperidin-3-yl)amino)-3-methylphenyl)-7,7-difluoro-3-azabicyclo[4.1.0]heptane-3-carboxylate (80 mg, 1 eq, 0.178 mmol) and HCl (4 M in dioxane; 0.5 mL, 11.2 eq, 2 mmol) in DCM (5 mL) was stirred at room temperature for 16 h. The reaction was monitored by LCMS. Then the precipitate was collected by filtration to afford (S)-3-((4-((1R,6R)-7,7-difluoro-3-azabicyclo[4.1.0]heptan-6-yl)-2-methylphenyl)amino)piperidine-2,6-dione HCl salt (49 mg, yield 72%) as a blue solid. LC-MS (ESI): mass calcd. for C₁₈H₂₁F₂N₃O₂, 349.16; m/z found, 350 [M+H]⁺.

¹H NMR (400 MHz, MeOD-d₄) δ 7.19-7.12 (m, 2H), 6.81 (d, J=8.0 Hz, 1H), 4.40 (dd, J=12.5, 5.0 Hz, 1H), 3.88-3.80 (m, 1H), 3.41-3.34 (m, 1H), 3.26-3.17 (m, 1H), 2.98-2.88 (m, 1H), 2.87-2.78 (m, 1H), 2.76-2.68 (m, 1H), 2.55-2.45 (m, 1H), 2.40-2.26 (m, 2H), 2.25 (s, 3H), 2.21-2.10 (m, 1H), 2.05-1.92 (m, 1H).

¹⁹F NMR (376 MHz, MeOD-d₄) δ −132.59 (d, J=156.4 Hz), −144.72 (d, J=156.6 Hz).

### Step I: tert-butyl (1S,6S)-6-(4-(((R)-2,6-dioxopiperidin-3-yl)amino)-3-methylphenyl)-7,7-difluoro-3-azabicyclo[4.1.0]heptane-3-carboxylate & tert-butyl (1S,6S)-6-(4-(((S)-2,6-dioxopiperidin-3-yl)amino)-3-methylphenyl)-7,7-difluoro-3-azabicyclo[4.1.0]heptane-3-carboxylate

To a mixture of tert-butyl (1S,6S)-6-(4-amino-3-methylphenyl)-7,7-difluoro-3-azabicyclo[4.1.0]heptane-3-carboxylate (200 mg, 1 eq, 0.59 mmol) and sodium bicarbonate (149 mg, 3 eq, 1.77 mmol) in MeCN (5 mL) was added 3-bromopiperidine-2,6-dione (148 mg, 1.3 eq, 0.768 mmol) at room temperature. The mixture was stirred at 80° C. under N₂ atmosphere for 48 h. Then the mixture was diluted with water (10 mL) and extracted with EA (20 mL×3). The combined organic layers were washed with brine (20 mL), dried over anhydrous Na₂SO₄ and concentrated under reduced pressure. The residue was purified by flash chromatography and eluted 0-40% DCM in EA, then separation by SFC to afford tert-butyl (1S,6S)-6-(4-(((R)-2,6-dioxopiperidin-3-yl)amino)-3-methylphenyl)-7,7-difluoro-3-azabicyclo[4.1.0]heptane-3-carboxylate (80 mg, yield 30%) as a blue solid, LC-MS (ESI): mass calcd. for C₂₃H₂₉F₂N₃O₄, 449.21; m/z found, 450 [M+H]⁺; and tert-butyl (1S,6S)-6-(4-(((S)-2,6-dioxopiperidin-3-yl)amino)-3-methylphenyl)-7,7-difluoro-3-azabicyclo[4.1.0]heptane-3-carboxylate (80 mg, yield 30%) as a blue solid. LC-MS (ESI): mass calcd. for C₂₃H₂₉F₂N₃O₄, 449.21; m/z found, 450 [M+H]⁺.

### Preparative Separation Method

Instrument: SHIMADZU PREP SOLUTION SFC
Column: ChiralCel OZ, 250×21.2 mm I.D., 5 μm
Mobile phase: A for CO₂ and B for MeOH
Gradient: B 40%
Flow rate: 40 mL/min
Back pressure: 100 bar
Column temperature: 35° C.
Wavelength: 254 nm
Cycle-time: 15 min
Eluted time: 3 h

### Step J: (R)-3-((4-((1S,6S)-7,7-difluoro-3-azabicyclo[4.1.0]heptan-6-yl)-2-methylphenyl)amino)piperidine-2,6-dione

A mixture of tert-butyl (1S,6S)-6-(4-(((R)-2,6-dioxopiperidin-3-yl)amino)-3-methylphenyl)-7,7-difluoro-3-azabicyclo[4.1.0]heptane-3-carboxylate (80 mg, 1 eq, 0.178 mmol) and HCl (4 M in dioxane; 0.5 mL, 11.2 eq, 2 mmol) in DCM (5 mL) was stirred at room temperature for 16 h. The reaction was monitored by LCMS. Then the precipitate was collected by filtration to afford (R)-3-((4-((1S,6S)-7,7-difluoro-3-azabicyclo[4.1.0]heptan-6-yl)-2-methylphenyl)amino)piperidine-2,6-dione HCl salt (59 mg, yield 86%) as a blue solid. LC-MS (ESI): mass calcd. for C₁₈H₂₁F₂N₃O₂, 349.16; m/z found, 350 [M+H]⁺.

¹H NMR (400 MHz, MeOD-d₄) δ 7.39-7.28 (m, 2H), 7.08 (d, J=8.3 Hz, 1H), 4.52 (dd, J=12.8, 5.1 Hz, 1H), 3.86 (dd, J=13.8, 9.1 Hz, 1H), 3.38-3.35 (m, 1H), 3.28-3.22 (m, 1H), 3.01-2.91 (m, 1H), 2.85-2.69 (m, 2H), 2.56-2.48 (m, 1H), 2.45-2.38 (m, 1H), 2.36 (s, 3H), 2.33-2.23 (m, 1H), 2.23-2.17 (m, 1H), 2.14-2.01 (m, 1H).

¹⁹F NMR (376 MHz, MeOD-d₄) δ −132.42 (d, J=156.8 Hz), −144.76 (d, J=156.8 Hz).

### Step K: (S)-3-((4-((1S,6S)-7,7-difluoro-3-azabicyclo[4.1.0]heptan-6-yl)-2-methylphenyl)amino)piperidine-2,6-dione

A mixture of tert-butyl (1S,6S)-6-(4-(((S)-2,6-dioxopiperidin-3-yl)amino)-3-methylphenyl)-7,7-difluoro-3-azabicyclo[4.1.0]heptane-3-carboxylate (80 mg, 1 eq, 0.178 mmol) and HCl (4 M in dioxane; 0.5 mL, 11.2 eq, 2 mmol) in DCM (5 mL) was stirred at room temperature for 16 h. The reaction was monitored by LCMS. Then the precipitate was collected by filtration to afford (S)-3-((4-((1S,6S)-7,7-difluoro-3-azabicyclo[4.1.0]heptan-6-yl)-2-methylphenyl)amino)piperidine-2,6-dione HCl salt (57 mg, yield 83%) as a blue solid. LC-MS (ESI): mass calcd. for C₁₈H₂₁F₂N₃O₂, 349.16; m/z found, 350 [M+H]⁺.

¹H NMR (400 MHz, MeOD-d₄) δ 7.36-7.27 (m, 2H), 7.05 (d, J=8.3 Hz, 1H), 4.51 (dd, J=12.7, 5.1 Hz, 1H), 3.86 (dd, J=13.9, 9.3 Hz, 1H), 3.38-3.35 (m, 1H), 3.28-3.21 (m, 1H), 3.01-2.91 (m, 1H), 2.84-2.69 (m, 2H), 2.57-2.48 (m, 1H), 2.43-2.38 (m, 1H), 2.35 (s, 3H), 2.32-2.24 (m, 1H), 2.23-2.14 (m, 1H), 2.13-2.01 (m, 1H).

¹⁹F NMR (376 MHz, MeOD-d₄) δ −132.43 (d, J=156.8 Hz), −144.74 (d, J=156.8 Hz).

### Compound 11. (R)-3-((2H-spiro[benzofuran-3,4′-piperidin]-6-yl)amino)piperidine-2,6-dione

### Compound 12. (S)-3-((2H-spiro[benzofuran-3,4′-piperidin]-6-yl)amino)piperidine-2,6-dione

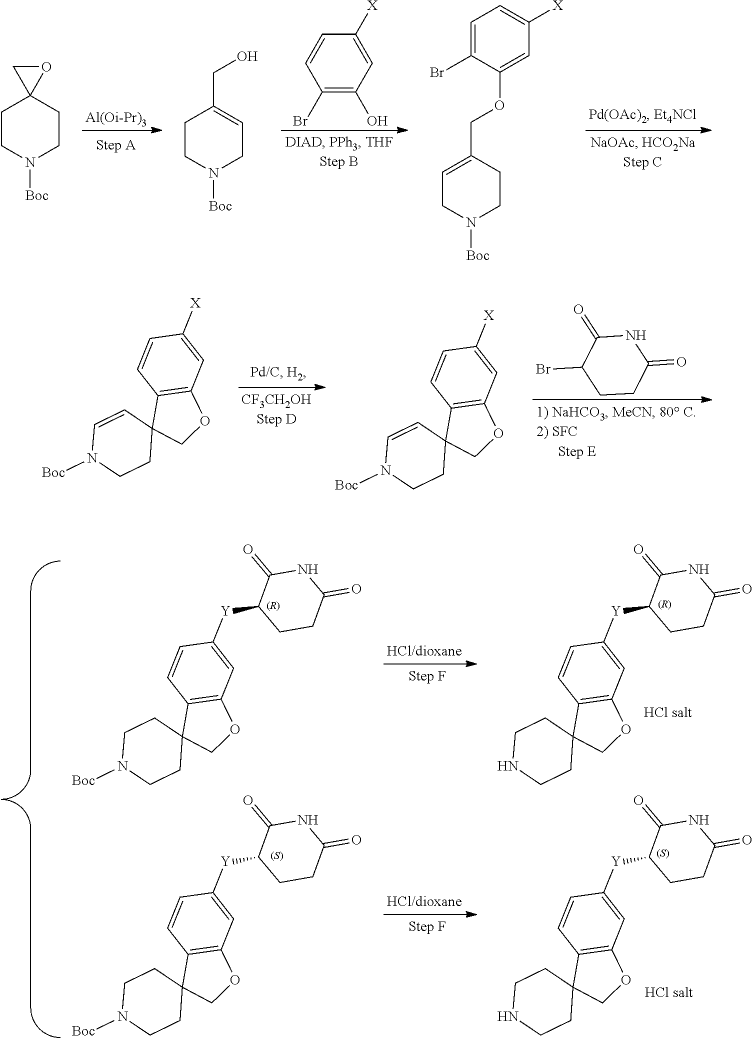

### Step A: tert-butyl 4-(hydroxymethyl)-3,6-dihydropyridine-1(2H)-carboxylate

A mixture of tert-butyl 1-oxa-6-azaspiro[2.5]octane-6-carboxylate (50 g, 234 mmol, 1 eq.) and aluminum isopropoxide (92.5 mL, 469 mmol, 2 eq.) in toluene (800 mL) was stirred at 110° C. under N₂ atmosphere for 20 hours. Then the mixture was diluted with 1 N HCl (800 mL) and extracted with EA (1 L×2). The combined organic layers were washed with brine (800 mL), dried over anhydrous Na₂SO₄ and concentrated under reduced pressure. The residue was purified by flash chromatography and eluted with 25-50% EA in PE to give tert-butyl 4-(hydroxymethyl)-3,6-dihydropyridine-1(2H)-carboxylate (17 g, yield 34%) as a yellow oil.

¹H NMR (400 MHz, CDCl₃) δ 5.65 (s, 1H), 4.04 (s, 2H), 3.90 (d, J=2.1 Hz, 2H), 3.51 (t, J=5.7 Hz, 2H), 2.12 (s, 2H), 1.47 (d, J=2.3 Hz, 9H).

### Step B: tert-butyl 4-((2-bromo-5-nitrophenoxy)methyl)-3,6-dihydropyridine-1(2H)-carboxylate

To a solution of 2-bromo-5-nitrophenol (12.0 g, 55.0 mmol, 1 eq.), tert-butyl 4-(hydroxymethyl)-3,6-dihydropyridine-1(2H)-carboxylate (12.9 g, 60.5 mmol, 1.1 eq.) and triphenylphosphane (17.3 g, 66.1 mmol, 1.2 eq.) in THF (200 mL) was slowly added DIAD (13 mL, 66.1 mmol, 1.2 eq.) at 0° C. Then the reaction mixture was stirred at room temperature under N₂ atmosphere for 16 hours. Then the mixture was diluted with water (300 mL) and extracted with EA (300 mL×2). The combined organic layers were washed with brine (300 mL), dried over anhydrous Na₂SO₄ and concentrated under reduced pressure. The residue was purified by flash chromatography and eluted with 5-20% EA in PE to give tert-butyl 4-((2-bromo-5-nitrophenoxy)methyl)-3,6-dihydropyridine-1(2H)-carboxylate (17 g, yield 75%) as a yellow solid.

¹H NMR (400 MHz, MeOD-d₄) δ 7.84 (d, J=2.4 Hz, 1H), 7.81-7.78 (m, 1H), 7.74 (dd, J=8.7, 2.4 Hz, 1H), 5.93 (s, 1H), 4.68 (s, 2H), 3.95 (s, 2H), 3.56 (t, J=5.3 Hz, 2H), 2.25 (d, J=1.7 Hz, 2H), 1.47 (s, 9H).

### Step C: tert-butyl 6-nitro-2′,3′-dihydro-1′H,2H-spiro[benzofuran-3,4′-pyridine]-1′-carboxylate

A solution of tert-butyl 4-((2-bromo-5-nitrophenoxy)methyl)-3,6-dihydropyridine-1(2H)-carboxylate (10 g, 24.2 mmol, 1 eq.), sodium acetate (4.96 g, 60.5 mmol, 2.5 eq.), tetraethylammonium chloride (4.41 g, 26.6 mmol, 1.1 eq.), sodium formate (1.81 g, 26.6 mmol, 1.1 eq.) and palladium diacetate (543 mg, 2.42 mmol, 0.1 eq.) in DMF (200 mL) was stirred at 70° C. under N₂ atmosphere for 16 hours. Then the reaction was filtered through a Celite pad and the filtrate was diluted with water (600 mL) and extracted with EA (600 mL×2). The combined organic layers were washed with brine (600 mL), dried over anhydrous Na₂SO₄ and concentrated under reduced pressure. The residue was purified by flash chromatography and eluted with 5-25% EA in PE to give tert-butyl 6-nitro-2′,3′-dihydro-1′H,2H-spiro[benzofuran-3,4′-pyridine]-1′-carboxylate (6 g, yield 75%) as a yellow solid.

¹H NMR (400 MHz, MeOD-d₄) δ 7.81 (dd, J=8.2, 2.1 Hz, 1H), 7.58 (d, J=2.0 Hz, 1H), 7.29 (d, J=8.2 Hz, 1H), 7.15-7.15 (m, 1H), 4.91-4.81 (m, 1H), 4.51 (d, J=9.1 Hz, 1H), 4.38 (d, J=9.1 Hz, 1H), 3.90-3.86 (m, 1H), 3.53-3.43 (m, 1H), 2.06-2.01 (m, 1H), 1.92-1.82 (m, 1H), 1.51 (s, 9H).

### Step D: tert-butyl 6-amino-2H-spiro[benzofuran-3,4′-piperidine]-1′-carboxylate

A mixture of tert-butyl 6-nitro-2′,3′-dihydro-1′H,2H-spiro[benzofuran-3,4′-pyridine]-1′-carboxylate (6 g, 18.1 mmol) and 10% Pd/C (800 mg) in CF₃CH₂OH (60 mL) was stirred at room temperature under H₂ atmosphere (1 atm) for 16 hours. The mixture was filtered through a Celite pad and the filtrate was concentrated under reduced pressure. The residue was purified by flash chromatography and eluted with 5-25% EA in PE to give tert-butyl 6-amino-2H-spiro[benzofuran-3,4′-piperidine]-1′-carboxylate (4 g, yield 73%) as a yellow solid.

¹H NMR (400 MHz, MeOD-d₄) δ 6.86 (d, J=8.0 Hz, 1H), 6.25 (dd, J=8.0, 2.0 Hz, 1H), 6.16 (d, J 1.9 Hz, 1H), 4.35 (s, 2H), 3.98 (dt, J=13.8, 3.6 Hz, 2H), 2.97 (s, 2H), 1.80-1.70 (m, 2H), 1.63 (d, J=13.6 Hz, 2H), 1.47 (s, 9H).

### Step E: tert-butyl (R)-6-((2,6-dioxopiperidin-3-yl)amino)-2H-spiro[benzofuran-3,4′-piperidine]-1′-carboxylate & tert-butyl (S)-6-((2,6-dioxopiperidin-3-yl)amino)-2H-spiro[benzofuran-3,4′-piperidine]-1′-carboxylate

A solution of tert-butyl 6-amino-2H-spiro[benzofuran-3,4′-piperidine]-1′-carboxylate (4 g, 13.1 mmol, 1 eq.), 3-bromopiperidine-2,6-dione (4.54 g, 23.7 mmol, 1.8 eq.) and Sodium bicarbonate (3.31 g, 39.4 mmol, 3 eq.) in MeCN (70 mL) was stirred at 90° C. for 56 hours. The reaction was filtered, and the filtrate was concentrated under reduced pressure to give a residue. The residue was purified by reversed-phase column (C18, H₂O/MeCN/0.1% FA) to give tert-butyl 6-((2,6-dioxopiperidin-3-yl)amino)-2H-spiro[benzofuran-3,4′-piperidine]-1′-carboxylate (2.2 g). Then the product was separated by SFC to give tert-butyl (R)-6-((2,6-dioxopiperidin-3-yl)amino)-2H-spiro[benzofuran-3,4′-piperidine]-1′-carboxylate (0.6 g, yield 11%) and tert-butyl (S)-6-((2,6-dioxopiperidin-3-yl)amino)-2H-spiro[benzofuran-3,4′-piperidine]-1′-carboxylate (0.65 g, yield 12%). LC-MS (ESI): mass calcd. for C₂₂H₂₉N₃O₅, 415.21; m/z found, 416 [M+H]⁺.

### Preparative Separation Method:

Instrument: Waters Thar 80 preparative SFC
Column: (R,R)-WHELK, 250×21.2 mm I.D., 5 μm
Mobile phase: A for CO₂ and B for MEOH
Gradient: B 35%
Flow rate: 40 mL/min
Back pressure: 100 bar
Column temperature: 35° C.
Wavelength: 244 nm
Cycle-time: 14 min
Eluted time: 4H

### Step F: (R)-3-((2H-spiro[benzofuran-3,4′-piperidin]-6-yl)amino)piperidine-2,6-dione HCl salt

To a solution of tert-butyl (R)-6-((2,6-dioxopiperidin-3-yl)amino)-2H-spiro[benzofuran-3,4′-piperidine]-1′-carboxylate (500 mg, 1.20 mmol) in DCM (5 mL) was added HCl/dioxane (4 M, 20 mL). Then the reaction was stirred at room temperature for 16 hours. The reaction was concentrated under reduced pressure to give a residue. The residue was purified by Prep-HPLC to give (R)-3-((2H-spiro[benzofuran-3,4′-piperidin]-6-yl)amino)piperidine-2,6-dione HCl salt (240 mg, yield 57%) as a yellow solid. LC-MS (ESI): mass calcd. for C₁₇H₂₁N₃O₃, 315.16; m/z found, 316 [M+H]⁺.

¹H NMR (400 MHz, DMSO-d₆) δ 10.79 (s, 1H), 9.11 (bs, 1H), 8.99 (bs, 1H), 6.85 (d, J=8.1 Hz, 1H), 6.26 (dd, J=8.1, 1.8 Hz, 1H), 6.20 (d, J=1.7 Hz, 1H), 4.38 (s, 2H), 4.30 (dd, J=11.4, 4.8 Hz, 1H), 3.25 (d, J=12.8 Hz, 2H), 2.98-2.89 (m, 2H), 2.80-2.68 (m, 1H), 2.61-2.53 (m, 1H), 2.10-1.94 (m, 3H), 1.91-1.81 (m, 1H), 1.74 (d, J=14.0 Hz, 2H).

### Step G: (S)-3-((2H-spiro[benzofuran-3,4′-piperidin]-6-yl)amino)piperidine-2,6-dione HCl salt

A solution of tert-butyl (S)-6-((2,6-dioxopiperidin-3-yl)amino)-2H-spiro[benzofuran-3,4′-piperidine]-1′-carboxylate (650 mg, 1.56 mmol) in HCl/dioxane (4 M, 20 mL) was stirred at room temperature for 16 hours. The reaction was concentrated under reduced pressure to give a residue. Then the precipitate was collected by filtration to afford (S)-3-((2H-spiro[benzofuran-3,4′-piperidin]-6-yl)amino)piperidine-2,6-dione HCl salt (540 mg, yield 98%) as a yellow solid. LC-MS (ESI): mass calcd. for C₁₇H₂₁N₃O₃, 315.16; m/z found, 316 [M+H]⁺.

¹H NMR (400 MHz, DMSO-d₆) δ 10.79 (s, 1H), 9.05 (bs, 1H), 8.91 (bs, 1H), 6.84 (d, J=8.1 Hz, 1H), 6.25 (dd, J=8.2, 1.9 Hz, 1H), 6.19 (d, J=1.9 Hz, 1H), 4.38 (s, 2H), 4.29 (dd, J=11.4, 4.8 Hz, 1H), 3.26 (d, J=13.0 Hz, 2H), 2.98-2.89 (m, 2H), 2.80-2.68 (m, 1H), 2.61-2.54 (m, 1H), 2.12-1.94 (m, 3H), 1.92-1.82 (m, 1H), 1.74 (d, J=14.0 Hz, 2H).

### Compound 13. tert-butyl (4aR)-8-((2,6-dioxopiperidin-3-yl)amino)-1,2,4a,5-tetrahydrobenzo[b]pyrazino[1,2-d][1,4]oxazine-3(41)-carboxylate

### Compound 14. 3—(((R)-1,2,3,4,4a,5-hexahydrobenzo[b]pyrazino[1,2-d][1,4]oxazin-8-yl)amino)piperidine-2,6-dione hydrochloride salt

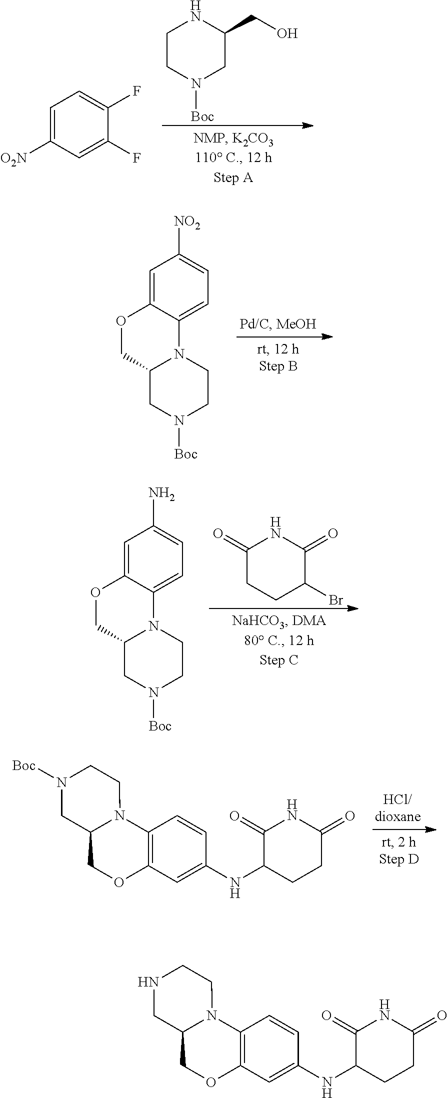

### Step A: tert-butyl (R)-8-nitro-1,2,4a,5-tetrahydrobenzo[b]pyrazino[1,2-d][1,4]oxazine-3(4H)-carboxylate

To a mixture of 1,2-difluoro-4-nitrobenzene (5.1 g, 32.4 mol, 1.0 eq.) and tert-butyl (R)-3-(hydroxymethyl)piperazine-1-carboxylate (7.0 g, 32.4 mol, 1.0 eq.) in DMA (50 mL) was added Cs₂CO₃ (21.0 g, 64.8 mol, 2.0 eq). The mixture was stirred at 110° C. for 12h, cooled to room temperature, and poured into ice-water (500 mL). The precipitate was collected by filtration and purified by column chromatography on silica gel eluted with 0-50% EtOAc/hexane to afford tert-butyl (R)-8-nitro-1,2,4a,5-tetrahydrobenzo[b]pyrazino[1,2-d][1,4]oxazine-3(4H)-carboxylate (3.5 g, 32.2% crude yield). LC-MS (ESI): mass calcd. for C₁₆H₂₁N₃O₅, 335.15; m/z found, 336.4 [M+H]⁺.

### Step B: tert-butyl (R)-8-amino-1,2,4a,5-tetrahydrobenzo[b]pyrazino[1,2-d][1,4]oxazine-3(4H)-carboxylate

To a mixture of tert-butyl (R)-8-nitro-1,2,4a,5-tetrahydrobenzo[b]pyrazino[1,2-d][1,4]oxazine-3(4H)-carboxylate (2.5 g, 7.4 mmol, 1 eq.) in MeOH (30 mL) was added Pd/C (250 mg, 10% on Carbon, wetted with ca. 55% water) and the reaction mixture was stirred at room temperature for 12 h. The catalyst was removed by filtration and the filtrate was concentrated to afford tert-butyl 4-(4-amino-2-fluorophenyl)piperazine-1-carboxylate (2.2 g, crude) as a brown solid. LC-MS (ESI): mass calcd. for C₁₆H₂₃N₃O₃, 305.17; m/z found, 306.4 [M+H]⁺.

### Step C: tert-butyl (4aR)-8-((2,6-dioxopiperidin-3-yl)amino)-1,2,4a,5-tetrahydrobenzo[b]pyrazino[1,2-d][1,4]oxazine-3(4H)-carboxylate

To a mixture of tert-butyl (R)-8-amino-1,2,4a,5-tetrahydrobenzo[b]pyrazino[1,2-d][1,4]oxazine-3(4H)-carboxylate (0.7 g, 2.3 mmol 1 eq.) and 3-bromopiperidine-2,6-dione (440 mg, 2.3 mmol, I eq.) in DMA (10 mL) was added NaHCO₃ (193 mg, 2.3 mmol, 1 eq.). The reaction mixture was stirred at 80° C. for 12h, cooled to room temperature and then poured into water (100 mL). The precipitate was collected by filtration and dried in vacuo to afford tert-butyl (4aR)-8-((2,6-dioxopiperidin-3-yl)amino)-1,2,4a,5-tetrahydrobenzo[b]pyrazino[1,2-d][1,4]oxazine-3(4H)-carboxylate (530 mg, 55.4% yield) as green solid. LC-MS (ESI): mass calcd. for C₂₁H₂₈N₄O₅, 416.21; m/z found, 417.2 [M+H]⁺. ¹H NMR (400 MHz, DMSO) δ 10.73 (s, 1H), 6.66 (d, J=8.8 Hz, 1H), 6.20 (d, J=8.8 Hz, 1H), 6.14 (s, 1H), 5.33 (d, J=7.2 Hz, 1H), 4.24-4.13 (m, 2H), 3.96-3.80 (m, 3H), 3.63-3.61 (m, 1H), 2.74-2.56 (m, 2H), 2.39-2.32 (m, 2H), 2.07-2.05 (m, 1H), 1.82-1.81 (m, 1H), 1.41 (s, 9H).

### Step D: 3-(((R)-1,2,3,4,4a,5-hexahydrobenzo[b]pyrazino[1,2-d][1,4]oxazin-8-yl)amino)piperidine-2,6-dione hydrochloride salt

A mixture of tert-butyl (4aR)-8-((2,6-dioxopiperidin-3-yl)amino)-1,2,4a,5-tetrahydrobenzo[b]pyrazino[1,2-d][1,4]oxazine-3(4H)-carboxylate (50.0 mg, 0.12 mmol, 1.0 eq.) in HCl/dioxane (1 mL) was stirred at room temperature for 2 h. The reaction mixture was concentrated to afford 3-(((R)-1,2,3,4,4a,5-hexahydrobenzo[b]pyrazino[1,2-d][1,4]oxazin-8-yl)amino)piperidine-2,6-dione hydrochloride salt (42 mg, 100% yield) as brown solid. LC-MS (ESI): mass calcd. for C₁₆H₂₀N₄O₃, 316.15; m/z found, 317.3 [M+H]⁺. ¹H NMR (400 MHz, DMSO) δ 10.99 (s, 1H), 9.51 (d, J=16 Hz, 2H), 6.84 (d, J=7.6 Hz, 2H), 6.51 (d, J=25.2 Hz, 2H), 4.36-4.24 (m, 2H), 3.96-3.86 (m, 2H), 3.93-3.31 (m, 3H), 3.04-2.59 (m, 5H), 1.96-1.91 (m, 2H).

### Compound 15. 3-((3H-spiro[isobenzofuran-1,4′-piperidin]-5-yl)amino)piperidine-2,6-dione HCO₂H salt

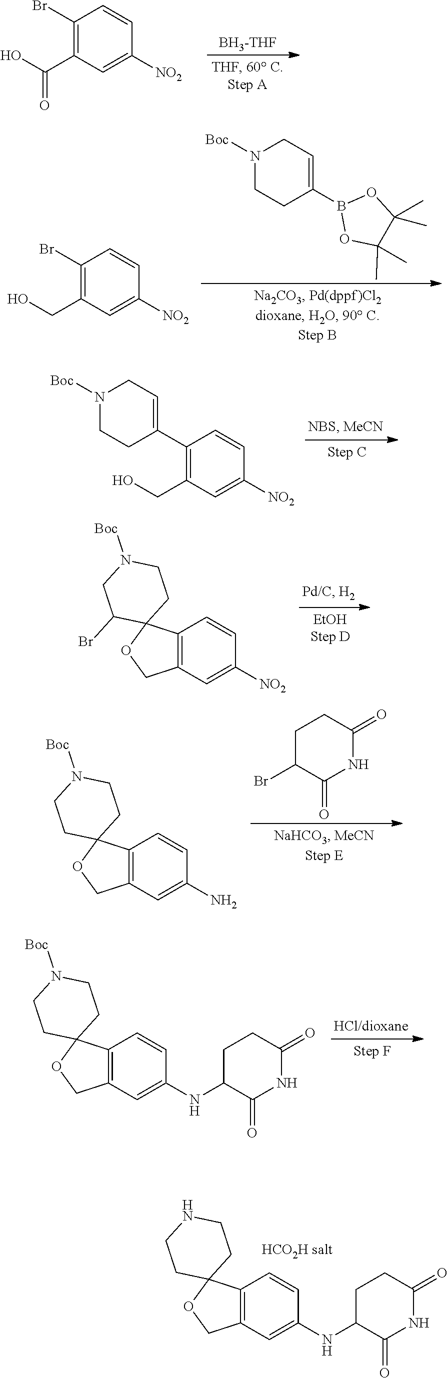

### Step A: (2-bromo-5-nitrophenyl)methanol

To a solution of 2-bromo-5-nitrobenzoic acid (2 g, 8.13 mmol, 1.0 eq.) in THF (5 mL) was added BH₃ (1 M in THF, 16.3 mL, 16.3 mmol, 2.0 eq.) at 0° C. The mixture solution was stirred at 60° C. for 2 hours. After quenched with water (10 mL) at room temperature, the solution was adjusted to pH~1 with 6 N HCl aq. and extracted with EA (20×3 mL). The combined organic layers were washed with brine (20 mL), dried over anhydrous Na₂SO₄ and concentrated under reduced pressure. The residue was purified by flash chromatography and eluted with 0-30% EA in PE to give (2-bromo-5-nitrophenyl)methanol (1.8 g, yield 95%) as a yellow oil. LC-MS (ESI): mass calcd. For C₇H₆BrNO₃, 230.95; m/z found, 232.1 [M+H]⁺.

### Step B: tert-butyl 4-(2-(hydroxymethyl)-4-nitrophenyl)-3,6-dihydropyridine-1(2H)-carboxylate

A mixture of (2-bromo-5-nitrophenyl)methanol (1.9 g, 8.19 mmol, 1.0 eq.), tert-butyl 4-(4,4,5,5-tetramethyl-1,3,2-dioxaborolan-2-yl)-3,6-dihydropyridine-1(2H)-carboxylate (3.04 g, 9.83 mmol, 1.2 eq.), Pd(dppf)Cl₂ (599 mg, 819 smol, 0.1 eq.), Na₂CO₃ (2.6 g, 24.6 mmol, 3.0 eq.) in 1,4-dioxane (20 mL) and H₂O (2 mL) was stirred at 90° C. under N₂ atmosphere for 12 hours. Then the mixture was diluted with water (50 mL) and extracted with EA (50 mL×2). The combined organic layers were washed with brine (50 mL), dried over anhydrous Na₂SO₄ and concentrated under reduced pressure. The residue was purified by flash chromatography and eluted with 10-50% EA in PE to give tert-butyl 4-(2-(hydroxymethyl)-4-nitrophenyl)-3,6-dihydropyridine-1(2H)-carboxylate (2.5 g, yield 91%) as a brown oil. LC-MS (ESI): mass calcd. for C₁₇H₂₂N₂O₅, 334.15; m/z found, 335.1 [M+H]⁺.

### Step C: tert-butyl 3′-bromo-5-nitro-3H-spiro[isobenzofuran-1,4′-piperidine]-1′-carboxylate

A mixture of tert-butyl 4-(2-(hydroxymethyl)-4-nitrophenyl)-3,6-dihydropyridine-1(2H)-carboxylate (1 g, 2.99 mmol, 1.0 eq.) and NBS (798 mg, 4.49 mmol, 1.5 eq.) in MeCN (15 mL) was stirred at 25° C. for 1 hour. Then the mixture was diluted with sat. Na₂S₂O₃ aq. (30 mL) and extracted with EA (30 mL×2). The combined organic layers were washed with brine (30 mL), dried over anhydrous Na₂SO₄ and concentrated under reduced pressure. The residue was purified by flash chromatography and eluted with 0-20% EA in PE to give tert-butyl 3′-bromo-5-nitro-3H-spiro[isobenzofuran-1,4′-piperidine]-1′-carboxylate (900 mg, yield 73%) as a yellow solid. LC-MS (ESI): mass calcd. For C₁₇H₂₁BrN₂O₅, 412.06; m/z found, 413.2 [M+H]⁺.

### Step D: tert-butyl 5-amino-3H-spiro[isobenzofuran-1,4′-piperidine]-1′-carboxylate

A mixture of tert-butyl 3′-bromo-5-nitro-3H-spiro[isobenzofuran-1,4′-piperidine]-1′-carboxylate (200 mg, 484 μmol) and 10% Pd/C (103 mg) in EtOH (5 mL) was stirred at 25° C. under H₂ atmosphere (1 atm) for 5 hours. The mixture solution was filtrated through a Celite pad and the filtrate was concentrated under reduced pressure. The residue was purified by Prep-TLC (PE/EA=1/1) to afford tert-butyl 5-amino-3H-spiro[isobenzofuran-1,4′-piperidine]-1′-carboxylate (85 mg, yield 58%) as a yellow solid. LC-MS (ESI): mass calcd. for C₁₇H₂₄N₂O₃, 304.18; m/z found, 305.2 [M+H]⁺.

### Step E: tert-butyl 5-((2,6-dioxopiperidin-3-yl)amino)-3H-spiro[isobenzofuran-1,4′-piperidine]-1′-carboxylate

A mixture of tert-butyl 5-amino-3H-spiro[isobenzofuran-1,4′-piperidine]-1′-carboxylate (100 mg, 329 μmol, 1.0 eq.), 3-bromopiperidine-2,6-dione (94.6 mg, 493 μmol, 1.5 eq.) and sodium bicarbonate (28 mg, 329 μmol, 1.0 eq.) in MeCN (10 mL) was stirred at 80° C. for 24 hours. The mixture solution was filtered and concentrated under reduced pressure to give a residue, then purified by Prep-TLC (PE/EA=1/2) to afford tert-butyl 5-((2,6-dioxopiperidin-3-yl)amino)-3H-spiro[isobenzofuran-1,4′-piperidine]-1′-carboxylate (65 mg, yield 48%) as a white solid. LC-MS (ESI): mass calcd. for C₂₂H₂₉N₃O₅, 415.21; m/z found, 416.3 [M+H]⁺.

### Step F: 3-((3H-spiro[isobenzofuran-1,4′-piperidin]-5-yl)amino)piperidine-2,6-dione HCO₂H salt

A mixture of tert-butyl 5-((2,6-dioxopiperidin-3-yl)amino)-3H-spiro[isobenzofuran-1,4′-piperidine]-1′-carboxylate (25 mg, 60.2 μmol) and 4 M HCl in dioxane (1 mL) in DCM (2 mL) was stirred at 25° C. for 1 hour. The mixture solution was concentrated under reduced pressure to give a residue then purified by Prep-HPLC to give 3-((3H-spiro[isobenzofuran-1,4′-piperidin]-5-yl)amino)piperidine-2,6-dione HCO₂H salt (2.9 mg, yield 13%) as a yellow solid. LC-MS (ESI): mass calcd. for C₁₇H₂₁N₃O₃, 315.16; m/z found, 316.1 [M+H]⁺.

¹H NMR (400 MHz, DMSO-d₆) δ 10.76 (bs, 1H), 8.40 (s, 1H, HCO₂H salt), 6.89 (d, J=8.2 Hz, 1H), 6.60 (d, J=8.2 Hz, 1H), 6.55 (s, 1H), 5.91 (d, J=7.5 Hz, 1H), 4.88 (s, 2H), 4.35-4.27 (m, 1H), 3.75 (bs, 1H), 3.10 (d, J=11.7 Hz, 2H), 2.96 (t, J=11.5 Hz, 2H), 2.74 (ddd, J=17.4, 12.0, 5.3 Hz, 1H), 2.62-2.53 (m, 1H), 2.15-2.04 (m, 1H), 1.98-1.81 (m, 3H), 1.62 (d, J=13.4 Hz, 2H).

### Compound 16. 3-((3′,3′-difluoro-2H-spiro[benzofuran-3,4′-piperidin]-6-yl)amino)piperidine-2,6-dione

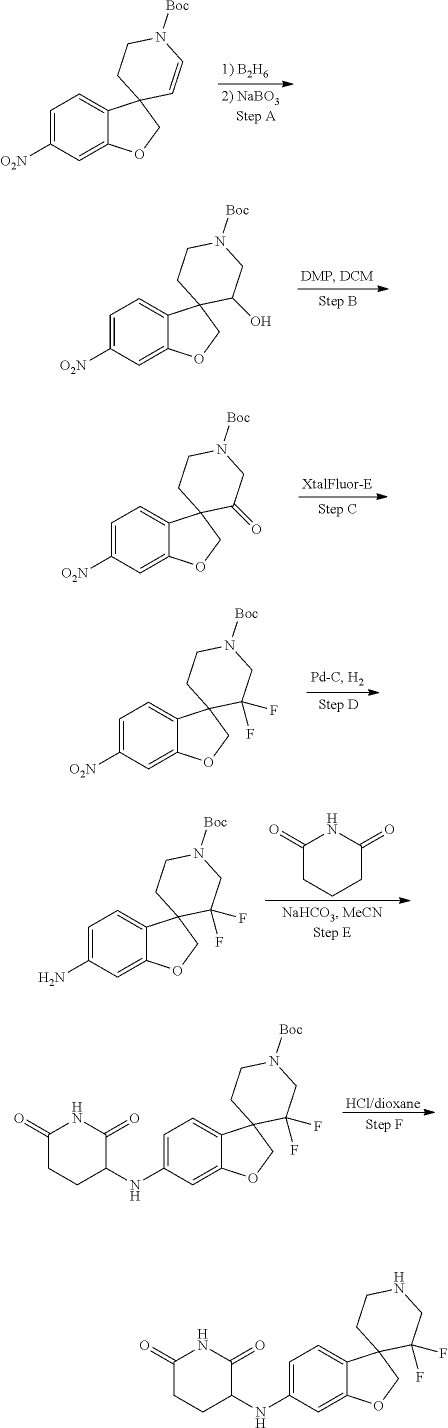

### Step A: tert-butyl 3′-hydroxy-6-nitro-2H-spiro[benzofuran-3,4′-piperidine]-1′-carboxylate

To a mixture of tert-butyl 6-nitro-2′,3′-dihydro-1′H,2H-spiro[benzofuran-3,4′-pyridine]-1′-carboxylate (6.5 g, 19.6 mmol, 1 eq.) in THF (90 mL) was added BH₃-THF (1 M, 35 mL, 35 mmol, 1.8 eq.) at −70° C. The reaction mixture was stirred at this temperature for 0.5 h, and then warmed to 20° C. over 5 hours. Water (25 mL) was added followed by sodium perborate tetrahydrate (15 g, 97.8 mmol, 5 eq.). Then the mixture was stirred at 20° C. overnight.

The mixture was diluted with water (180 mL) and extracted with DCM (200 mL). The separated organic layer was dried over Na₂SO₄, filtered, and concentrated under reduced pressure to afford tert-butyl 3′-hydroxy-6-nitro-2H-spiro[benzofuran-3,4′-piperidine]-1′-carboxylate (6.5 g, crude) as a yellow oil. LC-MS (ESI): mass calced for C₁₇H₂₂N₂O₆, 350.15; m/z found, 295 [M−56+H]⁺.

### Step B: tert-butyl 6-nitro-3′-oxo-2H-spiro[benzofuran-3,4′-piperidine]-1′-carboxylate

A mixture of tert-butyl 3′-hydroxy-6-nitro-2H-spiro[benzofuran-3,4′-piperidine]-1′-carboxylate (6.5 g, 18.6 mmol, 1 eq.) and DMP (12.6 g, 29.7 mmol, 1.6 eq.) in DCM (70 mL) was stirred at 20° C. for 2 hours. The reaction was washed with sat. sodium bisulfite aq. (70 mL) and sat. sodium bicarbonate aq. (70 mL) in sequence. The separated organic phase was dried over Na₂SO₄, filtered, and concentrated under reduced pressure. The residue was purified by flash chromatography and eluted with 0-25% EA in PE to give tert-butyl 6-nitro-3′-oxo-2H-spiro[benzofuran-3,4′-piperidine]-1′-carboxylate (6 g, yield 93%) as a yellow oil. LC-MS (ESI): mass calced for C₁₇H₂₀N₂O₆, 348.13; m/z found, 249.1 [M−100+H]⁺.

¹H NMR (400 MHz, DMSO-d₆) δ 7.80 (dd, J=8.3, 2.1 Hz, 1H), 7.64 (d, J=2.0 Hz, 1H), 7.55 (d, J=8.3 Hz, 1H), 4.90 (d, J=9.5 Hz, 1H), 4.60 (d, J=9.5 Hz, 1H), 4.23 (s, 2H), 3.79-3.71 (m, 1H), 3.56 (s, 1H), 2.44-2.36 (m, 1H), 2.30-2.22 (m, 1H), 1.45 (s, 9H).

### Step C: tert-butyl 3′,3′-difluoro-6-nitro-2H-spiro[benzofuran-3,4′-piperidine]-1′-carboxylate

A mixture of tert-butyl 6-nitro-3′-oxo-2H-spiro[benzofuran-3,4′-piperidine]-1′-carboxylate (5.5 g, 15.8 mmol, 1 eq.), triethylammonium fluoride (15.4 mL, 94.7 mmol, 6 eq.), N,N-Diethyl-S,S-difluoro-sulfiliminium tetrafluoroborate (16.3 g, 71 mmol, 4.5 eq.) and TEA (5.50 mL, 39.5 mmol, 2.5 eq.) in DCM (300 mL) was stirred at 25° C. for 1 hour. Then the mixture was diluted with sat. NaHCO₃ aq. (300 mL) and extracted with DCM (300 mL). The combined organic layers were washed with brine (300 mL), dried over anhydrous Na₂SO₄ and concentrated under reduced pressure. The residue was purified by flash chromatography and eluted with 0-30% EA in PE to give tert-butyl 3′,3′-difluoro-6-nitro-2H-spiro[benzofuran-3,4′-piperidine]-1′-carboxylate (2.3 g, yield 39%) as a yellow oil. LC-MS (ESI): mass calced for C₁₇H₂₀F₂N₂O₅, 370.13; m/z found, 292.9 [M−100+23]⁺.

### Step D: tert-butyl 6-amino-3′,3′-difluoro-2H-spiro[benzofuran-3,4′-piperidine]-1′-carboxylate

A mixture of tert-butyl 3′,3′-difluoro-6-nitro-2H-spiro[benzofuran-3,4′-piperidine]-1′-carboxylate (2.3 g, 6.21 mmol) and 10% Pd/C (500 mg) in THF/MeOH (1:1, 20 mL) was stirred at 25° C. under H₂ atmosphere (1 atm) for 6 hours. The mixture solution was filtrated through a Celite pad and the filtrate was concentrated under reduced pressure to afford tert-butyl 6-amino-3′,3′-difluoro-2H-spiro[benzofuran-3,4′-piperidine]-1′-carboxylate (1.9 g, yield 90%) as a yellow oil. Revised as the following: LC-MS (ESI): mass calced for C₁₇H₂₂F₂N₂O₃, 340.16; m/z found, 341.1 [M+H]⁺.

### Step E: tert-butyl 6-((2,6-dioxopiperidin-3-yl)amino)-3′,3′-difluoro-2H-spiro[benzofuran-3,4′-piperidine]-1′-carboxylate

A mixture of tert-butyl 6-amino-3′,3′-difluoro-2H-spiro[benzofuran-3,4′-piperidine]-1′-carboxylate (300 mg, 881 μmol, 1 eq.), 3-bromopiperidine-2,6-dione (254 mg, 1.32 mmol, 1.5 eq.) and sodium bicarbonate (222 mg, 2.64 mmol, 3 eq.) in MeCN (3 mL) was stirred at 80° C. for 16 hours. The reaction mixture was filtered and concentrated under reduced pressure. The residue was purified by Prep-TLC (EA/PE: 1/1) to tert-butyl 6-((2,6-dioxopiperidin-3-yl)amino)-3′,3′-difluoro-2H-spiro[benzofuran-3,4′-piperidine]-1′-carboxylate (60 mg, yield 15%) as a colorless solid. LC-MS (ESI): mass calced for C₂₂H₂₇F₂N₃O₅, 451.19; m/z found, 452.2 [M+H]⁺.

¹H NMR (400 MHz, DMSO-d₆) δ 10.77 (s, 1H), 7.01 (d, J=8.2 Hz, 1H), 6.22 (d, J=8.3 Hz, 1H), 6.16 (d, J=1.7 Hz, 1H), 5.95 (d, J=7.2 Hz, 1H), 4.66 (d, J=10.1 Hz, 1H), 4.34-4.28 (m, 2H), 4.08-4.04 (m, 1H), 3.89-3.82 (m, 1H), 3.55-3.46 (m, 1H), 3.03-2.95 (m, 1H), 2.79-2.67 (m, 1H), 2.59-2.54 (m, 1H), 2.10-1.99 (m, 2H), 1.91-1.74 (m, 2H), 1.42 (s, 9H).

### Step F: 3-((3′,3′-difluoro-2H-spiro[benzofuran-3,4′-piperidin]-6-yl)amino)piperidine-2,6-dione HCOOH salt

A mixture of tert-butyl 6-((2,6-dioxopiperidin-3-yl)amino)-3′,3′-difluoro-2H-spiro[benzofuran-3,4′-piperidine]-1′-carboxylate (60 mg, 133 μmol) and 4 M HCl in dioxane (0.1 mL) in DCM (1 mL) was stirred at 25° C. for 5 hours. The mixture solution was concentrated under reduced pressure to give a residue then purified by Prep-HPLC to give 3-((3′,3′-difluoro-2H-spiro[benzofuran-3,4′-piperidin]-6-yl)amino)piperidine-2,6-dione HCO₂H salt (5.3 mg, yield 10%) as a white solid. LC-MS (ESI): mass calced for C₁₇H19F2N₃O3, 351.14; m/z found, 352.1 [M+H]⁺.

¹H NMR (400 MHz, DMSO-d₆) δ 10.77 (s, 1H), 8.17 (s, 1H, HCOOH salt), 6.98 (d, J=8.2 Hz, 1H), 6.21 (d, J=8.3 Hz, 1H), 6.14 (d, J=1.9 Hz, 1H), 5.90 (d, J=7.6 Hz, 1H), 4.63 (d, J=9.6 Hz, 1H), 4.32-4.26 (m, 2H), 3.10-3.01 (m, 1H), 2.95-2.81 (m, 2H), 2.78-2.69 (m, 1H), 2.61-2.54 (m, 1H), 2.48-2.35 (m, 1H), 2.13-2.04 (m, 1H), 2.04-1.94 (m, 1H), 1.92-1.80 (m, 1H), 1.75-1.67 (m, 1H).

### Compound 17. 3-((4′-oxo-2H-spiro[benzofuran-3,1′-cyclohexan]-6-yl)amino)piperidine-2,6-dione

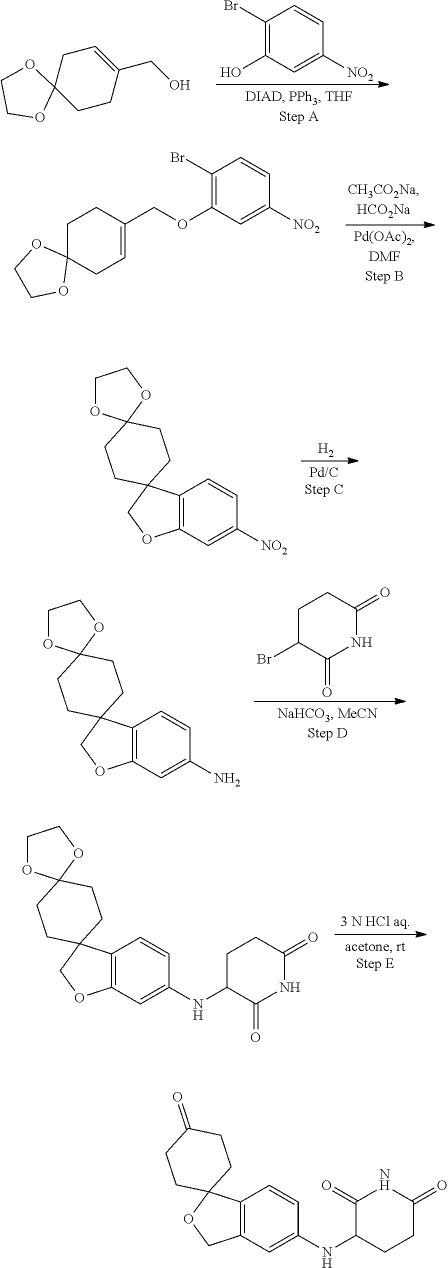

### Step A: 8-((2-bromo-5-nitrophenoxy)methyl)-1,4-dioxaspiro[4.5]dec-7-ene

A mixture of (1,4-dioxaspiro[4.5]dec-7-en-8-yl)methanol (600 mg, 3.53 mmol, 1 eq.; Ref: WO2019074824), 2-bromo-5-nitrophenol (999 mg, 4.58 mmol, 1.3 eq.), triphenylphosphine (1.39 g, 5.29 mmol, 1.5 eq.) and DIAD (0.7 mL, 3.53 mmol, 1 eq.) in THF (5 mL) was stirred at 25° C. for 5 hours. Then the mixture was diluted with water (15 mL) and extracted with EA (15 mL×2). The combined organic layers were washed with brine (20 mL), dried over anhydrous Na₂SO₄ and concentrated under reduced pressure. The residue was purified by flash chromatography and eluted with 0-20% EA in PE to give 8-((2-bromo-5-nitrophenoxy)methyl)-1,4-dioxaspiro[4.5]dec-7-ene (1.1 g, yield 84%) as a white solid. LC-MS (ESI): mass calcd. for C₁₅H₁₆BrNO₅, 369.02; m/z found, 370.0 [M+H]⁺.

### Step B: 6-nitro-2H-dispiro[benzofuran-3,1′-cyclohexane-4′,2″-[1,3]dioxolan]-2′-ene

A mixture of 8-((2-bromo-5-nitrophenoxy)methyl)-1,4-dioxaspiro[4.5]dec-7-ene (300 mg, 810 μmol, 1 eq.), PdOAc₂ (18 mg, 81 mol, 0.1 eq.), sodium formate (61 mg, 891 mol, 1.1 eq.), sodium acetate (166 mg, 2.03 mmol, 2.5 eq.) and TEA (0.15 mL, 891 mol, 1.1 eq.) in DMF (3 mL) was stirred at 95° C. under microwave for 1.5 hours.

Then the mixture was diluted with water (10 mL) and extracted with EA (10 mL×2). The combined organic layers were washed with brine (10 mL), dried over anhydrous Na₂SO₄ and concentrated under reduced pressure. The residue was purified by flash chromatography and eluted with 0-20% EA in PE to give 6-nitro-2H-dispiro[benzofuran-3,1′-cyclohexane-4′,2″-dioxolan]-2′-ene (160 mg, yield 68%) as a yellow solid. LC-MS (ESI): mass calcd. for C₁₅H₁₅NO₅, 289.10; m/z found, 290.2 [M+H]⁺.

### Step C: 2H-dispiro[benzofuran-3,1′-cyclohexane-4′,2″-[1,3]dioxolan]-6-amine

A mixture of 6-nitro-2H-dispiro[benzofuran-3,1′-cyclohexane-4′,2″-[1,3]dioxolan]-2′-ene (50 mg, 173 μmol) and 10% Pd/C (18 mg) in MeOH (5 mL) was stirred at 25° C. under H₂ atmosphere (1 atm) for 4 hours. The mixture solution was filtrated through a Celite pad and the filtrate was concentrated under reduced pressure. The residue was purified by Prep-TLC (PE/EA=1/1) to afford 2H-dispiro[benzofuran-3,1′-cyclohexane-4′,2″-[1,3]dioxolan]-6-amine (35 mg, yield 77%) as a colorless oil. LC-MS (ESI): mass calcd. for C₁₅H₁₉NO₃, 261.14; m/z found, 262.1 [M+H]⁺.

### Step D: 3-((2H-dispiro[benzofuran-3,1′-cyclohexane-4′,2″-[1,3]dioxolan]-6-yl)amino)piperidine-2,6-dione

A mixture of 2H-dispiro[benzofuran-3,1′-cyclohexane-4′,2″-[1,3]dioxolan]-6-amine (100 mg, 383 μmol, 1 eq.), 3-bromopiperidine-2,6-dione (110 mg, 574 μmol, 1.5 eq.) and sodium bicarbonate (96 mg, 1.15 mmol, 3.0 eq.) in MeCN (10 mL) was stirred at 80° C. for 24 hours. The mixture solution was concentrated under reduced pressure to give a residue, then purified by Prep-TLC (PE/EA=1/2) to afford 3-((2H-dispiro[benzofuran-3,1′-cyclohexane-4′,2″-[1,3]dioxolan]-6-yl)amino)piperidine-2,6-dione (80 mg, yield 56%) as a white solid. LC-MS (ESI): mass calcd. for C₂₀H₂₄N₂O₅, 372.17; m/z found, 373.2 [M+H]⁺.

### Step E: 3-((4′-oxo-2H-spiro[benzofuran-3,1′-cyclohexan]-6-yl)amino)piperidine-2,6-dione

A solution of 3-((2H-dispiro[benzofuran-3,1′-cyclohexane-4′,2″-[1,3]dioxolan]-6-yl)amino)piperidine-2,6-dione (60 mg, 161 μmol, 1 eq.) and 3 N HCl aq. in acetone (3 mL) was stirred at 25° C. for 1 hour. The mixture solution was concentrated under reduced pressure to give a residue then purified by Prep-HPLC to afford 3-((4′-oxo-2H-spiro[benzofuran-3,1′-cyclohexan]-6-yl)amino)piperidine-2,6-dione (32 mg, yield 61%) as a white solid. LC-MS (ESI): mass calcd. for C₁₈H₂₀N₂O₄, 328.14; m/z found, 329.1 [M+H]⁺.

¹H NMR (400 MHz, DMSO-d₆) δ 10.76 (s, 1H), 6.96 (d, J=7.9 Hz, 1H), 6.24-6.15 (m, 2H), 5.78 (d, J=7.6 Hz, 1H), 4.49 (s, 2H), 4.30-4.22 (m, 1H), 2.74 (ddd, J=17.5, 12.1, 5.4 Hz, 1H), 2.62-2.51 (m, 2H), 2.47 (s, 1H), 2.26 (d, J=14.8 Hz, 2H), 2.11-1.98 (m, 3H), 1.93-1.78 (m, 3H).

### Compound 18. 3-((5-fluoro-2H-spiro[benzofuran-3,4′-piperidin]-6-yl)amino)piperidine-2,6-dione HCl salt

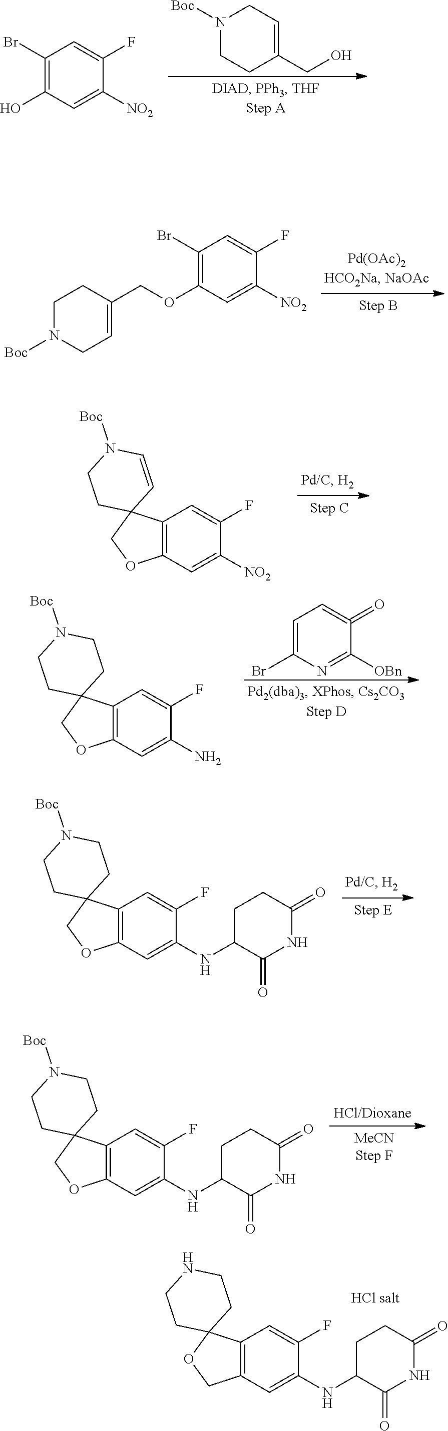

### Step A: tert-butyl 4-((2-bromo-4-fluoro-S-nitrophenoxy)methyl)-3,6-dihydropyridine-1(2H)-carboxylate

To a mixture of 2-bromo-4-fluoro-5-nitrophenol (500 mg, 2.12 mmol, 1 eq.), tert-butyl 4-(hydroxymethyl)-3,6-dihydropyridine-1(2H)-carboxylate (587 mg, 2.75 mmol, 1.3 eq.) and PPh₃ (1.67 g, 6.36 mmol, 3 eq.) in THF (30 mL) was added DIAD (642 mg, 3.18 mmol, 1.5 eq.) at 0° C. Then the reaction mixture was stirred at 50° C. for 16 hours. The mixture was diluted with water (30 mL) and extracted with EA (30 mL×2). The combined organic layers were washed with brine (30 mL), dried over anhydrous Na₂SO₄ and concentrated under reduced pressure. The residue was purified by flash chromatography and eluted with 0-14% EA in PE to give tert-butyl 4-((2-bromo-4-fluoro-5-nitrophenoxy)methyl)-3,6-dihydropyridine-1(2H)-carboxylate (570 mg, yield 62%) as a yellow solid. LC-MS (ESI): mass calcd. for C₁₇H₂₀BrFN₂O₅, 430.05; m/z found, 331.1 [M+H-100]⁺.

### Step B: tert-butyl S-fluoro-6-nitro-2′3′-dihydro-1′H,2H-spiro[benzofuran-3,4′-pyridine]-1′-carboxylate

A mixture of tert-butyl 4-((2-bromo-4-fluoro-5-nitrophenoxy)methyl)-3,6-dihydropyridine-1(2H)-carboxylate (500 mg, 1.16 mmol, 1 eq.), tetraethylammonium chloride (211 mg, 1.28 mmol, 1.1 eq.), sodium acetate (238 mg, 2.9 mmol, 2.5 eq.), sodium formate (86 mg, 1.28 mmol, 1.1 eq.) and palladium diacetate (26 mg, 0.1 eq. 116 mmol) in DMF (10 mL) was stirred at 95° C. under N₂ atmosphere for 4 hours. Then the mixture was diluted with water (50 mL) and extracted with EA (50 mL×2). The combined organic layers were washed with brine (50 mL), dried over anhydrous Na₂SO₄ and concentrated under reduced pressure. The residue was purified by flash chromatography and eluted with 0-20% EA in PE to give tert-butyl 5-fluoro-6-nitro-2′,3′-dihydro-1′H,2H-spiro[benzofuran-3,4′-pyridine]-1′-carboxylate (220 mg, yield 54%) as a yellow solid. LC-MS (ESI): mass calcd. for C₁₇H₁₉FN₂O₅, 350.13; m/z found, 251.1 [M+H-100]⁺.

### Step C: tert-butyl 6-amino-5-fluoro-2H-spiro[benzofuran-3,4′-piperidine]-1′-carboxylate

A mixture of tert-butyl 5-fluoro-6-nitro-2′,3′-dihydro-1′H,2H-spiro[benzofuran-3,4′-pyridine]-1′-carboxylate (220 mg, 628 μmol) and 10% Pd/C (44 mg) in TFE (10 mL) was stirred at 25° C. under H₂ atmosphere (1 atm) for 16 hours. The mixture solution was filtrated through a Celite pad and the filtrate was concentrated under reduced pressure to afford tert-butyl 6-amino-5-fluoro-2H-spiro[benzofuran-3,4′-piperidine]-1′-carboxylate (200 mg, yield 98%) as a white solid. LC-MS (ESI): mass calcd. for C₁₇H₂₃FN₂O₃, 322.17; m/z found, 323.2 [M+H]⁺.

### Step D: tert-butyl 6-((2,6-bis(benzyloxy)pyridin-3-yl)amino)-5-fluoro-2H-spiro[benzofuran-3,4′-piperidine]-1′-carboxylate

A mixture of tert-butyl 6-amino-5-fluoro-2H-spiro[benzofuran-3,4′-piperidine]-1′-carboxylate (160 mg, 496 μmol, 1 eq.), 2,6-bis(benzyloxy)-3-bromopyridine (276 mg, 744 μmol, 1.5 eq.), Pd₂(dba)₃ (45 mg, 49.6 μmol, 0.1 eq.), X-Phos (23 mg, 49.6 μmol, 0.1 eq.) and Cs₂CO₃ (485 mg, 1.49 mmol, 3 eq.) in 1,4-dioxane (15 mL) was stirred at 100° C. under N₂ atmosphere for 16 hours. Then the mixture was diluted with water (50 mL) and extracted with EA (50 mL×2). The combined organic layers were washed with brine (50 mL), dried over anhydrous Na₂SO₄ and concentrated under reduced pressure. The residue was purified by flash chromatography and eluted with 0-25% EA in PE to give tert-butyl 6-((2,6-bis(benzyloxy)pyridin-3-yl)amino)-5-fluoro-2H-spiro[benzofuran-3,4′-piperidine]-1′-carboxylate (190 mg, yield 62%) as a yellow solid. LC-MS (ESI): mass calcd. for C₃₆H₃₈FN₃O₅, 611.28; m/z found, 612.2 [M+H]⁺.

### Step E: tert-butyl 6-((2,6-dioxopiperidin-3-yl)amino)-5-fluoro-2H-spiro[benzofuran-3,4′-piperidine]-1′-carboxylate

A mixture of tert-butyl 6-((2,6-bis(benzyloxy)pyridin-3-yl)amino)-5-fluoro-2H-spiro[benzofuran-3,4′-piperidine]-1′-carboxylate (170 mg, 278 μmol) and 10% Pd/C (33 mg) in TFE (10 mL) was stirred at 25° C. under H₂ atmosphere (1 atm) for 16 hours. The mixture solution was filtrated through a Celite pad and the filtrate was concentrated under reduced pressure. Then the residue was purified flash chromatography and eluted with 0~10% MeOH in DCM to afford tert-butyl 6-((2,6-dioxopiperidin-3-yl)amino)-5-fluoro-2H-spiro[benzofuran-3,4′-piperidine]-1′-carboxylate (100 mg, yield 83%) as a yellow solid. LC-MS (ESI): mass calcd. for C₂₂H₂₈FN₃O₅, 433.20; m/z found, 434.2 [M+H]⁺.

### Step F. 3-((S-fluoro-2H-spiro[benzofuran-3,4′-piperidin]-6-yl)amino)piperidine-2,6-dione HCl salt

A mixture of tert-butyl 6-((2,6-dioxopiperidin-3-yl)amino)-5-fluoro-2H-spiro[benzofuran-3,4′-piperidine]-1′-carboxylate (20 mg, 46.1 gmol) and 4 M HCl/Dioxane (0.3 mL) in MeCN (3 mL) was stirred at 25° C. for 3 hours. The reaction solution was concentrated under reduced pressure to give 3-((5-fluoro-2H-spiro[benzofuran-3,4′-piperidin]-6-yl)amino)piperidine-2,6-dione HCl salt (8.5 mg, yield 50%) as a white solid. LC-MS (ESI): mass calcd. for C₁₇H₂₀FN₃O₃, 333.15; m/z found, 334 [M+H]⁺.

¹H NMR (400 MHz, DMSO-d₆) δ 10.80 (s, 1H), 9.03 (s, 1H), 8.85 (d, J=8.8 Hz, 1H), 6.85 (d, J=11.0 Hz, 1H), 6.35 (d, J=7.1 Hz, 1H), 4.39 (s, 2H), 4.38-4.35 (m, 1H), 3.29-3.25 (m, 2H), 3.00-2.91 (m, 2H), 2.80-2.71 (m, 1H), 2.58-2.53 (m, 1H), 2.03-1.96 (m, 3H), 1.80-1.74 (m, 3H).

### Compound 19. 3-((4-fluoro-2H-spiro[benzofuran-3,4′-piperidin]-6-yl)amino)piperidine-2,6-dione HCl salt

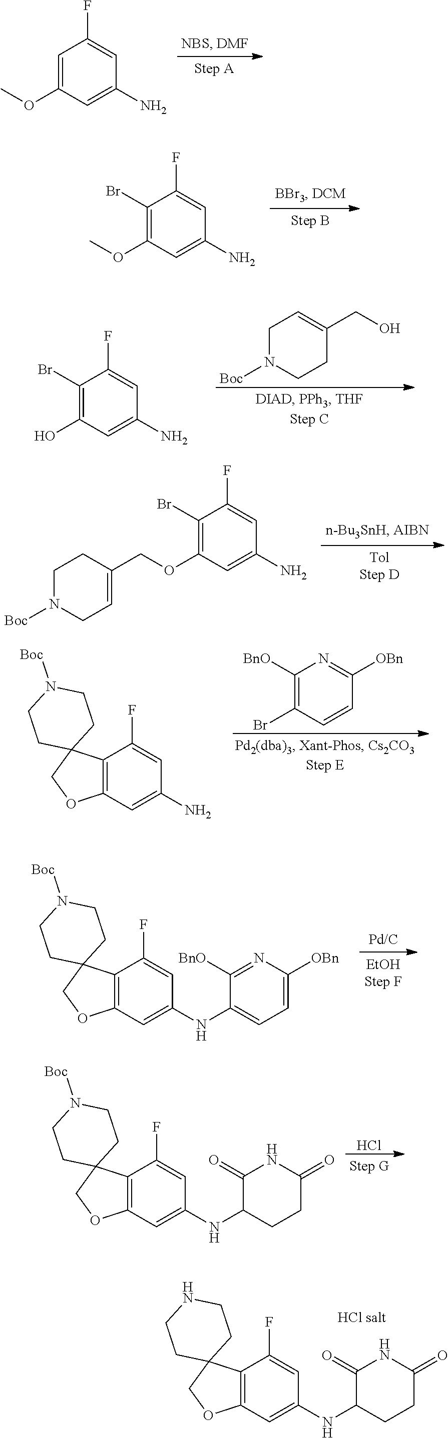

### Step A: 4-bromo-3-fluoro-S-methoxyaniline

A mixture of 3-fluoro-5-methoxyaniline (8.9 g, 63.1 mmol, 1 eq.) and NBS (11.2 g, 63.1 mmol, 1 eq.) in DMF (400 mL) was stirred at 25° C. for 1 hour. Then the mixture was poured into water (2 L) and the precipitate was filtered to afford 4-bromo-3-fluoro-5-methoxyaniline (12.5 g, yield 90%) as a yellow solid. LC-MS (ESI): mass calcd. for C₇H₇BrFNO, 218.97; m/z found, 220.1/222.1 [M+H]⁺.

¹H NMR (400 MHz, DMSO-d₆) δ 6.15 (s, 1H), 6.11 (dd, J=11.2, 1.8 Hz, 1H), 5.61 (s, 2H), 3.76 (s, 3H).

### Step B: 5-amino-2-bromo-3-fluorophenol

To a solution of 4-bromo-3-fluoro-5-methoxyaniline (3.2 g, 14.5 mmol, 1 eq.) in DCM (30 mL) was added BBr₃ (6.9 mL, 72.7 mmol, 5 eq.) at 0° C. The reaction mixture was stirred at room temperature for 1 hour. After quenched with water (10 mL) at 0° C., the mixture was diluted with water (30 mL) and extracted with DCM (30 mL×2). The combined organic layers were washed with sat. NaHCO₃ aq. (30 mL) and brine (30 mL), dried over anhydrous Na₂SO₄ and concentrated under reduced pressure. The residue was purified by flash chromatography and eluted with 0-30% EA in PE to give 5-amino-2-bromo-3-fluorophenol (2.8 g, yield 94%) as a yellow solid. LC-MS (ESI): mass calcd. for C₆H₅BrFNO, 204.95; m/z found, 206.1 [M+H]⁺.

### Step C: tert-butyl 4-((5-amino-2-bromo-3-fluorophenoxy)methyl)-3,6-dihydropyridine-1(2H)-carboxylate

To a solution of 5-amino-2-bromo-3-fluorophenol (483 mg, 2.34 mmol, 1 eq.), tert-butyl 4-(hydroxymethyl)-3,6-dihydropyridine-1(2H)-carboxylate (500 mg, 2.34 mmol, 1 eq.) and triphenylphosphine (922 mg, 3.52 mmol, 1.5 eq.) in THF (20 mL) was added DIAD (0.7 mL, 3.52 mmol, 1.5 eq.) at 0° C. Then the reaction mixture was stirred at room temperature for 18 hours. Then the mixture was diluted with water (50 mL) and extracted with EA (50 mL×2). The combined organic layers were washed with brine (50 mL), dried over anhydrous Na₂SO₄ and concentrated under reduced pressure. The residue was purified by flash chromatography and eluted with 0-30% EA in PE to give tert-butyl 4-((5-amino-2-bromo-3-fluorophenoxy)methyl)-3,6-dihydropyridine-1(2H)-carboxylate (900 mg, yield 96%) as a yellow solid. LC-MS (ESI): mass calcd. for C₁₇H₂₂BrFN₂O₃, 400.08; m/z found, 401.1 [M+H]⁺.

### Step D: tert-butyl 6-amino-4-fluoro-2H-spiro[benzofuran-3,4′-piperidine]-1′-carboxylate

A mixture of tert-butyl 4-((5-amino-2-bromo-3-fluorophenoxy)methyl)-3,6-dihydropyridine-1(2H)-carboxylate (800 mg, 1.99 mmol, 1 eq.), 2,2′-Azobisisobutyronitrile (32.7 mg, 199 μmol, 0.1 eq.) and Tributyltin hydride (3.24 mL, 12.0 mmol, 6 eq.) in toluene (20 mL) was stirred at 110° C. for 18 hour. The reaction was quenched with sat. KF aq. (10 mL) and diluted with water (50 mL) and extracted with EA (50 mL×2). The combined organic layers were washed with brine (50 mL), dried over anhydrous Na₂SO₄ and concentrated under reduced pressure. The residue was purified by flash chromatography and eluted with 0-20% EA in PE to give tert-butyl 6-amino-4-fluoro-2H-spiro[benzofuran-3,4′-piperidine]-1′-carboxylate (181 mg, yield 28%) as a yellow solid. LC-MS (ESI): mass calcd. for C₁₇H₂₃FN₂O₃, 322.17; m/z found, 322.2 [M+H]⁺.

### Step E: tert-butyl 6-((2,6-bis(benzyloxy)pyridin-3-yl)amino)-4-fluoro-2H-spiro[benzofuran-3,4′-piperidine]-1′-carboxylate

A mixture of tert-butyl 6-amino-4-fluoro-2H-spiro[benzofuran-3,4′-piperidine]-1′-carboxylate (90 mg, 279 μmol, 1 eq.), 2,6-bis(benzyloxy)-3-bromopyridine (124 mg, 335 μmol, 1.2 eq.), Pd₂(dba)₃ (26 mg, 27.9 μmol, 0.1 eq.), 2-(dicyclohexylphosphanyl)-2′,4′,6′-tris(isopropyl)biphenyl (27 mg, 55.8 μmol, 0.2 eq.) and Cs₂CO₃ (273 mg, 838 μmol, 3 eq.) in 1,4-dioxane (5 mL) was stirred at 100° C. for 18 hours. Then the mixture was diluted with water (20 mL) and extracted with EA (20 mL×2). The combined organic layers were washed with brine (20 mL), dried over anhydrous Na₂SO₄ and concentrated under reduced pressure. The residue was purified by flash chromatography and eluted with 0-15% EA in PE to give tert-butyl 6-((2,6-bis(benzyloxy)pyridin-3-yl)amino)-4-fluoro-2H-spiro[benzofuran-3,4′-piperidine]-1′-carboxylate (128 mg, yield 75%) as a yellow solid. LC-MS (ESI): mass calcd. for C₃₆H₃₈FN₃O₅, 611.28; m/z found, 611.3 [M+H]⁺.

### Step F: tert-butyl 6-((2,6-dioxopiperidin-3-yl)amino)-4-fluoro-2H-spiro[benzofuran-3,4′-piperidine]-1′-carboxylate

A mixture of tert-butyl 6-((2,6-bis(benzyloxy)pyridin-3-yl)amino)-4-fluoro-2H-spiro[benzofuran-3,4′-piperidine]-1′-carboxylate (100 mg, 163 μmol) and 10% Pd—C(150 mg) in EtOH (6 mL) was stirred at 45° C. under H₂ atmosphere (1 atm) for 3 hours. The mixture solution was filtrated through a Celite pad and the filtrate was concentrated under reduced pressure to afford tert-butyl 6-((2,6-dioxopiperidin-3-yl)amino)-4-fluoro-2H-spiro[benzofuran-3,4′-piperidine]-1′-carboxylate (43 mg, yield 61%) as a blue solid. LC-MS (ESI): mass calcd. for C₂₂H₂₈FN₃O₅, 433.2; m/z found, 434.2 [M+H]⁺.

### Step G: 3-((4-fluoro-2H-spiro[benzofuran-3,4′-piperidin]-6-yl)amino)piperidine-2,6-dione HCl salt

A mixture of tert-butyl 6-((2,6-dioxopiperidin-3-yl)amino)-4-fluoro-2H-spiro[benzofuran-3,4′-piperidine]-1′-carboxylate (43 mg, 99.2 μmol) and 4 M HCl in dioxane (1 mL) in DCM (5 mL) was stirred at 25° C. for 0.5 hour. The mixture solution was concentrated under reduced pressure to give a residue then purified by Prep-HPLC to give 3-((4-fluoro-2H-spiro[benzofuran-3,4′-piperidin]-6-yl)amino)piperidine-2,6-dione HCl salt (12.5 mg, yield 34%) as a blue solid. LC-MS (ESI): mass calcd. for C₁₇H₂₀FN₃O₃, 333.15; m/z found, 334.1 [M+H]⁺.

¹H NMR (400 MHz, DMSO-d₆) δ 10.79 (s, 1H), 9.19 (d, J=9.6 Hz, 1H), 8.75 (d, J=7.6 Hz, 1H), 6.04-5.97 (m, 2H), 4.44 (s, 2H), 4.33-4.27 (m, 1H), 3.29-3.23 (m, 2H), 2.97-2.86 (m, 2H), 2.78-2.68 (m, 1H), 2.60-2.52 (m, 1H), 2.25-2.17 (m, 2H), 2.09-2.01 (m, 1H), 1.88-1.78 (m, 3H).

### II. Biological Assessment

### In Vitro Assay: IC₅₀ Measurements for Binding to CRBN/DDB1

The binding potency was determined using HTRF assay technology (Perkin Elmer). Compounds were serially diluted in DMSO and 0.2 μL volume was transferred to white 384-well plate. The reaction was conducted in total volume of 20 μL with addition of 2 nM His tagged CRBN+DDB-DLS7+CXU4 (Wuxi, catalogue #RP210521GA) to compounds followed by addition of 60 nM Fluorescent probe Cy5-labeled Thalidomide (Tenova Pharma, catalogue #T52461), and 0.4 nM of MAb Anti-6HIS Tb cryptate Gold (Cisbio, catalogue #61HI2TLA in the assay buffer (50 mM HEPES pH 7.5, 1 mM TCEP, 0.01% Brij-35, 50 mM NaCl, and 0.1% BSA). After one hour incubation at room temperature, the HTRF signals were read on Envision reader (Perkin Elemer). Data was analyzed using XLfit using four parameters dose response curve to determine IC₅₀s.

TABLE E1
Binding IC₅₀ to CRBN/DDB1

| Compound No. | CRBN HTRF IC₅₀ (nM) |
|---|---|
| 1 | C |
| 2 | C |
| 3 | C |
| 4 | C |
| 5 | C |
| 6 | C |
| 7 | D |
| 8 | C |
| 9 | C |
| 10 | D |
| 11 | B |
| 12 | C |
| 13 | D |
| 14 | C |
| 15 | C |
| 16 | C |
| 17 | C |
| 18 | C |
| 19 | B |

Note:
IC₅₀: “A”: <50 nM; “B”: 50-500 nM; “C”: >500 and <5000 nM; “D”: >=5000 nM.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula II: 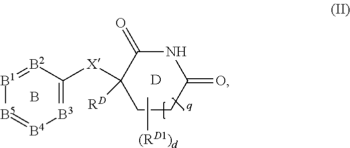
or a pharmaceutically acceptable salt, solvate, or stereoisomer thereof, wherein:
B¹ is N or CR^{B1};
B² is N or CR^{B2};
B³ is N or CR^{B3};
B⁴ is N or CR^{B4};
B⁵ is N or CR^{B5};
wherein:
i) B⁵ is CR^{B5}, and either R^{B1} and R^{B5} or R^{B4} and R^{B5}, together with the carbon atoms to which they are bonded, form Ring A, wherein Ring A is optionally substituted C₅₋₁₆ fused or spiro carbocycle or 5- to 16-membered fused or spiro heterocycle;
the remaining of R^{B1}, R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
or
ii) R^{B5} is 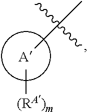
wherein:
Ring A′ is C₄₋₁₂ fused carbocyclyl or 4- to 12-membered fused heterocyclyl;
each R^{A′} is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 10, as valency permits;
the remaining of R^{B1}, R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
X′ is —O— or —NR^{X′}—;
R^{X′} is hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u};
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 6;
R^{D} is hydrogen, deuterium, C₁₋₆ haloalkyl, or C₁₋₆ alkyl; and
q is an integer selected from 0 to 2,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

2. A conjugate of Formula II: 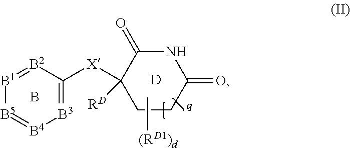
or a pharmaceutically acceptable salt, solvate, or stereoisomer thereof,
wherein:
B¹ is N or CR^{B1};
B² is N or CR^{B2};
B³ is N or CR^{B3};
B⁴ is N or CR^{B4};
B⁵ is N or CR^{B5};
wherein:
i) B⁵ is CR^{B5}, and either R^{B1} and R^{B5} or R^{B4} and R^{B5}, together with the carbon atoms to which they are bonded, form Ring A attached to -L-T, wherein Ring A is optionally substituted C₅₋₁₆ fused or spiro carbocycle or 5- to 16-membered fused or spiro heterocycle;
the remaining of R^{B1}, R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
or
ii) R^{B5} is 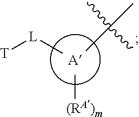
wherein:
Ring A is C₄₋₁₂ fused carbocyclyl or 4- to 12-membered fused heterocyclyl;
each R^{A} is independently amino-protecting group, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 10, as valency permits;
the remaining of R^{B1}, R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
X′ is —O— or —NR^{X′}—;
R^{X′} is hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u};
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 6;
R^{D} is hydrogen, deuterium, C₁₋₆ haloalkyl, or C₁₋₆ alkyl; and
q is an integer selected from 0 to 2,
L is a linker; and
T is a ligand for a protein,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

3. The compound or conjugate of claim 1 or 2, wherein B⁵ is CR^{B5}, and either R^{B1} and R^{B5} or R^{B4} and R^{B5}, together with the carbon atoms to which they are bonded, form Ring A or Ring A attached to -L-T.

4. The compound or conjugate of any one of claims 1-3, wherein
Ring A is: 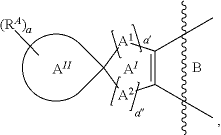
Ring A attached to -L-T is 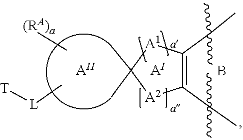
wherein:
Ring A^{II} is C₃₋₈ carbocycle or 3- to 8-membered heterocycle;
each A¹ is independently —C(R^{A1})₂—, —NR^{A1′}—, —O—, —S—, —S(═O)—, or —S(═O)₂—;
each A² is independently —C(R^{A2})₂—, —NR^{A2′}—, —O—, —S—, —S(═O)—, or —S(═O)₂—;
each occurrence of R^{A1} and R^{A2} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{A1} or two geminal R^{A2} together form an oxo; or
two geminal R^{A1} or two geminal R^{A2}, together with the carbon atom to which they are attached, form C₃₋₆ carbocycle or 3- to 6-membered heterocycle, wherein the carbocycle or heterocycle is optionally substituted with one or more R^{u};
each occurrence of R^{A1′} and R^{A2′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
a′ and a″ are independently an integer selected from 0 to 2, wherein a′ and a″ are not both 0;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
a is an integer selected from 0 to 8, as valency permits.

5. The compound or conjugate of claim 4, wherein Ring A^{I} is not carbocycle.

6. The compound or conjugate of claim 4, wherein Ring A^{I} is 5- to 8-membered heterocycle.

7. The compound or conjugate of any one of claims 4-6, wherein each A¹ is independently —C(R^{A1})₂—, —NR^{A1′}—, or —O—, and each A² is independently —C(R^{A2})₂—, —NR^{A2′}—, or —O—.

8. The compound or conjugate of any one of claims 4-7, wherein
each occurrence of R^{A1} and R^{A2} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; and
each occurrence of R^{A1′} and R^{A2′} is independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

9. The compound or conjugate of any one of claims 4-8, wherein each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂-6 alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

10. The compound or conjugate of claim 9, wherein a is 0 or 1.

11. The compound or conjugate of any one of claims 4-10, wherein
Ring A is: 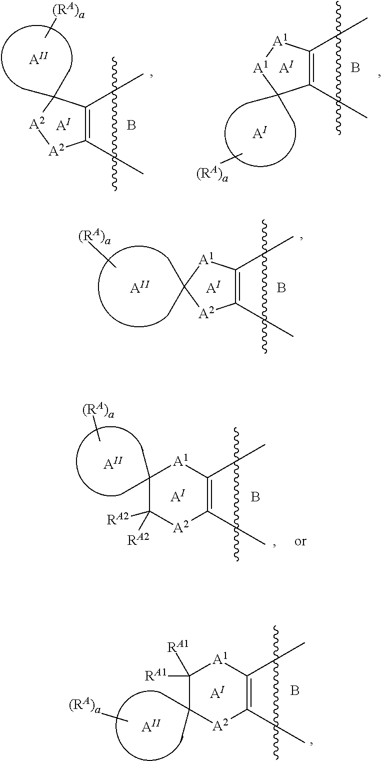
or
Ring A attached to -L-T is

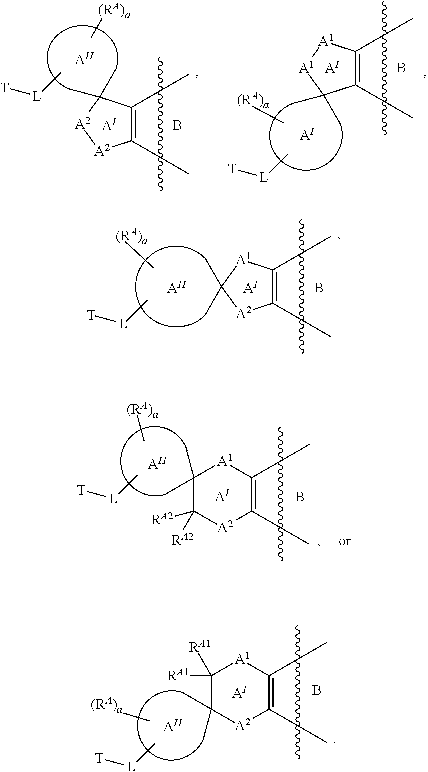

12. The compound or conjugate of claim 11, wherein
the compound of Formula II is a compound of Formula II-1 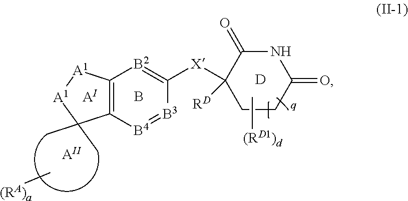
or
the conjugate of Formula II is a conjugate of Formula II-2

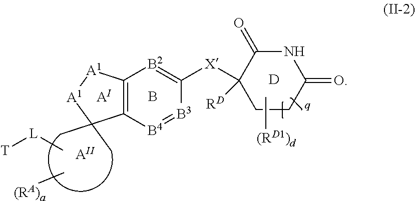

13. The compound or conjugate of any one of claims 1-3, wherein
Ring A is 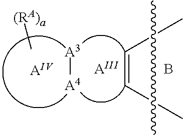
or
Ring A attached to -L-T is 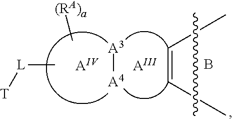
wherein:
Ring A^{III} and Ring A^{IV} are independently C₄₋₈ carbocycle or 4- to 8-membered heterocycle;
A³ and A⁴ are independently C, CRAX, or N;
R^{Ax} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
a is an integer selected from 0 to 8, as valency permits,
wherein each R^{A} may be present on either Ring A^{III} or Ring A^{IV}.

14. The compound or conjugate of claim 13, wherein
Ring A is 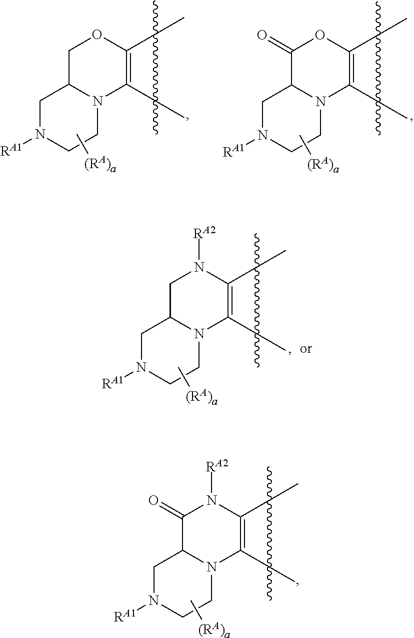
Ring A attached to L-T is 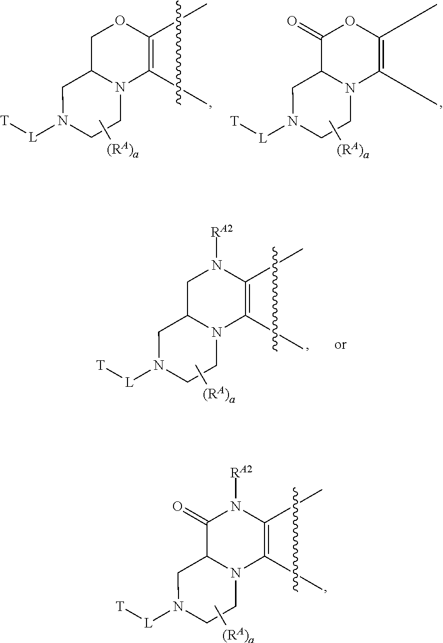
wherein:
R^{A5} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R^{A5} is an amino-protecting group; and
R^{A6} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; or
R^{A6} is an amino-protecting group.

15. The compound or conjugate of claim 14, wherein R^{A5} is hydrogen, C₁₋₆ alkyl, —C(═O)R^{a}, or —C(═O)OR^{b}, or R^{A5} is an amino-protecting group.

16. The compound or conjugate of claim 14 or 15, wherein R^{A6} is hydrogen or C₁₋₆ alkyl.

17. The compound or conjugate of claim 14, wherein
the compound of Formula II is a compound of Formula II-3 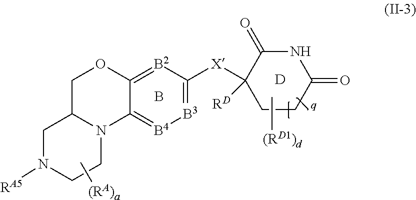
the conjugate of Formula II is a conjugate of Formula II-4

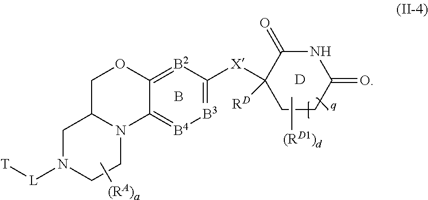

18. The compound or conjugate of any one of claims 13-17, wherein each R^{A} i independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

19. The compound or conjugate of claim 18, wherein a is 0 or 1.

20. The compound or conjugate of claim 1 or 2, wherein
R^{B5} is 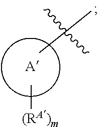
or

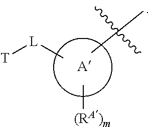

21. The compound or conjugate of claim 20, wherein
the compound of Formula II is a compound of Formula II-5 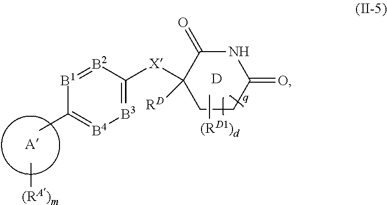
the conjugate of Formula II is a conjugate of Formula II-6

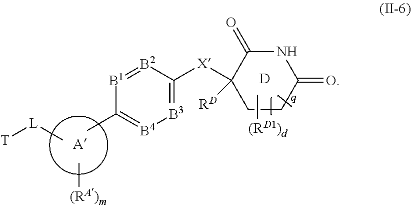

22. The compound or conjugate of claim 20 or 21, wherein Ring A′ is 4- to 12-membered fused heterocyclyl comprising 1 or 2 nitrogen atoms.

23. The compound or conjugate of any one of claims 20-22, wherein each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

24. The compound or conjugate of any one of claims 20-23, wherein at least one R^{A′} is halogen.

25. The compound or conjugate of any one of claims 20-24, wherein m is an integer selected from 0 to 3.

26. The compound or conjugate of claim 20, wherein
R^{B5} is 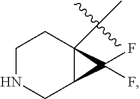 [SMILES: *[C@@]12CCNC[C@@H]1C2(F)F]
or

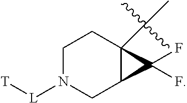

21. The compound or conjugate of any one of claims 1-20, wherein the remaining of R^{B1}, R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

22. The compound or conjugate of any one of claims 1-20, wherein the remaining of R^{B1}, R^{B2}, R^{B3}, R^{B4}, and R^{B5}, when applicable, are independently hydrogen, halogen, or C₁₋₆ alkyl.

25. The compound or conjugate of any one of claims 1-24, wherein X′ is O.

26. The compound or conjugate of any one of claims 1-24, wherein X′ is —NR^{X′}—, wherein R^{X′} is hydrogen.

27. The compound or conjugate of any one of claims 1-26, wherein d is 0.

28. The compound or conjugate of any one of claims 1-27, wherein R^{D} is hydrogen.

29. The compound or conjugate of any one of claims 1-28, wherein q is 1.

30. A compound selected from the compounds in Table 1 or a pharmaceutically acceptable salt thereof.

31. A pharmaceutical composition comprising the compound of any one of claims 1 and 3-30, and a pharmaceutically acceptable excipient.

32. A method of binding cereblon E3 ubiquitin ligase protein complex in a subject or biological sample comprising administering the compound of any one of claims 1 and 3-30 to the subject or contacting the biological sample with the compound of any one of claims 1 and 3-30.

33. Use of the compound of any one of claims 1 and 3-30 in the manufacture of a medicament for binding cereblon E3 ubiquitin ligase protein complex in a subject or biological sample.

34. A compound of any one of claims 1 and 3-30 for use in binding cereblon E3 ubiquitin ligase protein complex in a subject or biological sample.

35. A method of degrading a protein in a subject or biological sample comprising administering the conjugate of any one of claims 2-29 to the subject or contacting the biological sample with the conjugate of any one of claims 2-29.

36. Use of the conjugate of any one of claims 2-29 in the manufacture of a medicament for degrading a protein in a subject or biological sample.

37. A conjugate of any one of claims 2-29 for use in degrading a protein in a subject or biological sample.

38. The method, use, or conjugate for use of any one of claims 35-37, wherein the protein is an estrogen receptor, a STAT3 protein, an androgen receptor, a SMARCA2 protein, a SMARCA4 protein, a BRD9 protein, or a CBP/p300 protein.
